(19) United States
(12) Patent Application Publication
(10) Pub. No.: US 2015/0094354 A1
(43) Pub. Date: Apr. 2, 2015

(54) METHOD FOR TREATING CELL PROLIFERATIVE DISORDER BY INHIBITING C1GALT1 EXPRESSION

(71) Applicant: National Taiwan University, Taipei City (TW)

(72) Inventors: Min-Chuan HUANG, Taipei City (TW); Yao-Ming WU, Taipei City (TW); Chiung-Hui LIU, Taipei City (TW); Chih-Hsing CHOU, Taipei City (TW); Miao-Juei HUANG, Taipei City (TW); John HUANG, Taipei City (TW); Ji-Shiang HUNG, Taipei City (TW); Chiun-Sheng HUANG, Taipei City (TW); Hsueh-Fen JUAN, Taipei City (TW)

(21) Appl. No.: 14/500,152

(22) Filed: Sep. 29, 2014

Related U.S. Application Data
(60) Provisional application No. 61/884,398, filed on Sep. 30, 2013.

(51) Int. Cl. A61K 31/496; G01N 33/50; C12N 15/113

(52) U.S. Cl. CPC A61K 31/496; C12N 15/1137; G01N 33/5011; C12N 2310/14; G01N 2333/91102; G01N 2500/10; G01N 2500/04
USPC 514/44 A; 514/254.07; 435/15; 435/6.13

(57) ABSTRACT

A method for treating a cell proliferative disorder in a subject is provided. The method for treating a cell proliferative disorder has a step of: administering a C1GALT1 inhibition substance to the subject for inhibiting C1GALT1 expression or activity in the subject, so as to treat the cell proliferative disorder in the subject.

### CROSS REFERENCE TO RELATED APPLICATIONS

This application claims the benefit of priority under 35 USC 119(e) of U.S. Provisional Application No. 61/884,398, filed on Sep. 30, 2013, the contents of which are incorporated herein by reference in their entirety.

This invention is partly disclosed in a thesis entitled “C1GALT1 Promotes Invasive Phenotypes of Hepatocellular Carcinoma Cells by Modulating Integrin β1 Glycosylation and Activity” on Aug. 4, 2014.

This invention is partly disclosed in a thesis entitled “C1GALT1 overexpression promotes the invasive behavior of colon cancer cells through modifying O-glycosylation of FGFR2” on Mar. 15, 2014.

This invention is partly disclosed in a thesis entitled “C1GALT1 Enhances Proliferation of Hepatocellular Carcinoma Cells via Modulating MET Glycosylation and Dimerization” on Jul. 5, 2013.

This invention is partly disclosed in a conference poster entitled “Up-regulation of core 1 β1,3-galactosyltransferase enhances malignant phenotypes of hepatocellular carcinoma cells by activating the c-MET pathway” on Oct. 1, 2012.

This invention is partly disclosed in a conference poster entitled “Up-regulation of C1GALT1 promotes breast cancer cell growth through MUC1-C signaling pathway,” on Jul. 5, 2014.

This invention is partly disclosed in a conference poster entitled “T synthase Glycosylates and Activates c-MET in Hepatocellular Carcinoma Cells” on Jun. 23, 2013.

### FIELD OF THE INVENTION

The present invention relates to a method for treating a cell proliferative disorder, in particular by inhibiting C1GALT1 expression or activity.

### BACKGROUND OF THE INVENTION

Glycosylation is the most common posttranslational modification of proteins. There are 2 types of glycosylation, N-linked and O-linked. Aberrant glycosylation is often observed in cancers). Accumulated evidence indicates that alterations in N-linked glycosylation are a hallmark of various liver diseases, including hepatocellular carcinoma. For instance, the expression of N-acetylglucosaminyltransferase-III and -V is increased in hepatocellular carcinoma. An N-glycan profiling study identified novel N-glycan structures in serum as prognostic markers of hepatocellular carcinoma. In addition, a-1,6-fucosyltransferase can generate fucosylated a-fetoprotein (AFP), which provided a more accurate diagnosis of hepatocellular carcinoma from chronic liver diseases. However, changes in O-linked glycosylation have been overlooked in the past. The O-glycosylation of proteins is difficult to explore, as consensus amino acid sequences of O-glycosylation remain unclear and effective releasing enzymes for O-glycans are not available. Mucin-type O-glycosylation is the most common O-linked glycosylation. Recently, a systematic analysis of mucin-type O-linked glycosylation revealed that mucin type O-glycans are decorated not only on mucins but on various unexpected proteins. Functions of the O-glycosylation are largely unknown. Several lines of evidence indicate that O-glycosylation of proteins plays critical roles in cancer. O-glycans on major histocompatibility complex class I-related chain A (MICA) enhance bladder tumor metastasis, and O-glycosylation of death receptor controls apoptotic signaling in several types of cancer. During O-glycosylation, the N-Acetylgalactosaminyltransferase (GALNTs) family enzymes initiate the addition of N-Acetylgalactosamine (GalNAc) to (Ser) or threonine (Thr) residues to form GalNAc-O-Ser/Thr (or GalNAc-Ser/Thr) structure, also known as Tn antigen. Subsequently, Core 1 β1,3-galactosyltransferase (C1GALT1), which is a critical mucin-type O-glycosyltransferase and localized in the Golgi apparatus, transfers galactose (Gal) to GalNAc to a serine (Ser) or threonine (Thr) to form Gal-GalNAc-O-Ser/Thr (Gal-GalNAc-Ser/Thr) structure, known as T antigen or core 1 structure. The core 1 structure (T antigen) is the precursor for subsequent glycosidic branching, extension and maturation of mucin-type O-glycans. Our previous study showed that GALNT2 regulates EGF (epidermal growth factor) receptor activity and cancer in hepatocellular carcinoma cells. Therefore, understanding the roles of O-glycosylation in cancer may provide novel insights into the pathogenesis of cancer. On the other hand, C1GALT1 has been shown to regulate angiogenesis, thrombopoiesis, and kidney development. Although mucin-type O-glycosylation and C1GALT1 have been shown to play crucial roles in a variety of biologic functions, the expression and the role of C1GALT1 in cancer remain unclear.

### SUMMARY OF THE INVENTION

A primary object of the present invention is to provide a method for treating a cell proliferative disorder by inhibiting C1GALT1 expression or activity. The cell proliferative disorder includes a tumor or a cancer.

To achieve the above object, the present invention provides a method for treating a cell proliferative disorder in a subject, comprising a step of: administering a C1GALT1 inhibition substance to the subject for inhibiting C1GALT1 expression or activity in the subject, so as to treat the cell proliferative disorder in the subject.

In an embodiment of the present invention, the C1GALT1 inhibition substance further inhibits phosphorylation or dimerization of RTKs (receptor tyrosine kinases).

In an embodiment of the present invention, the C1GALT1 inhibition substance further alters glycosylation of RTKs (receptor tyrosine kinases).

Furthermore, the RTKs are selected from a group consisting of mesenchymal epithelial transition factor (MET, also known as hepatocyte growth factor receptor, HGFR), and FGFRs (fibroblast growth factor receptor).

In an embodiment of the present invention, the C1GALT1 inhibition substance further alters glycosylation of MUC1 (mucin 1).

In an embodiment of the present invention, the C1GALT1 inhibition substance comprises an antisense nucleotide sequence complementary to all or a part of C1GALT1 mRNA.

Furthermore, the antisense nucleotide sequence comprises:

(SEQ ID NO: 1)
UUAGUAUACGUUCAGGUAAGGUAGG;
or
(SEQ ID NO: 2)
UUAUGUUGGCUAGAAUCUGCAUUGA.

Furthermore, a concentration of the antisense nucleotide sequence administered to the subject is ranged from 0.05 nM to 1000 nM.

In an embodiment of the present invention, the cell proliferative disorder is selected from the group consisting of hepatocellular carcinoma, colorectal cancer, breast cancer, head and neck squamous cell carcinoma, lung cancer, ovarian cancer, endometrial cancer, and cholangiocarcinoma.

In an embodiment of the present invention, the cell proliferative disorder is selected from the group consisting of cell migration, cell invasion, and tumor metastasis.

In another preferable embodiment of the present invention, the C1GALT1 inhibition substance is a small molecule substance, and the molecular weight thereof is less than 900 Daltons.

In an embodiment of the present invention, the C1GALT1 inhibition substance binds to a catalytic domain of C1GALT1.

In an embodiment of the present invention, the C1GALT1 inhibition substance leads to Tn antigen accumulation in cancer cells.

In an embodiment of the present invention, the C1GALT1 inhibition substance decreases T antigen formation in cancer cells.

In an embodiment of the present invention, the C1GALT1 inhibition substance is itraconazole.

In an embodiment of the present invention, the small molecular substance administered to the subject is ranged from 1 ug/ml to 100 ug/ml.

Another object of the present invention is to provide a method for treating a cell proliferative disorder by administering a C1GALT1 inhibition substance in combination with an anti-tumor drug for producing a synergetic effect.

To achieve the above object, before or after administering the C1GALT1 inhibition substance to the subject, the method for treating a cell proliferative disorder in a subject further comprises a step of: administering an anti-tumor drug to the subject for producing a synergetic effect.

Another object of the present invention is to provide a method for identifying a potential compound for therapeutically treating a cell proliferative disorder.

To achieve the above object, the present invention also provides a method for identifying a potential compound for therapeutically treating a cell proliferative disorder, comprising steps of:

administering a to-be-tested compound to cells having the cell proliferative disorder;

examining the activity and expression of C1GALT1 in the cells; and

determining whether the to-be-tested compound is the potential compound for therapeutically treating the cell proliferative disorder, wherein when the activity or expression of C1GALT1 in the cells after being treated with the to-be-tested compound is lower than a predetermined percentage of the activity and expression of the cells before being treated with the to-be-tested compound, the to-be-tested compound is determined to be the potential compound for the treating cell proliferative disorder.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a schematic diagram illustrating mRNA expression of C1GALT1 in paired hepatocyte carcinoma tissues and non-tumor liver tissues analyzed by quantitative real-time RT-PCR (Q-PCR) in accordance with an embodiment of the present invention.

FIG. 2 is an autoradiogram illustrating C1GALT1 expression in paired hepatocyte carcinoma tissues and non-tumor liver tissues analyzed by Western blotting in accordance with an embodiment of the present invention.

FIG. 3A is an image illustrating C1GALT1 expression in hepatocellular carcinoma tissues and non-tumor liver tissues analyzed by immunohistochemistry in accordance with an embodiment of the present invention.

FIG. 3B-3C are amplified images of regions of FIG. 3A.

FIG. 4A is a liver tissue image illustrating different intensity of C1GALT1 staining in accordance with an embodiment of the present invention.

FIG. 4B is a schematic diagram illustrating percentages of cases with different intensity levels of C1GALT1 immunohistochemistry staining in hepatocellular carcinoma and hepatocytes in accordance with an embodiment of the present invention.

FIG. 5 is a schematic diagram illustrating overall survival probabilities for patients with hepatocellular carcinoma with high and low C1GALT1 expression in accordance with an embodiment of the present invention.

FIG. 6A is an autoradiogram illustrating C1GALT1 expression in seven hepatocyte carcinoma cell lines and nine non-tumor liver tissues analyzed by Western blotting in accordance with an embodiment of the present invention.

FIG. 6B is a schematic diagram illustrating the quantified C1GALT1 expression signals from FIG. 6A in accordance with an embodiment of the present invention.

FIG. 7A is an autoradiogram illustrating C1GALT1 expression and the effects of C1GALT1 on O-glycosylation of glycoproteins in hepatocellular carcinoma cell lines of a control group and C1GALT1 knockdown groups in accordance with an embodiment of the present invention.

FIG. 7B is an autoradiogram illustrating C1GALT1 expression and the effects of C1GALT1 on O-glycosylation of glycoproteins in hepatocellular carcinoma cell lines of a control group and a C1GALT1 overexpression group in accordance with an embodiment of the present invention.

FIG. 8 is a graph illustrating surface O-glycans of hepatocellular carcinoma cell surfaces analyzed by flow cytometry with FITC-VVA in accordance with an embodiment of the present invention.

FIGS. 9A-9B are schematic diagrams illustrating the effects of C1GALT1 on cell viability of hepatocellular carcinoma cells in vitro in accordance with an embodiment of the present invention.

FIGS. 10A-10B are schematic diagrams illustrating the effects of C1GALT1 on cell proliferation of hepatocellular carcinoma cells in vitro in accordance with an embodiment of the present invention.

FIGS. 11A-11C are schematic diagrams and images illustrating the effects of C1GALT1 on tumor growth of hepatocellular carcinoma in SCID mouse model in vivo in accordance with an embodiment of the present invention.

FIG. 12 is an autoradiogram illustrating the effects of C1GALT1 on the phosphorylation of RTKs in hepatocellular carcinoma cells in accordance with an embodiment of the present invention.

FIGS. 13A-13B are autoradiograms illustrating the effects of C1GALT1 on the HGF-induced signaling and IGF-induced signaling in hepatocellular carcinoma cells in accordance with an embodiment of the present invention.

FIG. 14 is schematic diagrams illustrating the effects of the MET inhibitor, PHA665752, on C1GALT1-enhanced cell viability of hepatocellular carcinoma cells in accordance with an embodiment of the present invention.

FIG. 15 is an autoradiogram comparing C1GALT1 expression with MET phosphorylation in hepatocellular carcinoma tissues in accordance with an embodiment of the present invention.

FIG. 16 is a graph illustrating the correlation of C1GALT1 expression and MET phosphorylation in 20 hepatocellular carcinoma tumors in accordance with an embodiment of the present invention.

FIG. 17 is an autoradiogram illustrating the N-glycan and the sialyl Tn antigen decoration on MET in accordance with an embodiment of the present invention.

FIGS. 18A-18B are autoradiograms illustrating the effects of C1GALT1 on O-glycosylation on MET in hepatocellular carcinoma cells in accordance with an embodiment of the present invention.

FIGS. 19A-19B are autoradiograms illustrating the effects of C1GALT1 on dimerization of MET in hepatocellular carcinoma cells in accordance with an embodiment of the present invention.

FIG. 20 is an autoradiogram illustrating C1GALT1 expression in colorectal cancer tissues and non-tumor tissues analyzed by Western blotting in accordance with an embodiment of the present invention.

FIG. 21 is an image illustrating C1GALT1 expression in colorectal cancer tissues and non-tumor tissues analyzed by immunohistochemistry in accordance with an embodiment of the present invention.

FIG. 22 is a schematic diagram illustrating the comparison of the C1GALT1 immunohistochemical staining results between colorectal cancer tissues and non-tumor tissues in accordance with an embodiment of the present invention.

FIG. 23 is a graph illustrating the correlation between survival rates and the immunohistochemical staining results in colorectal cancer in accordance with an embodiment of the present invention.

FIG. 24 is an autoradiogram of the expression of C1GALT1 in 7 colorectal cancer cell lines analyzed by Western blotting in accordance with an embodiment of the present invention.

FIG. 25 is an autoradiogram illustrating the overexpression and knockdown of C1GALT1 in colon cancer cells confirmed by Western blotting in accordance with an embodiment of the present invention.

FIG. 26 is an image illustrating metastasis tumor nodules in lungs in accordance with an embodiment of the present invention.

FIG. 27 is a schematic diagram illustrating the total number of metastasis tumor nodules in accordance with an embodiment of the present invention.

FIG. 28 is a schematic diagram illustrating the number of tumors of colorectal cancer in a control group and a C1GALT1 knockdown group relative to days in accordance with an embodiment of the present invention.

FIG. 29 is a schematic diagram illustrating the tumor weight of colorectal cancer in a control group and the C1GALT1 knockdown group relative to days in accordance with an embodiment of the present invention.

FIGS. 30A-30B are schematic diagrams illustrating the effects of C1GALT1 on cell growth of colorectal cancer cells in accordance with an embodiment of the present invention.

FIG. 31 is a schematic diagram illustrating the effects of C1GALT1 on migration of colorectal cancer cells in accordance with an embodiment of the present invention.

FIG. 32 is a schematic diagram illustrating the effects of C1GALT1 on the invasion of colorectal cancer cells in accordance with an embodiment of the present invention.

FIG. 33 is a schematic diagram illustrating the effects of C1GALT1 on sphere formation of colorectal cancer cells in accordance with an embodiment of the present invention.

FIGS. 34-35 are schematic diagrams illustrating the effects of C1GALT1 on stem cell-like properties of colorectal cancer cells in accordance with an embodiment of the present invention.

FIGS. 36A-36B are autoradiograms illustrating the effects of C1GALT1 on the bFGF-induced signaling and EGF-induced signaling in sphere formation of colorectal cancer cells in accordance with an embodiment of the present invention.

FIGS. 37A-37B are autoradiograms illustrating the effects of C1GALT1 on the bFGF-induced signaling in migration and invasion of colorectal cancer cells in accordance with an embodiment of the present invention.

FIG. 38 is an autoradiogram illustrating the sialyl T and the sialyl Tn antigen decoration on FGFR2 in colorectal cancer cells in accordance with an embodiment of the present invention.

FIGS. 39A-39B are an autoradiogram illustrating the effects of C1GALT1 on the O-glycosylation of FGFR2 in colorectal cancer cells in accordance with an embodiment of the present invention.

FIG. 40 is an autoradiogram illustrating the effects of C1GALT1 on the tyrosine phosphorylation of FGFR2 in colorectal cancer cells in accordance with an embodiment of the present invention.

FIG. 41 is an image illustrating C1GALT1 immunohistochemical staining intensity scores of breast cancer tissue in accordance with an embodiment of the present invention.

FIGS. 42A-42B are schematic diagrams illustrating the C1GALT1 staining intensity scores against the histological grades and the tumor stages of breast cancer in accordance with an embodiment of the present invention.

FIG. 42C is a schematic diagram illustrating the C1GALT1 expression levels against the tumor stages of breast cancer in accordance with an embodiment of the present invention.

FIG. 43 is an autoradiogram illustrating C1GALT1 expression in six breast cancer cell lines analyzed by Western blotting in accordance with an embodiment of the present invention.

FIG. 44 is an autoradiogram illustrating C1GALT1 expression in six breast cancer cell lines analyzed by quantitative real-time RT-PCR (Q-PCR) in accordance with an embodiment of the present invention.

FIGS. 45A-45B are schematic diagrams illustrating C1GALT1 expression analyzed by quantitative real-time RT-PCR in breast cancer cell lines of a control group and a C1GALT1 knockdown group in accordance with an embodiment of the present invention.

FIG. 45C is a schematic diagram illustrating C1GALT1 expression analyzed by quantitative real-time RT-PCR in breast cancer cell lines of a control group and a C1GALT1 overexpression group in accordance with an embodiment of the present invention.

FIGS. 46A-46B are schematic diagrams illustrating C1GALT1 expression analyzed by Western blotting in breast cancer cell lines of a control group and a C1GALT1 knockdown group in accordance with an embodiment of the present invention.

FIG. 46C is a schematic diagram illustrating C1GALT1 expression analyzed by Western blotting in breast cancer cell lines of a control group and a C1GALT1 overexpression group in accordance with an embodiment of the present invention.

FIGS. 47A-47B are graphs illustrating the effects of C1GALT1 on the cell viability of breast cancer cells by MTT assay in accordance with an embodiment of the present invention.

FIGS. 48A-48C are schematic diagrams illustrating the effects of C1GALT1 on the migration of breast cancer cells in accordance with an embodiment of the present invention.

FIGS. 49A-49C are schematic diagrams illustrating the effects of C1GALT1 on the invasion of breast cancer cells in accordance with an embodiment of the present invention.

FIG. 50 is a schematic diagram illustrating the effects of C1GALT1 on stem-cell markers in breast cancer cells in accordance with an embodiment of the present invention.

FIG. 51 is an image illustrating the effects of C1GALT1 on sphere formation of breast cancer cells in accordance with an embodiment of the present invention.

FIG. 52 is a schematic diagram illustrating the effects of C1GALT1 on sphere formation of breast cancer cells in accordance with an embodiment of the present invention.

FIGS. 53A-53B are autoradiograms illustrating the effects of C1GALT1 on O-glycosylation of glycoproteins in breast cancer cell lines in accordance with an embodiment of the present invention.

FIGS. 54A-54B are graphs illustrating surface O-glycans of breast cancer cell surfaces analyzed by flow cytometry in accordance with an embodiment of the present invention.

FIGS. 55A-55E are autoradiograms illustrating the effects of C1GALT1 on O-glycosylation on MUCI in breast cancer cells in accordance with an embodiment of the present invention.

FIGS. 56A-56B include an image, a schematic diagram, and a graph illustrating the effects of C1GALT1 on the tumor growth of breast cancer cells in accordance with an embodiment of the present invention.

FIG. 57 is a schematic diagram illustrating mRNA expression of C1GALT1 in head and neck squamous cell carcinoma (HNSCC).

FIG. 58 is an autoradiogram illustrating C1GALT1 expression in three head and neck squamous cell carcinoma (HNSCC) cell lines analyzed by Western blotting in accordance with an embodiment of the present invention.

FIG. 59 is an autoradiogram illustrating C1GALT1 expression and the effects of C1GALT1 on O-glycosylation of glycoproteins in head and neck squamous cell carcinoma (HNSCC) of a control group and C1GALT1 knockdown groups in accordance with an embodiment of the present invention.

FIG. 60 is a graph illustrating surface O-glycans of HNSCC cell surfaces analyzed by flow cytometry in accordance with an embodiment of the present invention.

FIG. 61 is a schematic diagram illustrating the effect of C1GALT1 on the migration of HNSCC in accordance with an embodiment of the present invention.

FIG. 62 is a schematic diagram illustrating mRNA expression of C1GALT1 in lung adenocarcinoma and normal lung tissues.

FIG. 63 is a schematic diagram illustrating fold changes of mRNA expression of C1GALT1 in 8 different biosets.

FIG. 64 is an autoradiogram illustrating C1GALT1 expression in three lung cancer cell lines analyzed by Western blotting in accordance with an embodiment of the present invention.

FIG. 65 is an autoradiogram illustrating the overexpression and knockdown of C1GALT1 in lung cancer cell lines confirmed by Western blotting in accordance with an embodiment of the present invention.

FIG. 66 is a schematic diagram illustrating C1GALT1 overexpression enhancing sphere formation in lung cancer cells in accordance with an embodiment of the present invention.

FIGS. 67A-67B are schematic diagrams illustrating the effects of C1GALT1 on the migration of lung cancer cells in accordance with an embodiment of the present invention.

FIGS. 68A-68B are schematic diagrams illustrating the effects of C1GALT1 on the invasion of lung cancer cells in accordance with an embodiment of the present invention.

FIG. 69 is a schematic diagram illustrating mRNA expression of C1GALT1 in various types ovarian cancers and normal ovarian tissues.

FIG. 70 is an autoradiogram illustrating C1GALT1 expression in three ovarian cancer cell lines analyzed by Western blotting in accordance with an embodiment of the present invention.

FIG. 71 is an autoradiogram illustrating knockdown of C1GALT1 in an ovarian cancer cell line confirmed by Western blotting in accordance with an embodiment of the present invention.

FIG. 72 is a schematic diagram illustrating the effects of C1GALT1 on the cell growth of ovarian cancer cells in accordance with an embodiment of the present invention.

FIG. 73 is a schematic diagram illustrating the effects of C1GALT1 on the migration of ovarian cancer cells in accordance with an embodiment of the present invention.

FIG. 74 is a schematic diagram illustrating the effects of C1GALT1 on the invasion of ovarian cancer cells in accordance with an embodiment of the present invention.

FIG. 75 is a schematic diagram illustrating mRNA expression of C1GALT1 in fold changes in 3 different biosets.

FIG. 76 is an autoradiogram illustrating C1GALT1 expression in two endometrial cancer cell lines analyzed by Western blotting in accordance with an embodiment of the present invention.

FIG. 77 is an autoradiogram illustrating knockdown of C1GALT1 in an endometrial cancer cell line confirmed by Western blotting in accordance with an embodiment of the present invention.

FIG. 78 is a schematic diagram illustrating the effects of C1GALT1 on the migration of endometrial cancer cells in accordance with an embodiment of the present invention.

FIG. 79 is a schematic diagram illustrating the effects of C1GALT1 on the invasion of endometrial cancer cells in accordance with an embodiment of the present invention.

FIG. 80A is a bile duct tissue image illustrating different intensities of C1GALT1 staining in accordance with an embodiment of the present invention.

FIG. 80B is a schematic diagram illustrating percentages of cases with different intensity levels of C1GALT1 immunohistochemistry staining in cholangiocarcinoma (CCA) and bile duct tissues in accordance with an embodiment of the present invention.

FIG. 81 is an autoradiogram illustrating C1GALT1 expression in three CCA cell lines analyzed by Western blotting in accordance with an embodiment of the present invention.

FIG. 82 is an autoradiogram illustrating knockdown of C1GALT1 in two CCA cell lines confirmed by Western blotting in accordance with an embodiment of the present invention.

FIGS. 83A-83B are schematic diagrams illustrating the effects of C1GALT1 on the migration of CCA cells in accordance with an embodiment of the present invention.

FIGS. 84A-84B are schematic diagrams illustrating the effects of C1GALT1 on the invasion of CCA cells in accordance with an embodiment of the present invention.

FIGS. 85A-85D are graphs illustrating that itraconazole impairs C1GALT1 function leading to accumulation of Tn antigen expression in lung cancer cells in accordance with an embodiment of the present invention.

FIGS. 86A-86D are graphs illustrating that itraconazole impairs C1GALT1 function leading to accumulation of Tn antigen expression in breast cancer cells in accordance with an embodiment of the present invention.

FIGS. 87A-87D are graphs illustrating that itraconazole impairs C1GALT1 function leading to accumulation of Tn antigen expression in liver cancer cells in accordance with an embodiment of the present invention.

FIGS. 88A-88C are schematic diagrams respectively illustrating that itraconazole suppresses lung, breast, and liver cancer cell growth in accordance with an embodiment of the present invention.

### DETAILED DESCRIPTION OF THE PREFERRED EMBODIMENTS

By reference to the accompanying drawings, the specific embodiments of the methods for treating a cell proliferative disorder by inhibiting C1GALT1 expression or activity are described in detail as follows:

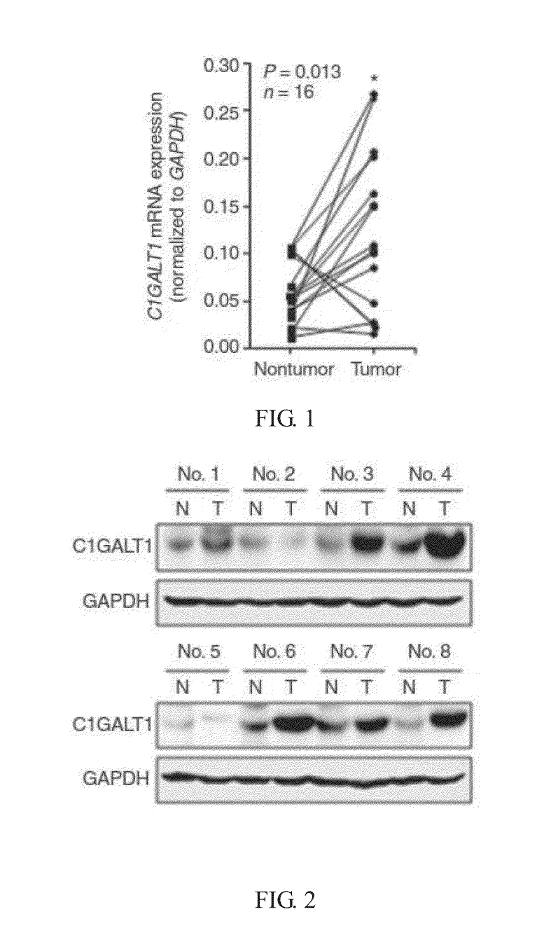

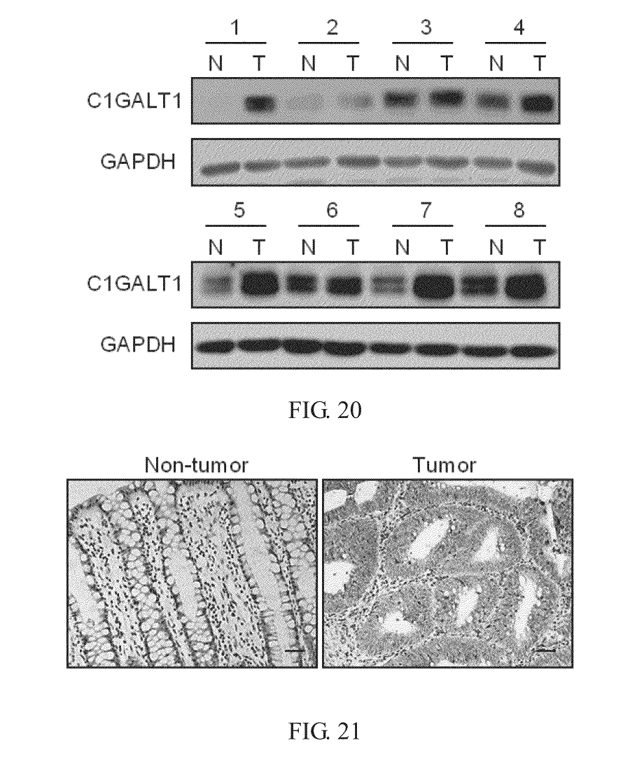

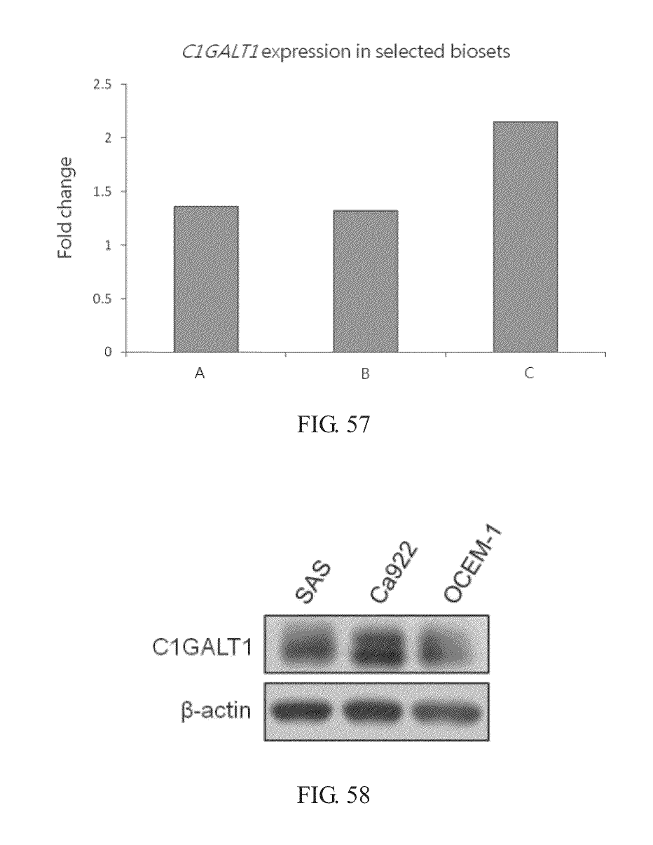

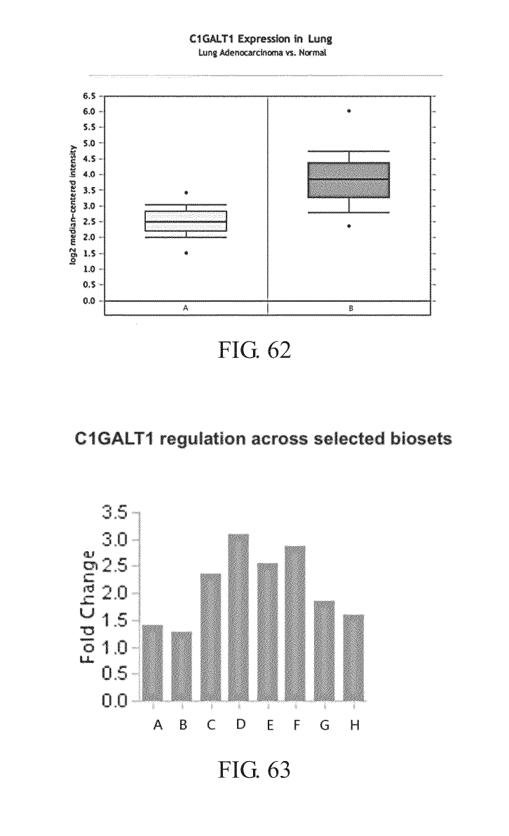

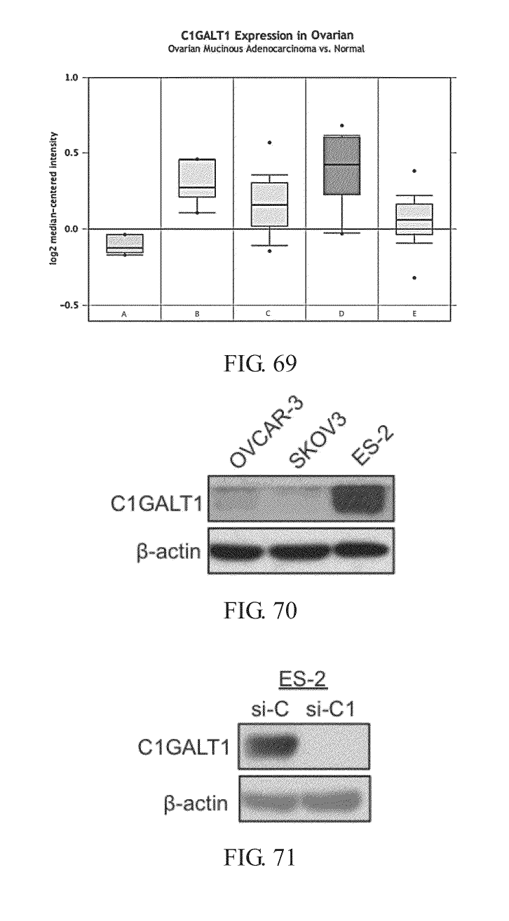

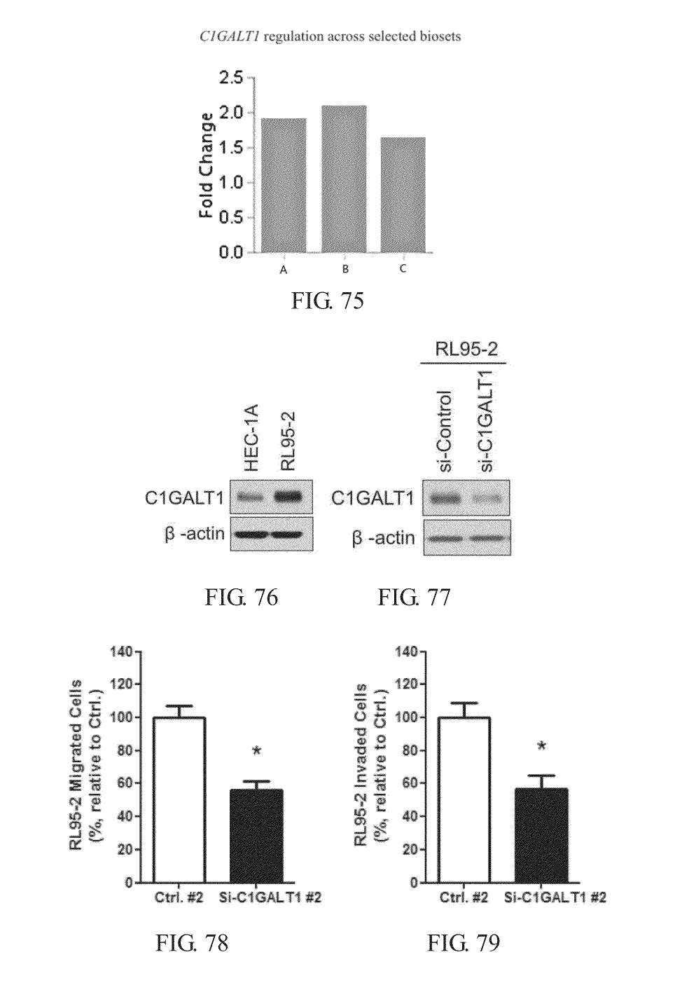

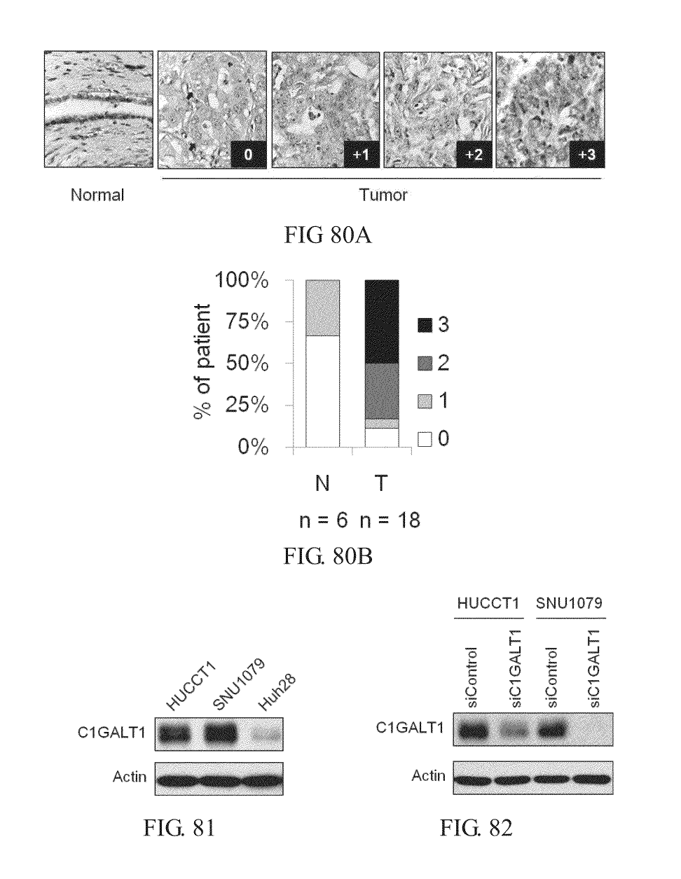

The cell proliferative disorder in the present invention refers to any disturbance or derangement related to cell proliferation in a body, including abnormal cell proliferation, cell migration, cell invasion, benign tumor growth, malignant tumor growth and malignant tumor metastasis. Referring to FIGS. 1-19, FIGS. 87A-87D, and FIG. 88C, the embodiments of the present invention provide a method for treating hepatocellular carcinoma. Referring to FIGS. 20-39, the embodiments of the present invention provide a method for treating colorectal cancer. Referring to FIGS. 40-56, FIGS. 86A-86D and FIG. 88B, the embodiments of the present invention provide a method for treating breast cancer. Referring to FIGS. 57-61, the embodiments of the present invention provide a method for treating head and neck squamous cancer. Referring to FIGS. 62-68, FIGS. 85A-85D, and FIG. 88A, the embodiments of the present invention provide a method for treating lung cancer. Referring to FIGS. 69-74, the embodiments of the present invention provide a method for treating ovarian cancer. Referring to FIGS. 75-79, the embodiments of the present invention provide a method for treating endometrial cancer. Referring to FIGS. 80-84, the embodiments of the present invention provide a method for treating cholangiocarcinoma cancer, also known as bile duct cancer. In these methods, a C1GALT1 inhibition substance is administered to inhibit C1GALT1 expression or activity in human cancer cells. The C1GALT1 inhibition substance includes any substance which can inhibit the C1GALT1 expression or activity, such as an antisense nucleotide (siRNA or shRNA), a small molecular substance (an organic compound whose molecular weight is less than 900 kD), or a large molecule substance (a protein, an antibody, an enzyme, or biologics). In the embodiments of the present invention, siRNA, shRNA plasmid, and itraconazole, which is a triazole small molecule drug, are taken as examples of the C1GALT1 inhibition substance in the experiments in vitro, and shRNA plasmid is taken as an example of the C1GALT1 inhibition substance in the experiments in vivo.

### Hepatocellular Carcinoma

Hepatocellular carcinoma is the fifth most common solid tumor and the third leading cause of cancer related deaths worldwide. Because of late-stage diagnosis and limited therapeutic options, the prognosis of patients with hepatocellular carcinoma after medical treatments remains disappointing. Diverse posttranslational modifications control various properties of proteins and correlate with many diseases, including cancer. Although comprehensive genomic and proteomic analyses have identified many key drivers of hepatocellular carcinoma, the posttranslational modifications remain poorly understood. Thus, elucidation of the precise molecular mechanisms underlying hepatocellular carcinoma progression is of great importance for developing new reagents to treat this aggressive disease.

The methods in accordance with hepatocellular carcinoma embodiments of the present invention are described as follows. However, the following materials and specific procedures provided are illustrative only and not intended to be limiting.

### Hepatocellular Carcinoma Resource and Cell Culture Conditions

Postsurgery frozen hepatocellular carcinoma tissues for RNA extraction and Western blotting and paraffin-embedded tissue sections are obtained from the National Taiwan University Hospital (Taipei, Taiwan). This study is approved by the Ethical Committees of National Taiwan University Hospital, and all patient s gave informed consent to have their tissues before collection.

Human liver cancer cell lines, Huh7, PLC5, Sk-Hep1, and HepG2, are purchased from Bioresource Collection and Research Center in the year 2008. HA22T, SNU387, and HCC36 cells are kindly provided by Prof. Shiou-Hwei Yeh (National Taiwan University) in the year 2010. All cell lines are authenticated by the provider based on morphology, antigen expression, growth, DNA profile, and cytogenetics. Cells are cultured in Dulbecco's Modified Eagle Medium (DMEM) containing 10% FBS in 5% CO2 at 37° C. To analyze growth factor-induced cell signaling, cells are starved in serum-free DMEM for 5 hours and then treated with 25 ng/mL of hepatocyte growth factor (HGF) or insulin-like growth factor (IGF) at 37° C. for 30 minutes.

### Methods of Inhibition of C1GALT1 Expression and Activity in Hepatocellular Carcinoma Cells

According to an embodiment of the present invention, in the experiment in vitro, two siRNA oligonucleotides against C1GALT1, 5′-UUAGUAUACGUUCAGGUAAGGUA GG-3′ (SEQ ID NO: 1) and 5′-UUAUGUUGGCUAGAAUCUGCAUUGA-3′ (SEQ ID NO: 2), used as the C1GALT1 inhibition substance and a negative control siRNA of medium GC are synthesized by Invitrogen company. The negative control siRNA oligonucleotides, 5′-UAAAUGUACUGCGCGUGGAGAGGAA-3′ (SEQ ID NO: 3), do not target mRNA of any gene. For knockdown of C1GALT1, cells are transfected with 20 nM of siRNA using Lipofectamine® RNAiMAX (Invitrogen) for 48 hours.

According to another embodiment of the present invention, in the experiment in vitro, itraconazole, a small molecular drug, is used as the C1GALT1 inhibition substance. Itraconazole is an FDA approved drug that is clinically employed as an antimicrobial for the treatment of fungal, mycosis, and candida infections. The inventors of the present invention discovered that the structure of itraconazole is suitable for binding to the catalytic domain of C1GALT1 and inhibiting C1GALT1. Cells are cultured in DMEM with 10% FBS and treated with itraconazole 10 ug/ml for 3 days at 37° C. under 5% CO₂.

According to another embodiment of the present invention, in the experiment in vivo, the pLKO/C1GALT1-shRNA plasmid containing the sequence, 5′-CCCAGCCTAATG TTCTTCATA-3′(SEQ ID NO: 13), against C1GALT1 is used as the C1GALT1 inhibition substance. The pLKO/C1GALT1-shRNA plasmid and non-targeting pLKO plasmids (as a negative control) are purchased from National RNAi Core Facility (Academia Sinica, Taipei, Taiwan). The short hairpin RNA (shRNA, 20 nM) plasmids are transfected with Lipofectamine® 2000 and selected with 500 ng/mL of puromycin for 10 days. Knockdown of C1GALT1 in single colonies is confirmed by Western blotting. Subsequently, 7×10⁶ of hepatocellular carcinoma cells containing the pLKO/C1GALT1-shRNA plasmid and the non-targeting pLKO plasmid are subcutaneously injected into severe combined immunodeficient (SCID) mice (n=4 for each group). Tumor volumes are monitored for 36 or days. Excised tumors are weighed and lysed for Western blotting and immunohistochemistry.

### Methods for Overexpressing C1GALT1 in Liver Cancer Cells

To overexpress C1GALT1, cells are transfected with pcDNA3.1/C1GALT1/mycHis plasmids, which contains a promoter and cDNA of C1GALT1, shown as SEQ ID NO: 4 in the sequence list, by using Lipofectamine® 2000 (Invitrogen) according to the manufacturer's protocols. Empty pcDNA3.1/mycHis plasmid is used as mock transfectant. The transfected cells are selected with 600 mg/mL of G418 for 14 days and then pooled for further studies.

### Analysis Procedures of Effects of C1GALT1 Inhibition on Hepatocellular Carcinoma

### Tissue Array and Immunohistochemistry:

Antibodies against C1GALT1 are purchased from Santa Cruz Biotechnology, Inc. Paraffin-embedded human hepatocellular carcinoma tissue microarrays are purchased from SuperBioChips and BioMax. Arrays are incubated with anti-C1GALT1 monoclonal antibody (1:200) in 5% bovine serum albumin/PBS and 0.1% Triton X-100 (Sigma) for 16 hours at 4° C. After rinsing twice with PBS, SuperSensitive Link-Label IHC Detection System (BioGenex) is used and the specific immunostaining is visualized with 3,3-diaminobenzidine liquid substrate system (Sigma). All sections are counterstained with hematoxylin (Sigma). Western blotting:

Antibodies against C1GALT1, glyceraldehyde-3-phosphate dehydrogenase (GAPDH), and IGF-IR (receptor) are purchased from Santa Cruz Biotechnology, Inc. Antibodies against MET pY 1234/5, IGF-IR pY1135/1136, p-AKT, p-ERK1/2, and ERK1/2 are purchased from Cell Signaling Technology, Inc. Antibodies against total MET and AKT are purchased from GeneTex, Inc.

Equal amounts of protein samples are mixed with 5× sample buffer and boiled for 5 minutes, separated on SDS-polyacrylamide gels, and then transferred to PVDF membrane. The membranes are blocked in 5% BSA for 1 hour at room temperature, and incubated with primary antibodies overnight at 4° C. Anti-phosphotyrosine (4G10) antibody (Millipore, Billerica, Mass.), antibodies against C1GALT1, GAPDH, IGF-IR, MET pY 1234/5, IGF-IR pY1135/1136, p-AKT, p-ERK1/2, and ERK1/2 antibody (BD Pharmingen, San Jose, Calif.) are used. The blots are then incubated with secondary antibody conjugated with horseradish peroxidase and immunoreacted bands are detected by ECL reagents and exposed on x-ray film.

cDNA Synthesis and Quantitative Real-Time RT-PCR (Q-PCR):

Total RNA is isolated using TRIzol reagent (Invitrogen) according to the manufacturer's protocol. Two micrograms of total RNA is used in reverse transcription reaction using the Superscript III First-Strand cDNA Synthesis Kit (Invitrogen). The cDNA is subjected to quantitative real-time RT-PCR (Q-PCR). Quantitative PCR System Mx3000P (Stratagene, La Jolla, Calif.) is used for quantitative real-time RT-PCR. Reactions are performed in a 20 μl volume with 2 μl cDNA, 400 nM of sense and anti-sense primers, and 10 μl Brilliant®SYBR®Green Q-PCR Master Mix (Stratagene). Primer sets are designed as the following: Primers for C1GALT1 are 5′-TGGGAGAAAAGGTTGACACC-3′ (SEQ ID NO: 5) and 5′-CTTTGACGTGTTTGGCCTTT-3′(SEQ ID NO: 6). Primers for GAPDH are 5 ‘-GACAAGCTTCCCGTTCTCAG-3’ (SEQ ID NO: 8) and 5′-ACAGTCAGCCGCATCTTCTT-3′ (SEQ ID NO: 7). The relative quantity of gene expression normalized to GAPDH is analyzed using MxPro Software (Stratagene).

### Phospho-Receptor Tyrosine Kinase Array:

A human phospho-receptor tyrosine kinase (p-RTK) array kit is purchased from R&D systems. Hepatocellular carcinoma cells are serum starved for 5 hours and then treated with 20% FBS for 30 minutes. Cells are lysed and 500 mg of proteins are subjected to Western blotting according to the manufacturer's protocol.

### Cell Viability, Proliferation, and Cell Cycle Analyses:

Cells (4×10⁴) are seeded in each well of 6-well plates with DMEM containing 10% FBS. Viable cells are analyzed by Trypan blue exclusion assay at 0, 24, 48, and 72 hours. Cell proliferation is evaluated by immunostaining with anti-Ki67 antibody (1:1000; Vector Laboratories). For cell-cycle analysis, 1×10⁶ cells are stained with propidium iodide (Sigma) for 30 minutes. The percentages of cells in G1, S, and G2M phases are analyzed by flow cytometry (Becton Dickinson).

### Deglycosylation and Lectin Pull Down:

Vicia villosa agglutinin (VVA)- and peanut agglutinin (PNA) lectin-conjugated agarose beads, fluorescein isothiocyanate (FITC)-, and biotinylated VVA were purchased from Vector Laboratories. Recombinant EGF, HGF, IGF-I, and protein deglycosylation kits were purchased from Sigma. Protein deglycosylation is carried out using an Enzymatic Protein Deglycosylation Kit (Sigma). Briefly, cell lysates are treated with neuraminidase or PNGase F at 37° C. for 1 hour. For lectin blotting, 20 mg of cell lysate is separated by an 8% SDS-PAGE, transferred to polyvinylidene difluoride membrane (Millipore), and blotted with biotinylated VVA (1:10,000). For lectin pull-down assay, cell lysates (0.3 mg) are incubated with or without deglycosylation enzymes and then applied to VVA- or PNA-conjugated agarose beads at 4° C. for 16 hours. The pulled down proteins are analyzed by Western blotting.

### Analysis of Dimerization of MET:

MET is also called hepatocyte growth factor receptor (HGFR). The MET gene is an important protooncogene in a variety of human cancers. To analyze dimerization of MET, hepatocellular carcinoma cells are incubated with or without 25 ng/mL of human HGF in DMEM on ice for 5 minutes. Cross-linker Bis(sulfosuccinimidyl) suberate (BS3, 0.25 mmol/L, Thermo Scientific) is added to cells and reacted at 37° C. for 5 minutes. Cells are then transferred on ice for 10 minutes. Reactions are blocked by adding 50 mmol/L of Tris-HCl (pH 7.4). Cell lysates are separated by 6% SDS-PAGE and immunoblotted with anti-MET antibody.

### Statistical Analysis:

Student t test is used for statistical analyses. Paired t test is used for the analyses of paired hepatocellular carcinoma tissues. Mann-Whitney U test is used to compare unpaired non-tumor liver tissue and hepatocellular carcinoma tissues. Two-sided Fisher exact test is used for comparisons between C1GALT1 expression and clinicopathologic features. Kaplan-Meier analysis and the log-rank test are used to estimate overall survival. Pearson correlation test is used to assess C1GALT1 and phosphorylation of MET (p-MET) expression. Data are presented as means±SD. P<0.05 is considered statistically significant.

### Analysis Results of Effects of C1GALT1 on Hepatocellular Carcinoma

Expression of C1GALT1 is Upregulated in Hepatocellular Carcinoma and Correlates with Advanced Tumor Stage, Metastasis, and Poor Survival:

Firstly, expression of C1GALT1 in hepatocellular carcinoma tissues was investigated. FIG. 1 is a schematic diagram of illustrating mRNA expression of C1GALT1 in paired hepatocyte carcinoma tissues and non-tumor liver tissues analyzed by quantitative real-time RT-PCR in accordance with an embodiment of the present invention. Expression of C1GALT1 mRNA in 16 paired hepatocellular carcinoma tissues was investigated. The mRNA levels of C1GALT1 were analyzed by quantitative real-time RT-PCR and normalized to GAPDH. (Paired t test, *, P=0.013). Paired hepatocellular carcinoma and adjacent non-tumor liver tissues (n=16) were analyzed. Results showed that C1GALT1 mRNA was significantly upregulated in hepatocellular carcinoma tissues compared with adjacent non-tumor liver tissues, as shown in FIG. 1.

FIG. 2 is an autoradiogram illustrating C1GALT1 expression in paired hepatocyte carcinoma tissues (T) and non-tumor liver tissues (N) analyzed by Western blotting in accordance with an embodiment of the present invention. Western blot analyses showing C1GALT1 expression in paired hepatocellular carcinoma tissues from FIG. 1. N refers to non-tumor liver tissues. T refers to tumor tissue. Consistently, Western blotting showed that C1GALT1 protein is overexpressed in hepatocellular carcinoma tissues of paired specimens, as shown in FIG. 2. Immunohistochemical analysis was conducted for 72 primary hepatocellular carcinoma tissues and 32 non-tumor livers to investigate the expression of C1GALT1.

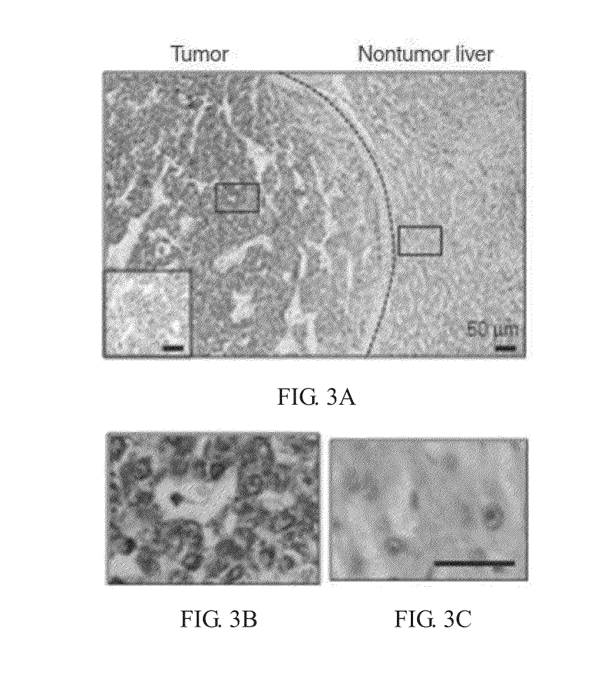

FIG. 3A is an image illustrating C1GALT1 expression in hepatocellular carcinoma tissues and non-tumor liver tissues analyzed by immunohistochemistry in accordance with an embodiment of the present invention. FIGS. 3B-3C are amplified images of regions of FIG. 3A. The brown stained cells in the tumor part are hepatocellular carcinoma cells, and those in the non-tumor part are hepatocytes. The staining was visualized with a 3,3-diaminobenzidine liquid substrate system, and all sections were counterstained with hematoxylin. Representative images of tumor at left of FIG. 3A and non-tumor liver tissue are shown at right of FIG. 3A. The negative control does not show any specific signals at bottom left of FIG. 3A. The scale bars are 50 mm. The immunohistochemistry showed dot-like precipitates of C1GALT1 in the cytoplasm of hepatocellular carcinoma, as shown in FIGS. 3A-3B, which is similar to the intracellular localization of the Golgi apparatus in hepatocytes. Expression of C1GALT1 in surrounding stromal cells was not observed under our experimental conditions.

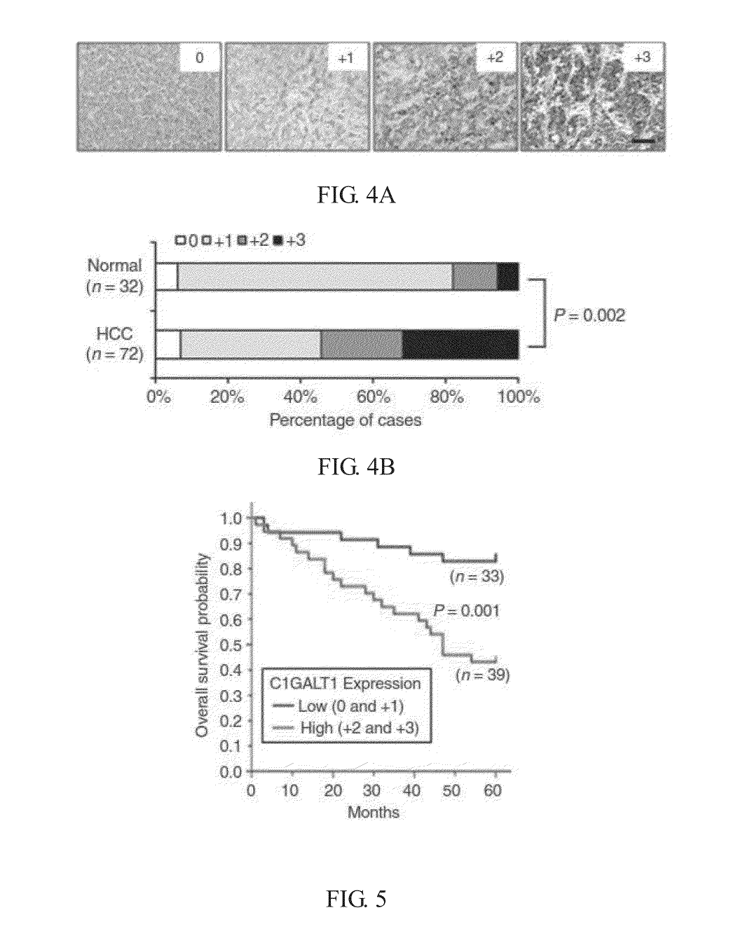

FIG. 4A is a tissue image illustrating different intensities of C1GALT1 staining in accordance with an embodiment of the present invention. FIG. 4B is a schematic diagram illustrating percentages of cases with different intensity levels of C1GALT1 immunohistochemistry staining in hepatocellular carcinoma and hepatocytes in accordance with an embodiment of the present invention Immunohistochemistry staining results in hepatocellular carcinoma tissues were analyzed statistically. The scale bars are 50 mm. The results are shown in FIG. 4B (Mann-Whitney U Test, P=0.002). The intensity of staining was scored according to the percentage of C1GALT1-positive cells in each sample (0, negative; +1, <20%; +2, 20%-50%; +3, >50%). The data revealed that C1GALT1 was highly expressed (+2 and +3) in 54% of hepatocellular carcinoma tumors (HCC), whereas only 19% of non-tumor liver tissues (or normal tissues) expressed high levels of C1GALT1 (Mann-Whitney U Test, P=0.002), as shown in FIG. 3, FIG. 4A, and FIG. 4B. Consistently, results from tissue microarrays also showed increased expression of C1GALT1 in hepatocellular carcinomas compared with normal liver tissues (not shown). These results indicated that C1GALT1 expression was significantly higher in hepatocellular carcinoma than that in non-tumor liver tissues.

FIG. 5 is a schematic diagram illustrating overall survival probabilities for patients with hepatocellular carcinoma with high and low C1GALT1 expression in accordance with an embodiment of the present invention. The analyses were conducted according to the immunohistochemistry results of C1GALT1, as shown in FIGS. 4A-4B. Probability of overall survival was analyzed after the initial tumor resection (Log-rank test, P=0.001). The relationship between C1GALT1 expression and clinicopathologic features in patients with hepatocellular carcinoma was investigated. High expression of C1GALT1 correlated with advanced tumor stage (Fisher exact test, P<0.05) and metastasis (Fisher exact test, P<0.01) of hepatocellular carcinoma tumors was found, as shown in Table 1 below. A Kaplan-Meier survival analysis showed that the survival rate of patients with hepatocellular carcinoma with high C1GALT1 expression was significantly lower than those with low C1GALT1 expression. (log-rank test, P=0.001), as shown in FIG. 5. Collectively, these data suggest that C1GALT1 was frequently upregulated in hepatocellular carcinoma and its expression was associated with advanced tumor stage, metastasis, and poor survival in hepatocellular carcinoma.

TABLE 1
Correlation of C1GALT 1 expression with clinicopathologic
features in hepatocellular carcinoma:

| | | C1GALT1 | | | |
|---|---|---|---|---|---|
| | | expression | | | |
| | | Low | High | | |
| Factor | | (n = 33) | (n = 39) | P | Method |
| Sex | Male | 27 | 26 | 0.185 | Two-sided |
| | Female | 6 | 13 | | Fisher exact |
| | | | | | test |
| Age, y | <66 | 11 | 9 | 0.430 | |
| | ≧55 | 22 | 30 | | |
| Histology grade | 1 + 2 | 18 | 28 | 0.147 | |
| | 3 + 4 | 15 | 11 | | |
| Tumor stage | T1 + T2 | 27 | 20 | 0.025^{a} | |
| | T3 + T4 | 6 | 19 | | |
| Metastasis | No | 33 | 30 | 0.003^{a} | |
| | Yes | 0 | 9 | | |
| Overall survival | | | | 0.001^{a} | Log rank |

^{a}P < 0.05 is considered statistically significant.

### C1GALT1 Modifies Mucin-Type O-Glycans on Hepatocellular Carcinoma Cells:

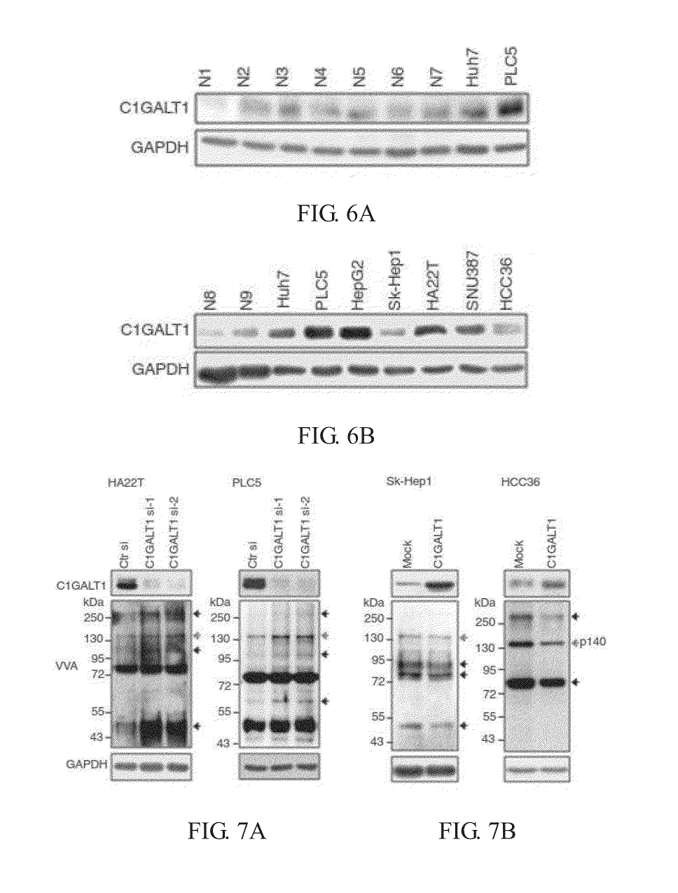

FIG. 6A is an autoradiogram illustrating C1GALT1 expression in seven hepatocyte carcinoma cell lines (Huh7, PLC5, HepG2, Sk-Hep1, HA22T, SNU387, and HCC36) and nine non-tumor liver tissues (N1-N9) analyzed by Western blotting in accordance with an embodiment of the present invention. FIG. 6B is a schematic diagram illustrating the quantified C1GALT1 expression signals from FIG. 6A in accordance with an embodiment of the present invention. GAPDH was used as an internal control. Signals were quantified by ImageQuant5.1. *, P<0.05. To investigate functions of C1GALT1 in hepatocellular carcinoma, knockdown or overexpression of C1GALT1 was conducted in multiple hepatocellular carcinoma cell lines. Western blotting showed that the average expression level of C1GALT1 was significantly higher in hepatocellular carcinoma cell lines compared with that of non-tumor liver tissues as shown in FIGS. 6A-6B.

FIG. 7A is an autoradiogram illustrating C1GALT1 expression and the effects of C1GALT1 on O-glycosylation of glycoproteins in hepatocellular carcinoma cell lines of a control group (Ctr si) and C1GALT1 knockdown groups (C1GALT1 si-1, C1GALT1 si-2) in accordance with an embodiment of the present invention. FIG. 7B is an autoradiogram illustrating C1GALT1 expression and the effects of C1GALT1 on O-glycosylation of glycoproteins in hepatocellular carcinoma cell lines of a control group (Mock) and a C1GALT1 overexpression group (C1GALT1) in accordance with an embodiment of the present invention. HA22T and PLC5 cells express high levels of C1GALT1, while Sk-Hep1 and HCC36 cells express low levels of C1GALT1, as shown in FIG. 6B. Compared with control (Ctr), knockdown of C1GALT1 in HA22T and PLC5 cells transfected with two different C1GALT1-specific siRNAs (C1GALT1 si-1, C1GALT1 si-2) is shown in FIG. 7A. Compared with pcDNA3.1 empty plasmid (mock), overexpression of C1GALT1 in Sk-Hep1 and HCC36 cells transfected with C1GALT1/pcDNA3.1 plasmid (C1GALT1) is shown in FIG. 7B Immunofluorescence microscopy further confirmed the knockdown and overexpression of C1GALT1 in hepatocellular carcinoma cells and its subcellular localization in the Golgi apparatus (not shown). Furthermore, the changes in O-glycans on glycoproteins were revealed by Western blotting with biotinylated VVA. Proteins with evident changes in VVA binding are indicated by arrows. p140 changes in all tested cell lines are indicated by red arrows. Knockdown of C1GALT1 enhanced binding of VVA to glycoproteins as shown in FIG. 7A, whereas overexpression of C1GALT1 decreased the VVA binding as shown in FIG. 7B, indicating that C1GALT1 catalyzes the formation of Tn to T antigen. Seven proteins had evident changes in VVA binding, including p50, p60, p80, p90, p110, p140, and p260, as shown in FIG. 7. Among them, p140 showed changes in all four tested cell lines.

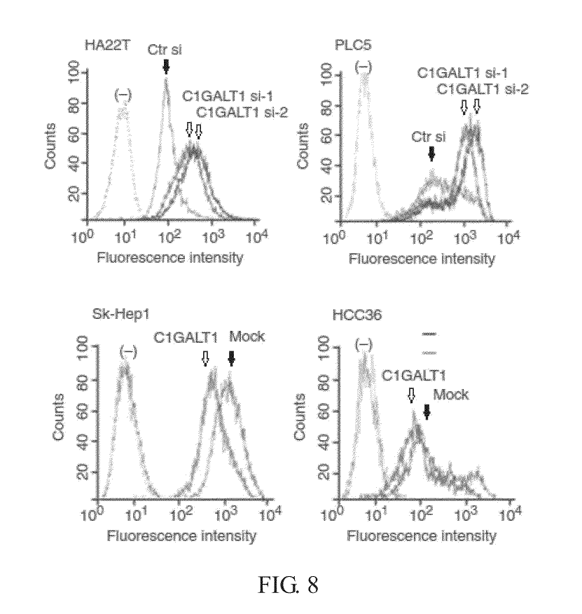

FIG. 8 is a graph illustrating surface O-glycans of hepatocellular carcinoma cell surfaces analyzed by flow cytometry with FITC-VVA in accordance with an embodiment of the present invention. Negative (−) refers to cells without the addition of VVA-FITC. Consistently, flow cytometry showed that C1GALT1 altered VVA binding to the surface of hepatocellular carcinoma cells, as shown in FIG. 8. These results indicate that C1GALT1 modulates the expression of mucin-type O-glycans on hepatocellular carcinoma cells.

### C1GALT1 Regulates Hepatocellular Carcinoma Cell Proliferation In Vitro and In Vivo:

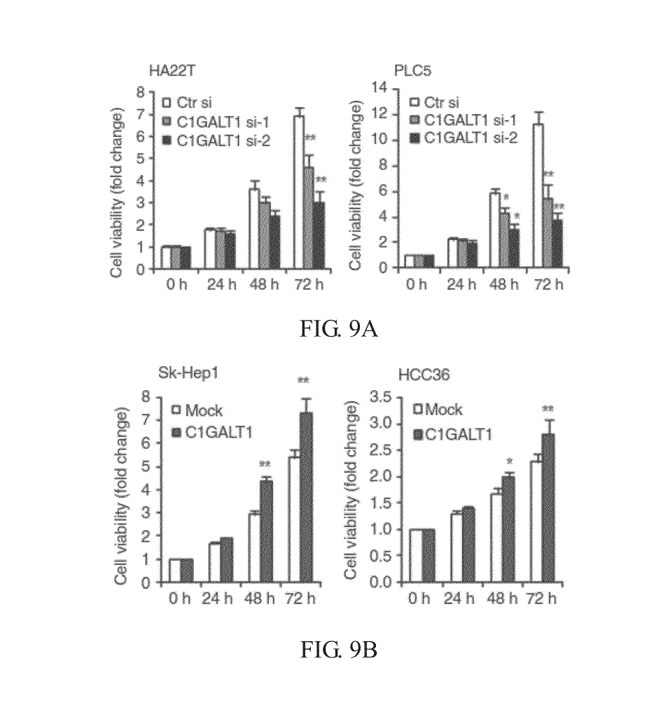

FIGS. 9A-9B are schematic diagrams illustrating the effects of C1GALT1 on the cell viability of hepatocellular carcinoma cells in vitro in accordance with an embodiment of the present invention. Cell viability of HA22T, PLC5, Sk-Hep1, and HCC36 cells was analyzed by Trypan blue exclusion assays at different time points for 72 hours. The results were standardized by the cell number at 0 hour. Data are represented as means±SD from three independent experiments. *, P<0.05; **, P<0.01. Knockdown of C1GALT1 (C1GALT1 si-1, C1GALT1 si-2) significantly suppressed cell viability, as shown in FIG. 9A, whereas overexpression of C1GALT1 enhanced cell viability, as shown in FIG. 9B.

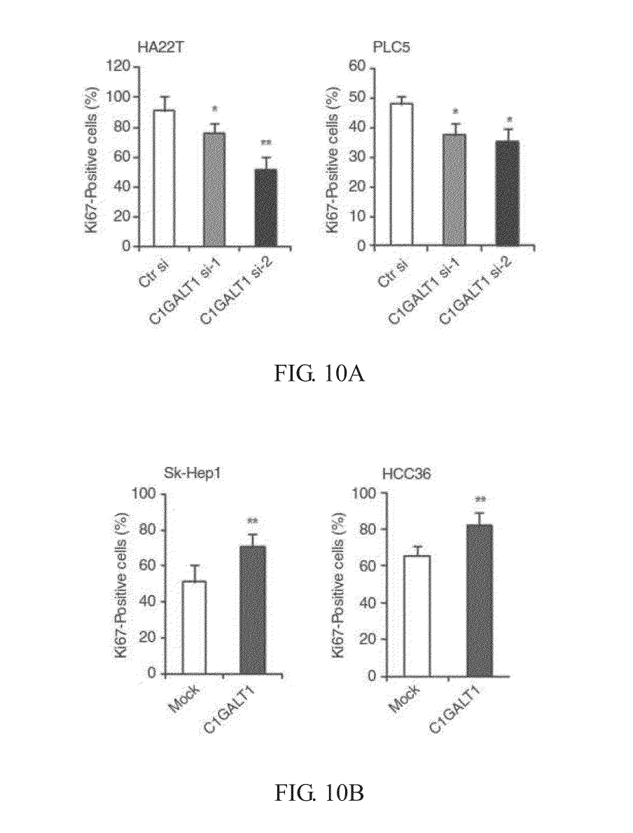

FIGS. 10A-10B are schematic diagrams illustrating the effects of C1GALT1 on cell proliferation of hepatocellular carcinoma cells in vitro in accordance with an embodiment of the present invention. Cells were immunofluorescently stained for Ki67 and Ki67-positive cells were counted under a microscope. Results are presented as means±SD from three independent experiments. *, P<0.05; **, P<0.01. Ki67 staining showed that C1GALT1 modulated cell proliferation as shown in FIGS. 10A-10B. Knockdown of C1GALT1 (C1GALT1 si-1, C1GALT1 si-2) led to G1-phase arrest in HA22T and PLC5 cells, whereas overexpression of C1GALT1 increased the number of cells in S phase in Sk-Hep1 cells (not shown).

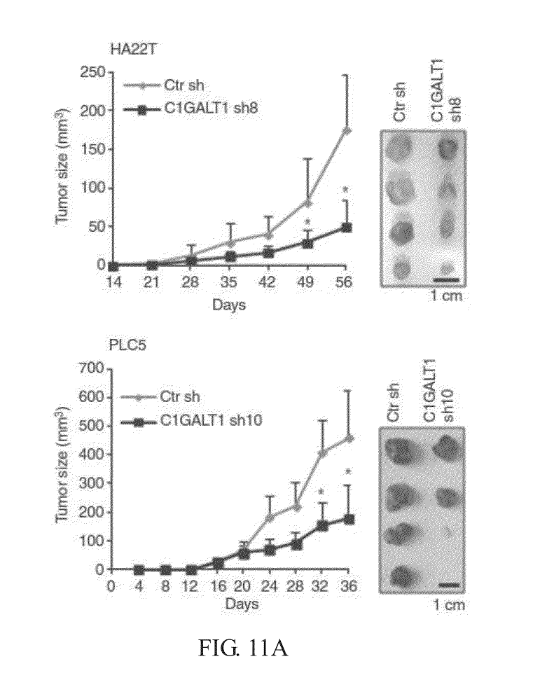

FIGS. 11A-11C are schematic diagrams and images illustrating the effects of C1GALT1 on tumor growth of hepatocellular carcinoma in a SCID mouse model in vivo in accordance with an embodiment of the present invention. To analyze the effect of C1GALT1 on tumor growth in vivo, C1GALT1 was stably knocked down with C1GALT1-specific shRNA in HA22T and PLC5 cells (C1GALT1 sh8, C1GALT1 sh10). Clone number 8 of HA22T cells (C1GALT1 sh8, top) and clone number 10 of PLC5 cells (C1GALT1 sh10, bottom) were subcutaneously xenografted in SCID mice. Four mice were used for each group. The sizes of tumors were measured at different time points, as indicated in FIG. 11A. Mice were sacrificed at day 56 for HA22T cells and day 36 for PLC5 cells. Tumors were excised and weighted, as shown in FIG. 11B. Cell proliferation of tumor cells was evaluated by immunohistochemistry, as shown in FIG. 11C. The results showed that knockdown of C1GALT1 significantly suppressed the volume and weight of hepatocellular carcinoma tumors Immunohistochemistry of excised tumors for Ki67 expression revealed that knockdown of C1GALT1 suppressed tumor cell proliferation in vivo, as shown in FIGS. 11A-11C. Knockdown of the C1GALT1 in the tumors was confirmed by Western blotting (not shown). These data provide evidence that C1GALT1 can modulate hepatocellular carcinoma cell proliferation in vitro and in vivo.

### C1GALT1 Regulates Phosphorylation of MET:

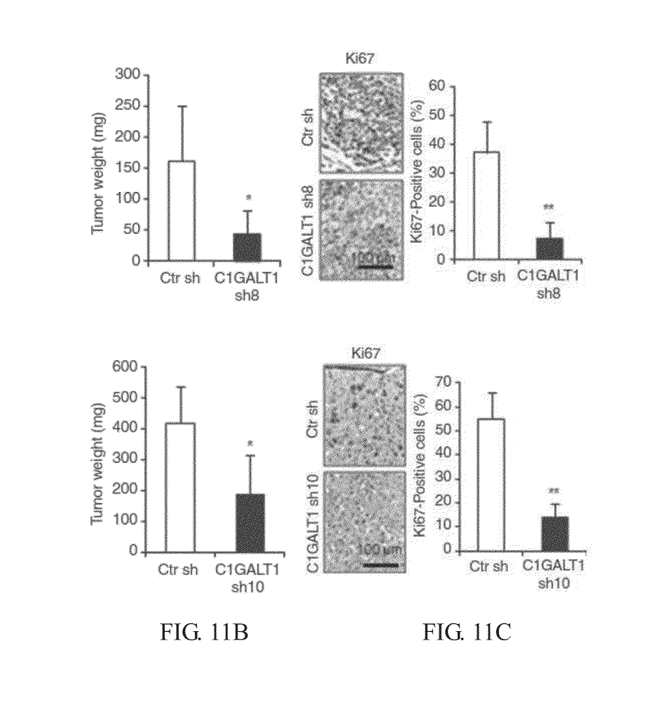

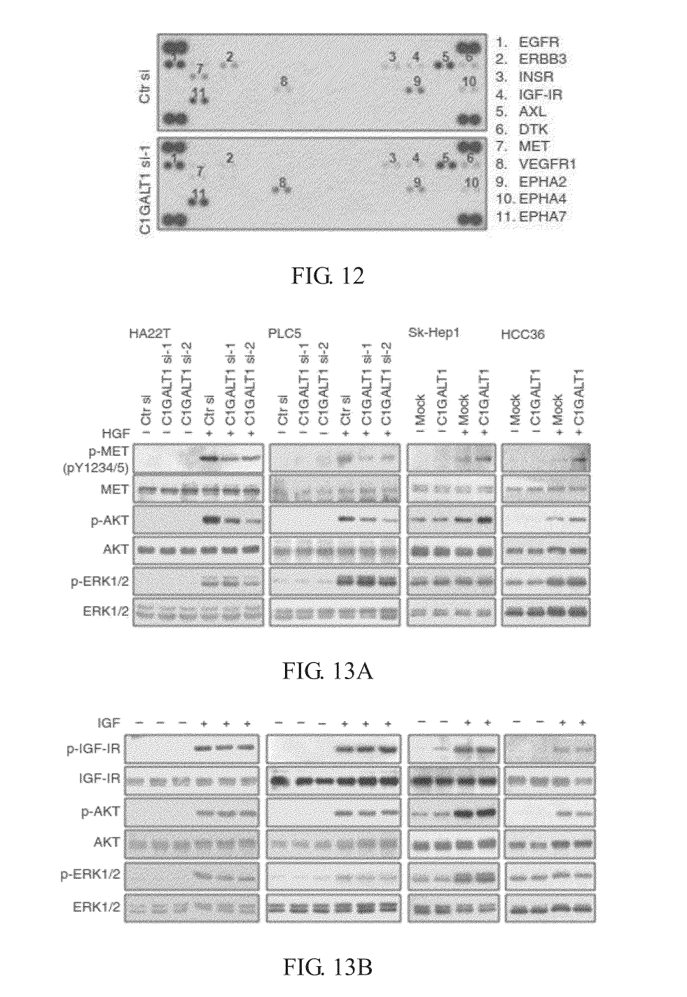

Because RTKs (receptor tyrosine kinase) are crucial for hepatocellular carcinoma proliferation and their activities have been found to be regulated by O-glycosylation, whether C1GALT1 could affect RTK signaling pathways in hepatocellular carcinoma cells were investigated. FIG. 12 is autoradiograms illustrating the effects of C1GALT1 on the phosphorylation of RTKs in hepatocellular carcinoma cells in accordance with an embodiment of the present invention. A human p-RTK array was used to detect the tyrosine phosphorylation level of 42 different RTKs. The cell lysates of control and C1GALT1 knockdown HA22T cells were applied to the p-RTK array. Our data indicated that knockdown of C1GALT1 in HA22T cells decreased phosphorylation of ERBB3, MET, and EPHA2, whereas phosphorylation of VEGFR1 was increased, as shown in FIG. 12. MET plays crucial roles in multiple functions in hepatocellular carcinoma, including cell proliferation, hepatocarcinogenesis, and metastasis. In addition, NetOGlyc 3.1 predicts one potential O-glycosylation site in the extracellular domain of MET (not shown).

FIGS. 13A-13B are autoradiograms illustrating the effects of C1GALT1 on the HGF-induced signaling and IGF-induced signaling in hepatocellular carcinoma cells in accordance with an embodiment of the present invention. The role of C1GALT1 in glycosylation and activation of MET in hepatocellular carcinoma cells was investigated. HA22T, PLC5, Sk-Hep1, and HCC36 cells were starved for 5 hours and then treated with (+)/without (−) HGF (25 ng/mL) or IGF (25 ng/mL) for 30 minutes. Cell lysates (20 mg) were analyzed by Western blotting with various antibodies, as indicated. Our results showed that knockdown of C1GALT1 (C1GALT1 si-1, C1GALT1 si-2) inhibited HGF-induced phosphorylation of MET at Y1234/5 and suppressed phosphorylation of AKT in HA22T and PLC5 cells, as shown in FIG. 13A. In contrast, overexpression of C1GALT1 enhanced HGF-induced activation of MET and increased p-AKT levels in Sk-Hep1 and HCC36 cells, as shown in FIG. 13B. In addition, C1GALT1 expression did not significantly alter IGF-I-induced signaling in all tested hepatocellular carcinoma cell lines, as shown in FIGS. 13C-13D. These results suggest that C1GALT1 selectively activates the HGF/MET signaling pathway.

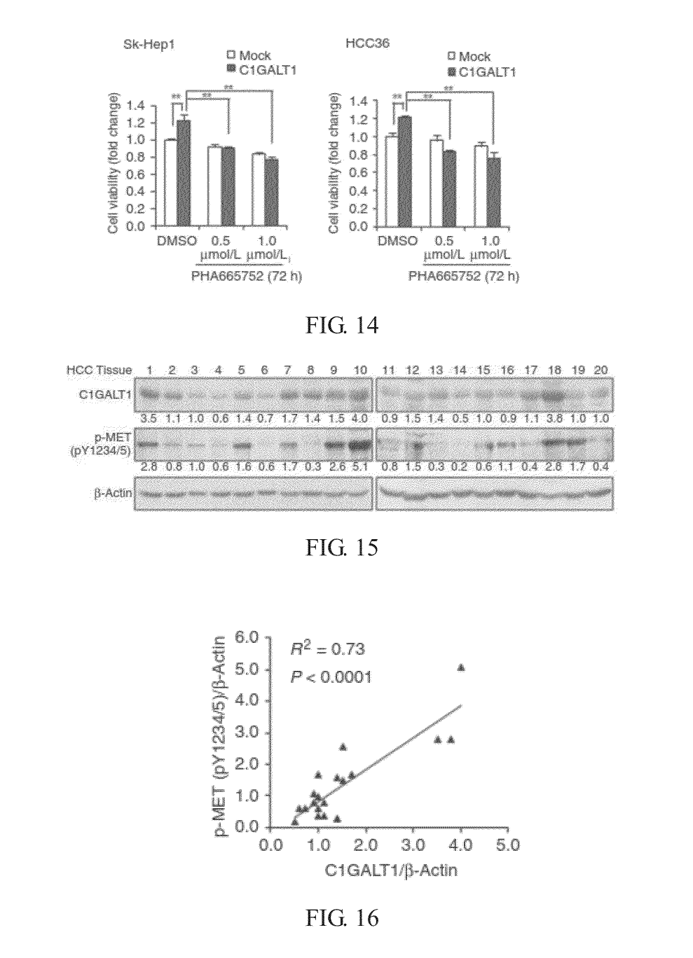

FIGS. 14A-14B are schematic diagrams illustrating the effects of MET inhibitor PHA665752 on C1GALT1-enhanced cell viability of hepatocellular carcinoma cells in accordance with an embodiment of the present invention. To investigate the role of the MET signaling pathway in C1GALT1-enhanced cell viability, Sk-Hep1 and HCC36 hepatocellular carcinoma cells were treated with PHA665757, a specific inhibitor of MET phosphorylation, at the indicated concentration and then analyzed by Trypan blue exclusion assays at 72 hours. Data are represented as means±SD from three independent experiments (** refers to P<0.01). Trypan blue exclusion assays showed that C1GALT1-enhanced cell viability was significantly inhibited by the blockade of MET activity, as shown in FIGS. 14A-14B. In addition, we observed that knockdown of C1GALT1 decreased HGF-induced cell migration and invasion, whereas overexpression of C1GALT1 enhanced HGF-induced cell migration and invasion (not shown).

FIG. 15 is an autoradiogram comparing C1GALT1 expression with MET phosphorylation in hepatocellular carcinoma tissues in accordance with an embodiment of the present invention. Tissue lysates (20 mg for each tumor) were analyzed by Western blotting. Signals of Western blotting were quantified by ImageQuant5.1. b-actin was a loading control. FIG. 16 is a graph illustrating the correlation of C1GALT1 expression and MET phosphorylation (p-MET) in 20 hepatocellular carcinoma tumors in accordance with an embodiment of the present invention. Pearson test was used to analyze the statistical correlation of C1GALT1 expression and p-MET in primary hepatocellular carcinoma tissues in FIG. 15. The results showed a significant correlation (R2=0.73, P<0.0001) between expression levels of C1GALT1 and p-MET. These results suggest that C1GALT1 could regulate MET activation in hepatocellular carcinoma.

### C1GALT1 Modifies O-Glycans on MET and Regulates HGF-Induced Dimerization of MET:

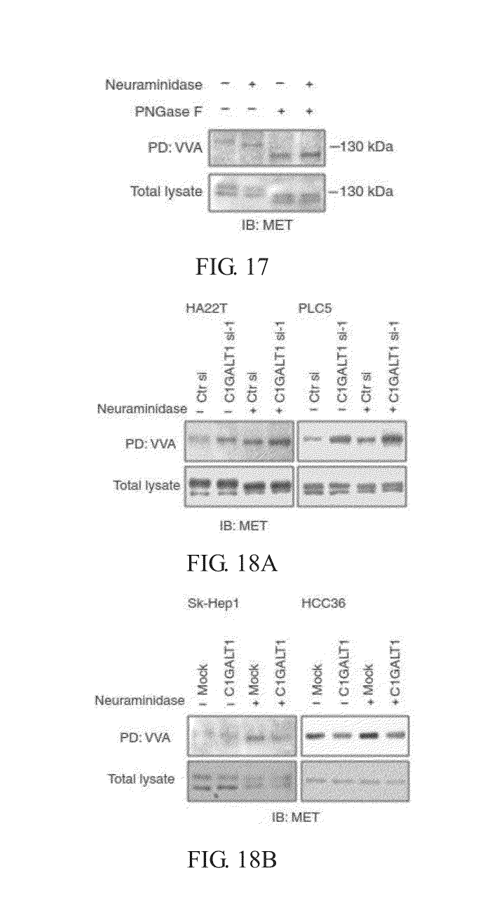

To investigate the mechanisms by which C1GALT1 regulates HGF/MET signaling, the effects of C1GALT1 on glycosylation and dimerization of MET in hepatocellular carcinoma cells was analyzed. Because C1GALT1 is an O-glycosyltransferase, whether MET is O-glycosylated was analyzed by using VVA and PNA lectins. VVA and PNA lectins recognize and bind to tumor-associated Tn and T antigen, respectively. FIG. 17 is an autoradiogram illustrating the N-glycan and the sialyl Tn antigen decoration on MET in accordance with an embodiment of the present invention. Lysates of HA22T cells were treated with neuraminidase for removing sialic acids and/or PNGaseF for removing N-glycan and then pulled down (PD) by VVA agarose beads. The pulled down proteins were analyzed by immunoblotting (IB) with anti-MET antibody. The molecular mass is shown on the right. Lectin pull-down assays with VVA or PNA agarose beads showed that endogenous MET expressed Tn and T antigens in all seven hepatocellular carcinoma cell lines tested (not shown). Moreover, VVA binding to MET in HA22T cells was further increased after removal of N-glycans on MET with PNGaseF, as shown in FIG. 17, indicating the specificity of VVA binding to O-glycans on MET. In addition, removal of sialic acids by neuraminidase enhanced VVA binding, suggesting that MET expresses sialyl Tn in addition to Tn, as shown in FIG. 17. These findings strongly suggest that MET expresses short mucin-type O-glycans in hepatocellular carcinoma cells.

To investigate whether C1GALT1 can modify O-glycans on MET, VVA binding to MET was analyzed in hepatocellular carcinoma cells with C1GALT1 knockdown or overexpression. FIGS. 18A-18B are autoradiograms illustrating the effects of C1GALT1 on O-glycosylation on MET in hepatocellular carcinoma cells in accordance with an embodiment of the present invention. Cell lysates were treated with (+) or without (−) neuraminidase and then pulled down by VVA agarose beads. The pulled down glycoproteins were immunoblotted (IB) with anti-MET antibody. Knockdown of C1GALT1 (C1GALT1 si-1) increased VVA binding to MET in both HA22T and PLC5 cells, as shown in FIG. 18A. Conversely, overexpression of C1GALT1 decreased VVA binding to MET in Sk-Hep1 and HCC36 cells, as shown in FIG. 18B. Consistently, removal of sialic acids enhanced VVA binding to MET in these cell lines, as shown in FIGS. 18A-18B. These findings indicate that C1GALT1 can modify O-glycans on MET in hepatocellular carcinoma cells. The effects of altered O-glycosylation on MET properties were investigated. The results showed that C1GALT1 expression did not significantly alter the protein level of MET analyzed by Western blotting, as shown in FIGS. 18A-18B and flow cytometry (data not shown).

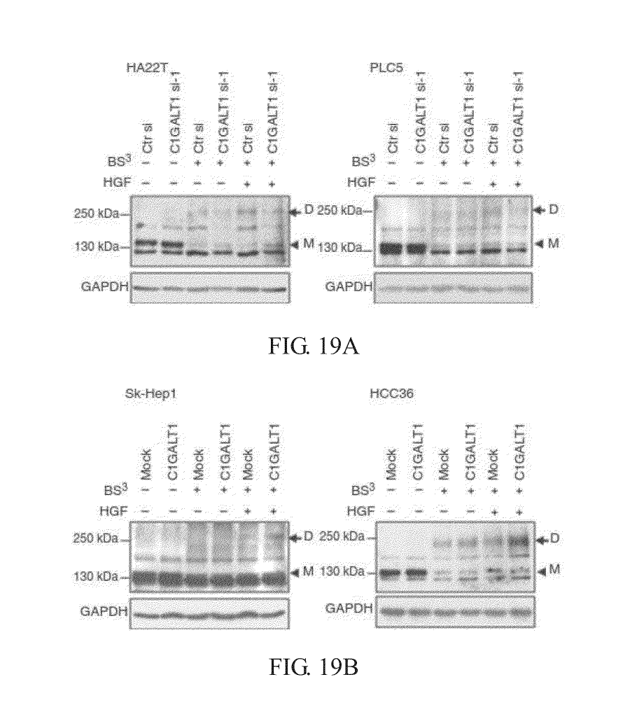

Because HGF-induced dimerization of MET is an initial and crucial step for the activation of MET signaling, whether C1GALT1 could affect MET dimerization was analyzed. FIGS. 19A-19B are autoradiograms illustrating the effects of C1GALT1 on dimerization of MET in hepatocellular carcinoma cells in accordance with an embodiment of the present invention. Hepatocellular carcinoma cells were starved for 5 hours and then treated with (+) or without (−) 25 ng/mL of HGF. Cell lysates were cross-linked by BS3 and then analyzed by Western blotting with anti-MET antibody. The arrows indicate the dimer (D) of MET, and the arrowheads indicate the monomer (M). Markers of molecular weight are shown on the left. GAPDH is an internal control. The data showed that knockdown of C1GALT1 (C1GALT1 si-1) suppressed HGF-induced dimerization of MET in both HA22T and PLC5 cells. In contrast, overexpression of C1GALT1 enhanced dimerization of MET in Sk-Hep1 and HCC36 cells as shown in FIGS. 19A-19B. These results suggest that C1GALT1 modifies O-glycans on MET and regulates HGF-induced dimerization of MET in hepatocellular carcinoma cells.

### Itraconazole Impairs C1GALT1 Function Leading to Accumulation of Tn Antigen Expression in Liver Cancer Cells:

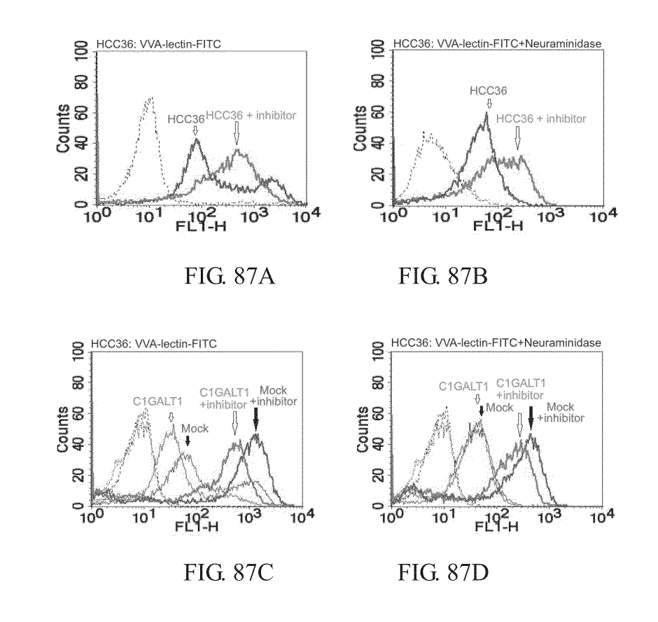

FIGS. 87A-87D are graphs illustrating that itraconazole impairs C1GALT1 function leading to accumulation of Tn antigen expression in lung cancer cells in accordance with an embodiment of the present invention. To confirm itraconazole binding to the catalytic domain of C1GALT1, the inventors conducted in vitro studies and revealed whether itraconazole impairs C1GALT1 function leading to Tn antigen accumulation. FIG. 87A shows HCC36 parental cells treated with (indicated by a long white arrow) or without (indicated by a short white arrow) itraconazole 10 ug/ml. After itraconazole treatment, cells are detached with 5 mM EDTA. Afterwards, FITC conjugated Vicia villosa agglutinin (VVA), lectin specifically recognizing Tn antigen, is applied for analysis with a fluoresencce-activated cell sorting (FACS) system. A right-shift of VVA peak indicates the accumulation of Tn antigens. FIG. 87B shows HCC36 parental cells treated with (indicated by a long white arrow) or without (indicated by a short white arrow) itraconazole 10 ug/ml. After itraconazole treatment, cells are detached with 5 mM EDTA and treated with or without 10 mU/ml neuraminidase to unmask the effects of sialylated O-glycans. After treatment with neuraminidase, FITC conjugated Vicia villosa agglutinin (VVA), lectin specifically recognizing Tn antigen, is applied for analysis with a fluoresencce-activated cell sorting (FACS) system. Our results show that itraconazole treatment leads to a right shift of the VVA peak regardless of the presence of sialylated O-glycans in the parental cells of HCC36 cell lines. To confirm whether the peak shift is a result of impaired C1GALT1 function, mock and C1GALT1 overexpressed HCC36 cells are analyzed. FIG. 87C shows mock (indicated by black arrows) and C1GALT1 (indicated by white arrows) overexpressed HCC36 cells with (indicated by long arrows) or without (indicated by long arrows) itraconazole 10 ug/ml treatment. Overexpression of C1GALT1 results in left-shift of the VVA peak in itraconazole-untreated transfectants, indicating intact C1GALT1 function in depleting Tn antigens as substrates for T antigen synthesis. Treatment of itracozole results in a major right-shift of the VVA peaks in both mock and C1GALT1 overexpressed transfectants indicating impaired C1GALT1 function leading to accumulation of Tn antigens. As shown in FIG. 87D, similar results are seen in mock and C1GALT1 overexpressed transfectants with sialylated O-glycans unmasked by neuraminidase. Overexpression of C1GALT1 in the cell line shows peak shifts to the left regardless of the presence of sialylated O-glycans compared with mock transfectants, indicating increased T antigen formation by depleting the available Tn antigen substrate. Treatment with itraconazole resulted in major peak shifts to the right in C1GALT1 overexpressed cells, denoting the accumulation of Tn antigen substrates and signifying impaired C1GALT1 function.

### Itraconazole Suppresses Liver Cancer Cell Growth:

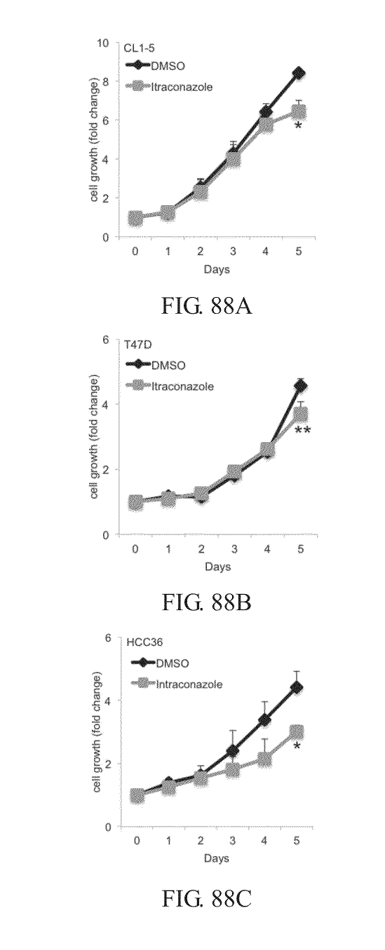

FIG. 88C is a schematic diagram illustrating that itraconazole suppresses liver cancer cell growth in accordance with an embodiment of the present invention. To determine whether impairing C1GALT1 function affects liver cancer cell malignant phenotypes, the effects of itraconazole on liver cancer cell growth are analyzed by MTT cell growth assay in HCC36 cell lines. Cells (3×10³) are seeded into 96-well microtitre plates and treated with DMSO (control) or itraconazole 10 g/ml for 5 days at 37° C. under 5% CO₂ atmosphere. Results from two independent experiments are presented as mean±SD and analyzed with student's t-test, *p<0.05 and **p<0.01. FIG. 88C shows that liver cancer cell growth is significantly suppressed by itraconazole compared with control (DMSO).

In summary, the results described above show that overexpression of C1GALT1 in hepatocellular carcinoma tissues is associated with advanced tumor stage, metastasis, as shown in Table 1 and poor prognosis, as shown in FIG. 5. C1GALT1 expression regulated hepatocellular carcinoma cell viability and proliferation in vitro and in vivo, as shown in FIGS. 9-11. The C1GALT1-enhanced cell viability is inhibited by MET inhibitor, as shown in FIGS. 14A-14B. MET carried O-glycans, and these structures are modified by C1GALT1. Furthermore, C1GALT1 could regulate HGF-induced dimerization and activity of MET in hepatocellular carcinoma cells, as shown in FIGS. 19A-19B. Taken together, the data are the first to show that C1GALT1 is able to regulate hepatocellular carcinoma cell proliferation in vitro and in vivo, and modulation of O-glycosylation and activity of MET may be involved in this process.

MET is also called hepatocyte growth factor receptor (HGFR). The MET gene is an important protooncogene in a variety of human cancers. The data showed that MET from all tested seven hepatocellular carcinoma cell lines could be pulled down by VVA and PNA lectins, suggesting mucin-type O-glycans (or Tn and T antigens) decorated on MET. C1GALT1 modulates the expression of mucin-type O-glycans on MET, as shown in FIGS. 18A-18B. Removal of sialic acids by neuraminidase enhanced VVA binding to MET, indicating that some of the Tns are sialylated in hepatocellular carcinoma cells. In cancer, an increase in the expression of short O-glycans, such as Tn, sialyl Tn, T, and sialyl T, often alters the function of glycoproteins and their antigenic property, as well as the potential of cancer cells to invade and metastasize. Removal of N-glycans by PNGaseF further enhanced VVA binding to MET. These results strongly suggest the presence of O-glycans on MET.

Glycosylation has long been proposed to control various protein properties, including dimerization, enzymatic activity, secretion, subcellular distribution, and stability of RTKs. However, most studies focused on effects of N-glycans on RTKs Importantly, C1GALT1 can modulate the O-glycans on MET and enhance dimerization of MET, as shown in FIGS. 19A-19B and phosphorylation of MET, as shown in FIGS. 15-16. Because receptor dimerization is a key regulatory step in RTK signaling, it is highly possible that C1GALT1 modulates MET activity via the enhancement of its dimerization. MET expresses O-glycans and changes in these carbohydrates regulate the activity of MET. Aberrant activation of MET signaling correlates with the increased cell proliferation, poor prognosis, and poor outcome of human hepatocellular carcinoma. HGF/MET signaling has been shown to promote invasion and metastasis of hepatocellular carcinoma cells. The data show that C1GALT1 can increase dimerization and phosphorylation of MET, which is consistent with previous findings that C1GALT1 can enhance HGF-induced migration and invasion. Targeting MET is also an attractive strategy for treating many human cancers, including hepatocellular carcinoma.

C1GALT1 expression modulates HGF-, but not IGF-mediated signaling, suggesting the selectivity of C1GALT1 activity toward certain RTKs, as shown in FIGS. 13A-13B. However, that C1GALT1 expression changes binding patterns of VVA to several glycoproteins was observed (not shown). Knockdown of C1GALT1 in hepatocellular carcinoma cells also modulates phosphorylation of, VEGFR1, and EPHA2, as shown in FIG. 12, suggesting that there are other acceptor substrates, in addition to MET. Therefore, it remains possible that several signaling pathways may be involved in mediating the biologic functions of C1GALT1 in hepatocellular carcinoma cells. Thus, targeting C1GALT1 could have effects similar to those from targeting multiple RTKs. The present invention opens up avenues for treating cancers by targeting not only the receptors themselves but also their O-glycosylation regulators.

### Colorectal Cancer

Colorectal carcinoma (CRC) is one of the major causes of cancer related morbidity and mortality. In the western countries, it is the second leading cause of cancer death. In the United States, there are approximately 140,000 new cases and 50,000 deaths per year. Although the introduction of new surgical concepts, emergence of new biologic agents, availability of several new chemotherapeutic drugs, and use of various combinations thereof have dramatically improve the survival of patients with stage IV colorectal cancer, generally speaking, colorectal cancers that spread to distant organs are usually not curable. This problem raises questions as to whether current systemic anti-colorectal cancer treatments are targeting molecular mechanisms that are truly critical to cancer growth and metastasis. The methods in accordance with hepatocellular carcinoma embodiments of the present invention are described as follows. However, the following materials and specific procedures provided are illustrative only and not are intended to be limiting.

### Colorectal Cancer Tissue Resource and Cell Culture Conditions

Post surgery human colorectal cancer tissues are collected for RNA extraction, Western blotting and paraffin-embedded tissue sections. The use of human tissues for this study is approved by the local hospital ethics committee, and written consent is obtained from each patient before collecting samples.

Human colon cancer cell lines HCT116, SW480, SW620, Caco2, HT29, Colo205 are purchased from Bioresource Collection and Research Center (Hsinchu, Taiwan). Cells are maintained with Dulbecco's modified Eagle's medium (Thermo scientific, Barrington, Ill.) containing 10% fetal bovine serum (GIBCO, Grand Island, N.Y.), 100 IU/mL penicillin, and 100 mg/mL streptomycin in a humidified tissue culture incubator at 37° C., 5% CO2.

### Methods of Inhibition of C1GALT1 Expression and Activity in Colon Cancer Cells

According to an embodiment of the present invention, in the experiment in vitro, the pLKO/C1GALT1-shRNA plasmid containing the sequence, 5′-CCCAGCCTAATGTTCTTC ATA-3′(SEQ ID NO: 13), against C1GALT1 is used as the C1GALT1 inhibition substance. The pLKO/C1GALT1-shRNA and non-targeting pLKO plasmids (as a negative control) are purchased from National RNAi Core Facility (Academia Sinica, Taipei, Taiwan). For C1GALT1 knockdown, HCT116 and SW620 cells are transfected with 100 nM of shRNA and selected with 2 ug/mL of puromycin for 14 days.

According to another embodiment of the present invention, in the experiment in vivo, the pLKO/C1GALT1-shRNA plasmid containing the sequence, 5′-CCCAGCCTAATG TTCTTCATA-3′(SEQ ID NO: 13), against C1GALT1 is used as the C1GALT1 inhibition substance. The pLKO/C1GALT1-shRNA and non-targeting pLKO plasmids (as a negative control) are purchased from National RNAi Core Facility (Academia Sinica, Taipei, Taiwan). For C1GALT1 knockdown, HCT116 and SW620 cells are transfected with 100 nM of shRNA and selected with 2 ug/mL of puromycin for 14 days. Knockdown of C1GALT1 in single colonies is confirmed by Western blotting.

For in vivo metastasis analysis, stable cell lines (2×10⁶ cells in 100 n1 PBS) containing the pLKO/C1GALT1-shRNA plasmid and the non-targeting pLKO plasmid are submucosally injected into the rectum of 6-week-old female NOD-SCID mice (National Laboratory Animal Center, Taiwan) at day 0. The health status is monitored. After 6 weeks, mice are euthanized and inspected for any tumors formed Animal experiments are reviewed and approved by the Institutional Animal Care and Use Committee (IACUC) of National Taiwan University College of Medicine.

For in vivo tumor growth analysis, 6-week-old female NOD-SCID mice (National Laboratory Animal Center, Taiwan) are injected subcutaneously with 1×10⁷ of cells containing the pLKO/C1GALT1-shRNA plasmid and the non-targeting pLKO plasmid. Tumors are allowed to develop for 6 weeks and measured with calipers every 3 days. The volume of tumors is estimated based on the formula α²×β/2, where a is the smaller diameter and β is the larger diameter. At day 42 after injection, tumors in each group are excised for analyses Animal experiments are reviewed and approved by the Institutional Animal Care and Use Committee (IACUC) of National Taiwan University College of Medicine.

### Methods for Overexpressing C1GALT1 in Colon Cancer Cells

For C1GALT1 overexpression, HCT116 and SW480 cells are transfected with pcDNA3.1/C1GALT1/mycHis plasmids, which contains a promoter and cDNA of C1GALT1, shown as SEQ ID NO: 4 in the sequence list, by using Lipofectamine® 2000 (Invitrogen, Life Technologies Inc., Grand Island, N.Y.) according to the manufacturer's protocols. Empty pcDNA3.1/mycHis plasmids are used as mock transfection. Stable clones are further selected with 400 ug/mL of G418 for 14 days.

### Analysis Procedures of Effects of C1GALT1 Inhibition on Colorectal Cancer

### Tissue Array and Immunohistochemistry:

Paraffin-embedded tissue sections are incubated with an anti-C1GALT1 (1:300) antibody overnight at 4° C. Super Sensitive link-Label IHC detection System (BioGenex, California, CA) is used and signals are visualized with 3,3-diaminobenzidine (DAB) liquid substrate system (Sigma, St. Louis, Mo.). All sections are counterstained with hematoxylin. Negative controls are performed by replacing primary antibody with a control IgG at the same concentration. The staining intensity and positive ratio of C1GALT1 are observed under microscopy by two independent scorers blinded to the clinical parameters.

### Western Blotting:

Equal amounts of protein samples are mixed with 5× sample buffer and boiled for 5 minutes, separated on SDS-polyacrylamide gels, and then transferred to PVDF membrane. The membranes are blocked in 5% BSA for 1 hour at room temperature, and incubated with primary antibodies overnight at 4° C. Anti-phosphotyrosine (4G10) antibody (Millipore, Billerica, Mass.), antibodies against C1GALT1, GAPDH, FGFR2 (Santa Cruz Biotechnology, Santa Cruz, Calif.) and anti-β-actin antibody (BD Pharmingen, San Jose, Calif.) are used. The blots are then incubated with secondary antibody conjugated with horseradish peroxidase and immunoreacted bands are detected by ECL reagents and exposed on x-ray film. cDNA synthesis and quantitative real-time RT-PCR:

The total RNA is extracted by Trizol reagent (Invitrogen) according to the manufacturer's protocol. Extracted RNA is then reverse transcribed using the Superscript III First-Strand cDNA Synthesis Kit (Invitrogen). Quantitative PCR System Mx3000P (Stratagene, La Jolla, Calif.) is used for quantitative real-time RT-PCR. Reactions are performed in a 20 μl volume with 2 μl cDNA, 400 nM of sense and anti-sense primers, and 10 μl Brilliant®SYBR®Green Q-PCR Master Mix (Stratagene). Primer sets are designed as the following:

NANOG,
sense
(SEQ ID NO: 9)
(5′-GGCCTCAGCACCTACCTACCC-3′)
and
anti-sense
(SEQ ID NO: 10)
(5′-TCCAAGGCAGCCTCCAAGTCA-3′);
OCT4,
sense
(SEQ ID NO: 11)
(5′-GCAGATCAGCCACATCGCCC-3′)
and
anti-sense
(SEQ ID NO: 12)
(5′-GCCCAGAGTGGTGACGGAGA-3′)
GAPDH,
sense
(SEQ ID NO: 7)
(5′-ACAGTCAGCCGCATCTTCTT-3′)
and
anti-sense
(SEQ ID NO: 8)
(5′-GACAAGCTTCCCGTTCTCAG-3′)

### Relative Quantity of Gene Expression Normalized to GAPDH is Analyzed Using MxPro Software (Stratagene).

### Cell Growth Assay:

Cells (2.0×103) are seeded in 96-well plates. After culture for different time periods, the 3-(4,5 dimethyl-2 thiazolyl)-2,5 diphenyl-2H tetrazolium bromide solution (MTT; Sigma-Aldrich) is added to a final concentration of 0.5 mg/mL and incubated for 4 hours at 37 to allow MTT reduction. The formazan crystals are then dissolved in 10% sodium dodecyl sulfate (SDS) containing 0.01 M HCl and absorbance is measured at the dual wavelengths of 570 and 630 nm with a spectrophotometer.

### Transwell Migration Assay:

Cells (8×10⁴) in 500 μL DMEM containing 1% FBS are seeded into the upper part of the Boyden chamber with 8-μm pore filters (Corning, Cambridge, Mass.). Cell migration is induced by 10% FBS (GIBCO) or 25 ng/mL bFGF (Sigma) in the lower part. After 48 hours, cells that had migrated to the lower surface of the membrane are fixed and stained with 0.5% crystal violet (Sigma) and counted under a microscope in six random fields.

### Matrigel Invasion Assay

Cell invasion assays are performed in BioCoat Matrigel Invasion Chambers (Becton-Dickinson, Bedford, Mass.) according to the manufacturer's protocol. Briefly, 500 μL DMEM containing 10% FBS (GIBCO) or 25 ng/mL bFGF (Sigma) are added to the lower part of the chamber, whereas cells (8×10⁴) in 500 μL DMEM are seeded to the upper part. Cells are allowed to invade the matrigel for 48 hours. Cells that invaded to the lower surface of the membrane are fixed and stained with 0.5% crystal violet (Sigma) and counted under a microscope in six random fields.

### Sphere Formation Assay

Cells are suspended in DMEM/F12 (1:1) supplemented with B27 supplement (Invitrigen), epidermal growth factor and recombinant fibroblast growth factor basic (Sigma). EGF (20 ng/mL) and bFGF (25 ng/mL) are used together or separately depending on experimental design. The cells are seeded in Ultra-Low attachment 24-well plates (Corning) at a density of 1,000 cells per well. After 10-14 days, MTT solution (Sigma) are added to visualize any sphere formed and pictures are taken. The number of spheres is quantified by ImageJ 1.42q software (Wayne Rasband).

### Lectin Pull Down and Immunoprecipitation

To detect the T, Tn, sialyl T and sialyl Tn antigens on FGFR2, peanut agglutinin (PNA) and Vicia villosa agglutinin (VVA) lectins conjugated agarose beads (Vector Laboratories, Burlingame, Calif.) are used; neuraminidase is used to remove sialyl acid. Briefly, 1-2 mg of cell lysates are treated with or without neuraminidase at 37° C. for 1 h and incubated with PNA or VVA overnight at 4° C. For immunoprecipitation, cell lysates are incubated with 2.5 μg FGFR2 antibody overnight at 4° C. Next, protein G sepharose beads (GE Healthcare Life Sciences, Piscataway, N.J.) are added and incubated at 4° C. for 4 hours. The precipitated agarose beads are washed several times with lysis buffer to remove any unbound protein and then subjected to western blotting.

### Statistical Analysis:

Quantitative data from at least three independent experiments are expressed as means (±SD). Student's t-tests are used to compare the differences between groups. Kaplan-Meier analysis and the log-rank test are used to estimate overall survival. A P of <0.05 is considered statistically significant.

### Analysis Results of Effects of C1GALT1 Inhibition on Colorectal Cancer

C1GALT1 Expression is Up-Regulated in CRC and is Associated with Poor Survival:

FIG. 20 is an autoradiograms illustrating C1GALT1 expression in colorectal cancer tissues (T) and non-tumor tissues (N) analyzed by Western blotting in accordance with an embodiment of the present invention. To determine the expression levels of C1GALT1 in human CRC, paired CRC (n=8) from patients of the National University Hospital (NTUH) were use and analyzed by Western blot, as shown in FIG. 20. C1GALT1 expression is found to be up-regulated in CRC tumors compared with adjacent non-tumor tissues.

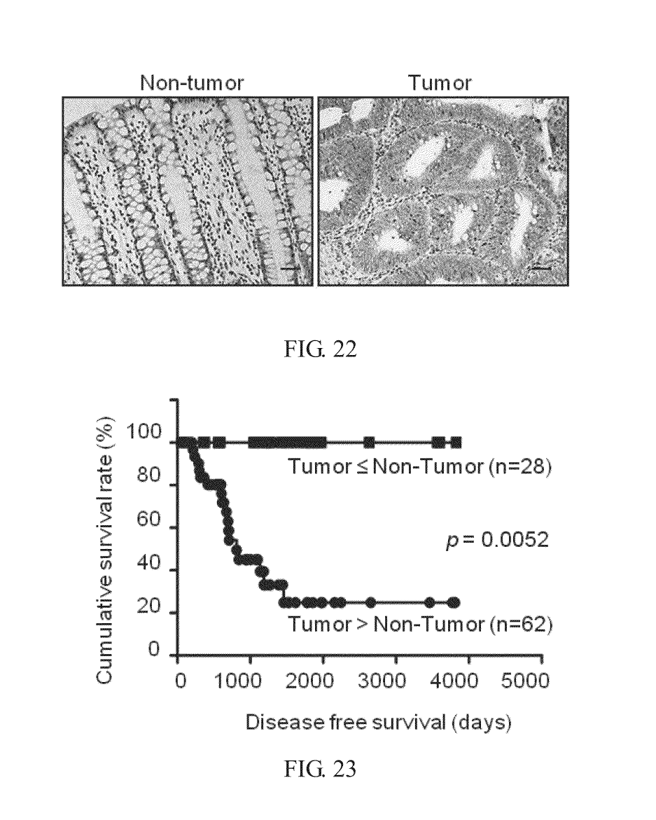

FIG. 21 is an image illustrating C1GALT1 expression in colorectal cancer tissues and non-tumor tissues analyzed by immunohistochemistry in accordance with an embodiment of the present invention. FIG. 22 is a schematic diagram illustrating the comparison of the C1GALT1 immunohistochemistry staining results between colorectal cancer tissues (T) and non-tumor tissues (N) in accordance with an embodiment of the present invention. The scale bars are 50 μm. To confirm this finding, C1GALT1 immunohistochemistry (IHC) staining was performed, as shown in FIG. 21 in CRC patients (n=90) from NTUH. Results show that 68.9% (62/90) of paired CRC tissues expressed increased levels of C1GALT1 compared with adjacent non-tumor parts, as shown in FIG. 22.

FIG. 23 is a graph illustrating the correlation between survival rates and the immunohistochemistry staining results in colorectal cancer in accordance with an embodiment of the present invention. Kaplan-Meier survival analysis of CRC tissues (n=90) shows that, compared with adjacent non-tumor tissues, CRC patients with elevated C1GALT1 expression levels in tumors (T>N) are associated with significantly lower survival rate while patients with tumors expressing lower C1GALT1 levels (T<N) are associated with better survival as shown in FIG. 23. These findings suggest that C1GALT1 expression is frequently up-regulated in CRC and its overexpression is associated with poor survival. C1GALT1 is differentially expressed in colon cancer cell lines:

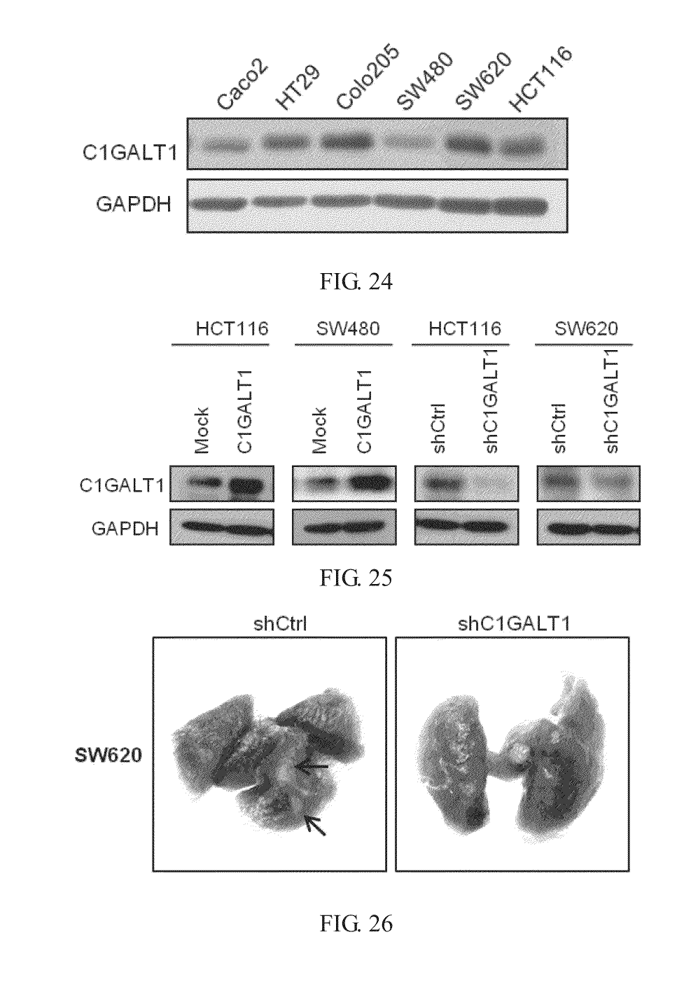

To understand the role of C1GLAT1 in CRC, C1GALT1 expression was analyzed in six colon cancer cell lines: Caco2, HT29, Colo205, SW480, SW620, and HCT116 by Western blot. FIG. 24 is an autoradiogram the expression of C1GALT1 in 7 colorectal cancer cell lines analyzed by Western blotting in accordance with an embodiment of the present invention. The results show that C1GALT1 expression level is higher in SW620 than in SW480, which correlates with their clinical malignant phenotypes corresponding to metastatic and non-metastatic origin respectively where both cell lines were isolated from the same patient. Thus HCT116 and SW480 were selected for C1GALT1 overexpression with C1GALT1/pcDNA3.1 while HCT116 and SW620 were selected for C1GALT1 knockdown with C1GALT1 specific shRNA.

FIG. 25 is an autoradiogram illustrating the overexpression and knockdown of C1GALT1 in colon cancer cells confirmed by Western blotting in accordance with an embodiment of the present invention. Overexpression of C1GALT1 in HCT116 and SW480 was achieved by transfection with vector (Mock) or C1GALT1/pcDNA3.1 plasmid (C1GALT1) and maintained with G418. Knockdown of C1GALT1 in HCT116 and SW620 was achieved by transfection with control (shCtrl) or C1GALT1 shRNA (shC1GALT1) and maintained with puromycin.

### C1GALT1 Regulates Malignant Phenotypes of Colon Cancer Cell Lines In Vivo:

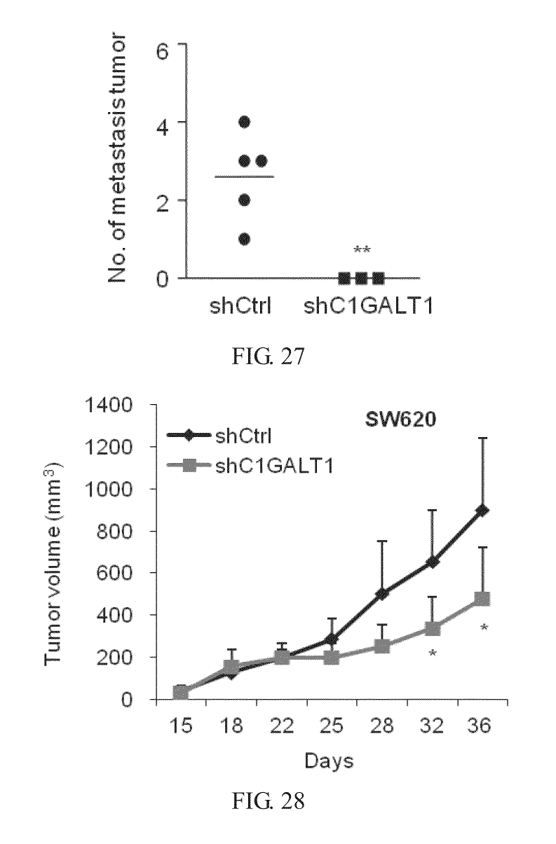

To investigate the effect of C1GALT1 on tumor metastasis in vivo, the rectal xenograft model was performed by submucosally injecting control and C1GALT1 knockdown SW620 cells into the rectum of NOD-SCID mice. FIG. 26 is an image illustrating metastasis tumor nodules in the lungs in accordance with an embodiment of the present invention. Arrows indicate metastatic tumor nodules. FIG. 27 is a schematic diagram illustrating the total number of metastasis tumor nodules in accordance with an embodiment of the present invention. That C1GALT1 knockdown (shC1GALT1) suppressed lung metastasis was observed, as shown in FIG. 26. The total number of metastatic tumors from all organs and abdominal wall were counted, as shown in FIG. 27.

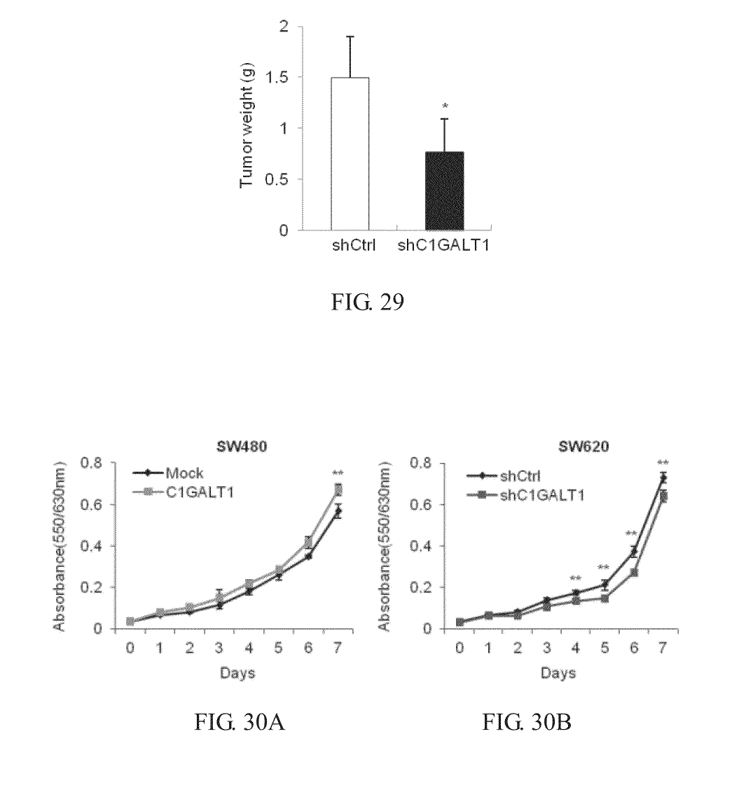

FIG. 28 is a schematic diagram illustrating the tumor number of colorectal cancer in a control group (shCtrl) and a C1GALT1 knockdown group (shC1GALT1) relative to days in accordance with an embodiment of the present invention. FIG. 29 is a schematic diagram illustrating the tumor weight of colorectal cancer in a control group (shCtrl) and a C1GALT1 knockdown group (shC1GALT1) relative to days in accordance with an embodiment of the present invention. Tumor growth model in vivo was performed by subcutaneous injection of control and C1GALT1 knocked SW620 cells (1×10⁷, n=6 for each group). Tumor growth was measured every 3 days, and tumors were excised after 6 weeks. Data are presented as mean±SD. *p<0.05; **p<0.01. The tumor volume and weight were significantly smaller in mice injected with C1GALT1 knocked down cells, as shown in FIGS. 28-29. These results suggest that C1GALT1 knockdown suppresses tumor metastasis and growth in vivo. C1GALT1 regulates malignant phenotypes and stem-like properties of colon cancer cell lines:

To analyze CRC malignant phenotypes, cell viability, migration and invasion assays were performed. FIG. 30 is schematic diagrams illustrating the effect of C1GALT1 on cell growth of colorectal cancer cells in accordance with an embodiment of the present invention. C1GALT1 overexpression in SW480 (C1GALT1) enhanced (left panel) while knockdown in SW620 (shC1GALT1) suppressed cell growth analyzed by MTT assays for 7 days. **p<0.01. Results from MTT assay show that overexpression (C1GALT1) of C1GALT1 in SW480 increased while knockdown of C1GALT1 (shC1GALT1) in SW620 inhibited cell viability, as shown in FIG. 30.

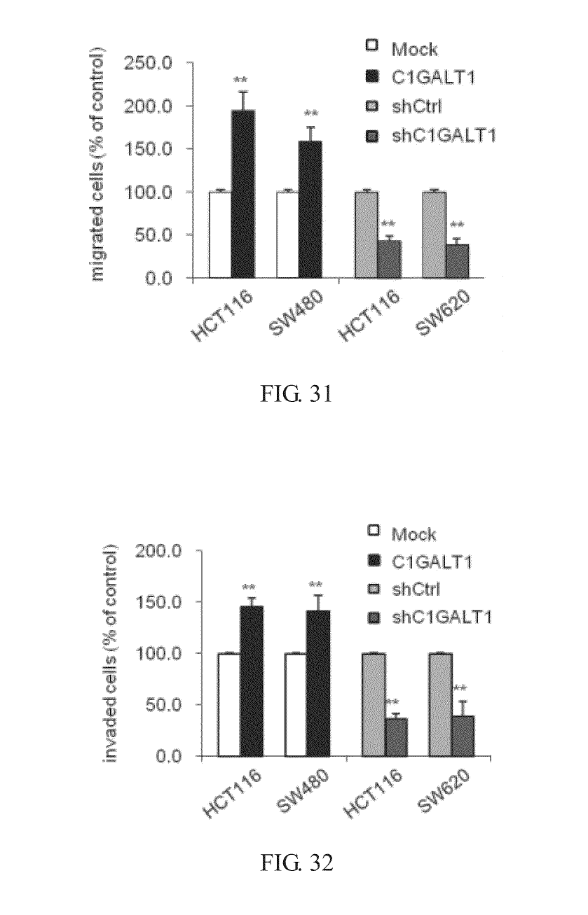

FIG. 31 is a schematic diagram illustrating the effect of C1GALT1 on migration of colorectal cancer cells in accordance with an embodiment of the present invention. FIG. 32 is a schematic diagram illustrating the effect of C1GALT1 on the invasion of colorectal cancer cells in accordance with an embodiment of the present invention. C1GALT1 overexpression (C1GALT1) enhanced while knockdown (shC1GALT1) suppressed CRC cell migration. DMEM containing 10% FBS were used as chemoattractants. After 48 hours the number of migrated cells from 6 random fields was counted. Results obtained are displayed as percentage of migrated or invaded cells relative to control (shCtrl, Mock) and student's t-test expressing as mean±SD, **p<0.05. C1GALT1 overexpression enhanced while knockdown (shC1GALT1) suppressed CRC cell invasion analyzed by Matrigel™ coated transwell systems. Overexpression of C1GALT1 (C1GALT1) significantly enhanced cell migration and invasion in colon cancer cells, in contrast, knockdown of C1GALT1 (shC1GALT1) suppressed cell migration and invasion, as shown in FIGS. 31-32.

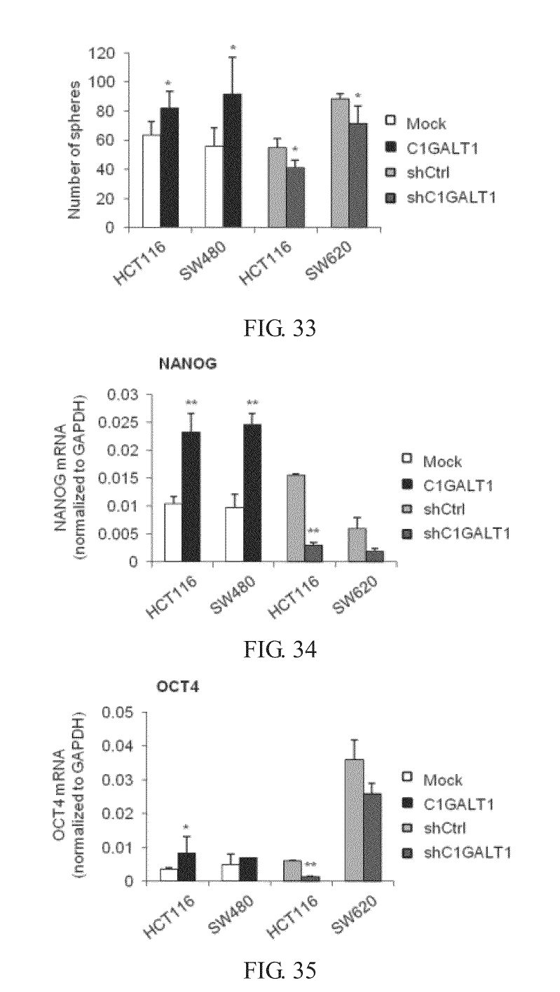

Since the cancer stem-like cell property is very critical for cancer malignancy and is gaining increasing attention, whether C1GALT1 is able to modulate the stem-like properties in colon cancer cell lines was investigated. FIG. 33 is a schematic diagram illustrating the effects of C1GALT1 on sphere formation of colorectal cancer cells in accordance with an embodiment of the present invention. Sphere formation assays were performed to analyze the self-renewal ability. The number of spheres formed was counted after visualizing by MTT. Results are presented as mean±SD from three independent experiments. *p<0.05; **p<0.01. As shown in FIG. 33, overexpression of C1GALT1 formed significantly more spheres compared with mock transfectants. On the other hand, knockdown of C1GALT1 (shC1GALT1) inhibited the number of spheres formed.

Apart from sphere formation, the stem cell associated markers, NANOG and OCT4, was also analyzed by quantitative real-time RT-PCR. FIGS. 34-35 are schematic diagrams illustrating the effects of C1GALT1 on stem cell-like properties of colorectal cancer cells in accordance with an embodiment of the present invention. Both NANOG and OCT4 mRNA levels were up-regulated in C1GALT1 overexpressing cells (C1GALT1) and down-regulated in C1GALT1 knocked down cells (shC1GALT1), as shown in FIGS. 34-35, *p<0.05; **p<0.01. These findings suggest that C1GALT1 enhances malignant behaviors and stem-like properties of colon cancer cell lines in vitro.

C1GALT1 Regulates bFGF-Induced Malignant Phenotypes and Stem-Like Properties in Colon Cancer Cells:

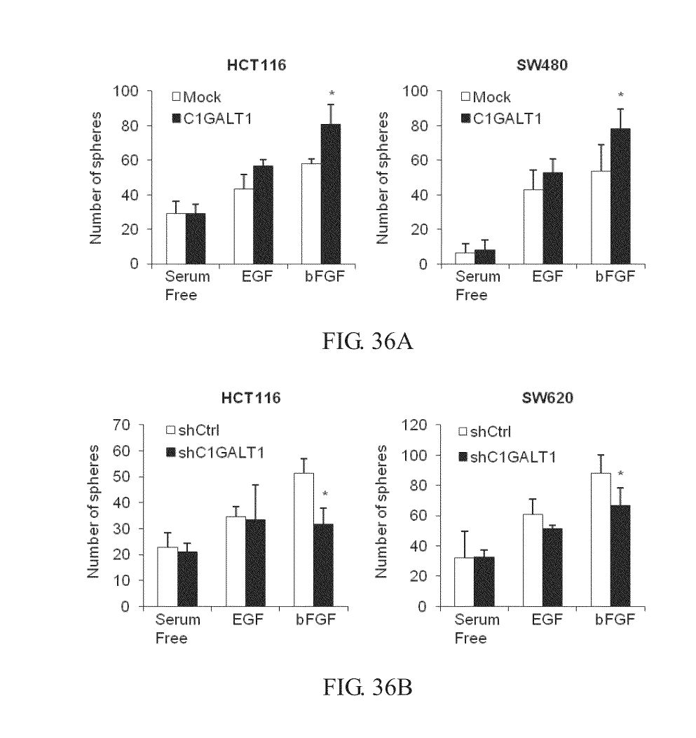

In order to further explore the effect of C1GALT1 on colon cancer, which signaling pathway C1GALT1 regulated colon cancer cell malignant phenotypes was investigated. As EGF and bFGF play critical roles in malignant progression and stemness in many cancers and both were needed in performing sphere formation assays, we therefore used these two growth factors separately to examine their effects. FIGS. 36A-36B are autoradiograms illustrating the effects of C1GALT1 on the bFGF-induced signaling and EGF-induced signaling in sphere formation of colorectal cancer cells in accordance with an embodiment of the present invention. Sphere formation assay were performed on cells in serum free, EGF (epidermal growth factor, 20 μg/ml) or bFGF (basic fibroblast growth factor, 25 μg/ml) treatments supplemented with 1×B27. The number of spheres formed was counted. Results are presented as mean±SD from three independent experiments. That overexpression of C1GALT1 (C1GALT1) enhanced bFGF-induced sphere formation is found, as shown in FIG. 36A. Knockdown of C1GALT1 (shC1GALT1) showed suppressed bFGF-induced sphere formation, as shown in FIG. 36B. In contrast, there are no significant differences in EGF-induced sphere formation.

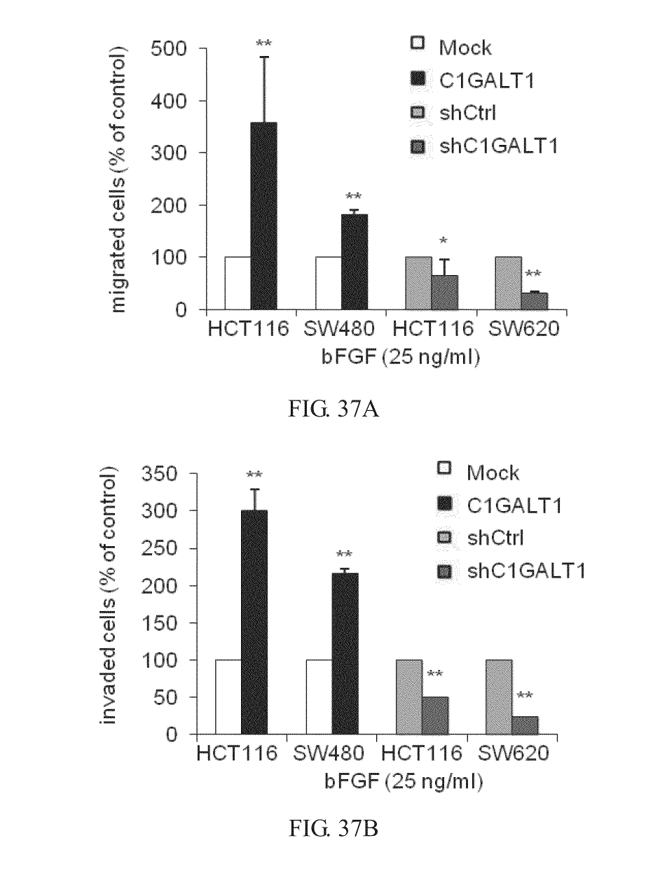

FIGS. 37A-37B are autoradiograms illustrating the effects of C1GALT1 on the bFGF-induced signaling in migration and invasion of colorectal cancer cells in accordance with an embodiment of the present invention. Serum free DMEM containing bFGF (25 ng/ml) were used as chemoattractants. Results obtained are displayed as percentage of migrated or invaded cells relative to control and student's t-test expressing as mean±SD. C1GALT1 overexpression (C1GALT1) enhanced while knockdown (shC1GALT1) suppressed bFGF-induced CRC cell invasion analyzed by Matrigel™ coated transwell systems. * p<0.05; ** p<0.01. Overexpression of C1GALT1 (C1GALT1) promoted bFGF-induced migration and invasion, whereas knockdown of C1GALT1 (shC1GALT1) blocked bFGF-induced cell migration and invasion, as shown in FIGS. 37A-37B. Collectively, these results indicate C1GALT1 regulated cell migration, invasion and stem-like properties through bFGF-mediated pathways.

C1GALT1 Regulates O-Glycosylation and bFGF-Induced FGFR2 Activation in Colon Cancer Cell Lines:

As C1GALT1 was found to regulate bFGF-induced malignant phenotypes, whether FGFR glycosylation and activity were modified by C1GALT1 expression was investigated. Among the four FGFRs (FGFR1-4), FGFR2 in particular has recently been reported to be critically involved in colorectal cancer. Since C1GALT1 is an extension enzyme of the GalNAc-O-glycosylation, whether O-glycans are decorated on FGFR2 in colon cancer cell lines was first investigated.

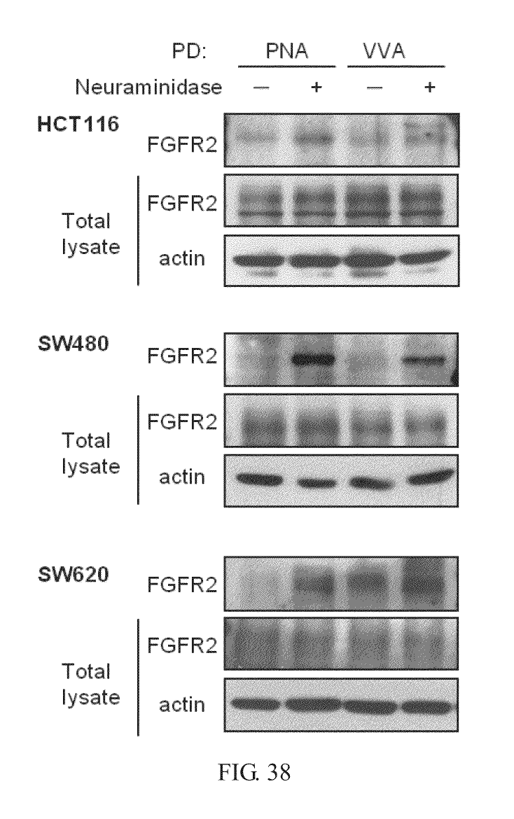

FIG. 38 is an autoradiogram illustrating the sialyl T and the sialyl Tn antigen decoration on FGFR2 in colorectal cancer cells in accordance with an embodiment of the present invention. Total cell lysates of HCT116, SW480 and SW620 were treated with (+) or without (−) neuraminidase for removing sialic acids, and T and Tn antigens were respectively pulled down by either PNA or VVA lectin agarose beads and blotted with anti-FGFR2 antibody, indicating that sialyl T and sialyl Tn were also decorated on FGFR2, as shown in FIG. 38.

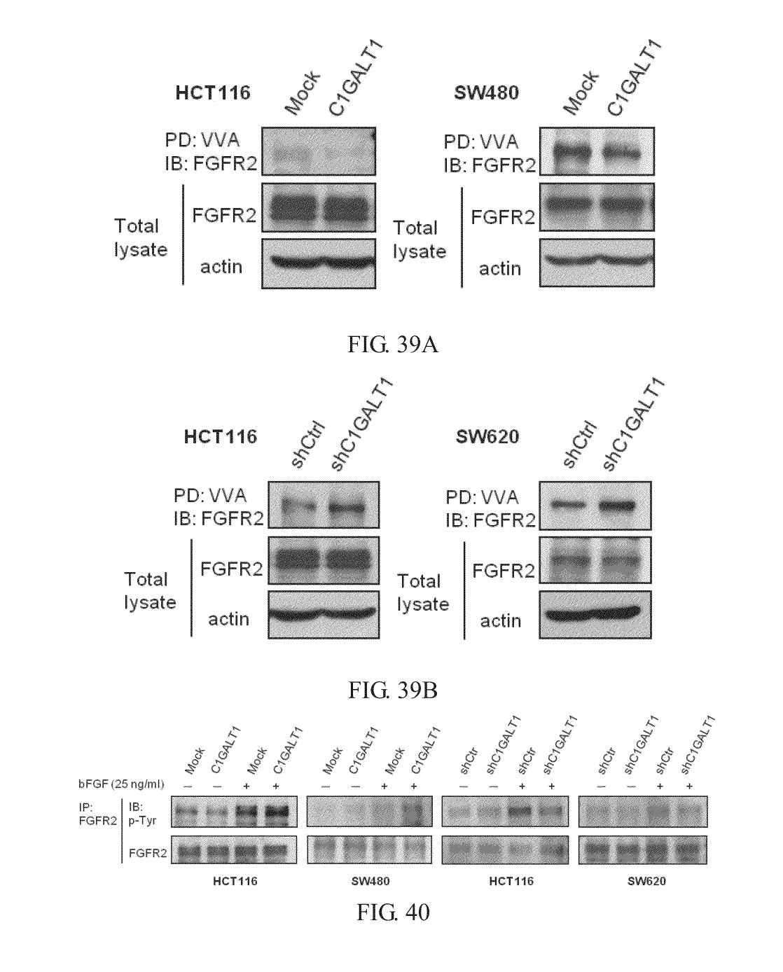

FIGS. 39A-39B are autoradiograms illustrating the effects of C1GALT1 on the O-glycosylation of RGFR2 in colorectal cancer cells in accordance with an embodiment of the present invention. To investigate whether C1GALT1 can modify O-glycans on FGFR2, total cell lysates from C1GALT1 overexpressing HCT116 and SW480 cells and C1GALT1 knocked down HCT116 and SW620 cells were subjected to VVA lectin pull down and immunoblotted with anti-FGFR2 antibody. Actin was used as internal control. Overexpression of C1GALT1 (C1GALT1) decreased VVA binding to FGFR2, as shown in FIG. 39A, whereas knockdown of C1GALT1 (shC1GALT1) increased VVA binding to FGFR2, as shown in FIG. 39B. Since C1GALT1 can alter O-glycosylation of FGFR2, its effects on FGFR2 activity was then investigated.

FIG. 40 is an autoradiogram illustrating the effects of C1GALT1 on the tyrosine phosphorylation of FGFR2 in colorectal cancer cells in accordance with an embodiment of the present invention C1GALT1 overexpressing HCT116 and SW480 cells and C1GALT1 knocked down HCT116 and SW620 cells were serum starved and treated with (+) or without (−) bFGF 25 ng/ml for 5 minutes. Cell lysates were then immunoprecipitated with anti-FGFR2 antibody and blotted with anti-phosphotyrosine (4G10) or anti-FGFR2 antibodies. Our results indicated that overexpression of C1GALT1 (C1GALT1) increased while knockdown of C1GALT1 (shC1GALT1) suppressed FGFR2 tyrosine phosphorylation after bFGF treatment, as shown in FIG. 40. These findings suggest that C1GALT1 modifies O-glycans on FGFR2 and regulated bFGF-induced activation of FGFR2.

### Breast Cancer

Breast cancer is the most diagnosed malignancy in women being the fourth cause of cancer-related death in Taiwan. Approximately 10-15% of patients with breast cancer have an aggressive disease and develop distant metastases within 3 years after the initial detection of primary tumor. However, effective therapies to reduce metastasis or recurrence remain a critical challenge.

### Analysis Results of Effects of C1GALT1 Inhibition on Breast Cancer

Up-Regulation of C1GALT1 Correlates with Breast Cancer Histological Grade and Advanced Tumor Stage:

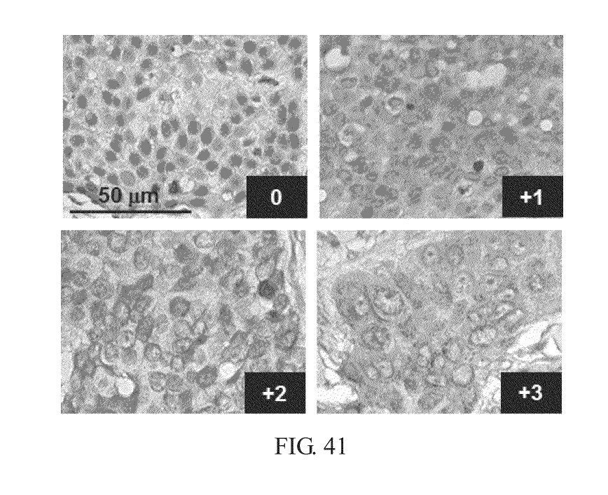

FIG. 41 is an image illustrating C1GALT1 immunohistochemistry staining intensity scores of breast cancer tissue microarray in accordance with an embodiment of the present invention Immunohistochemistry of breast cancer tissue microarray (BRC1021, Pantomics Inc.) visualized with 3,3-diaminobenzidine liquid substrate system and counterstained with hematoxylin reveals that C1GALT1 expression is similar to intracellular localization of Golgi apparatus. The intensity of staining was scored according to the percentage of C1GALT1-positive cells in each tissue (0, negative; +1<20%, +2, 20-50%, +3, >50%), as shown in FIG. 41.

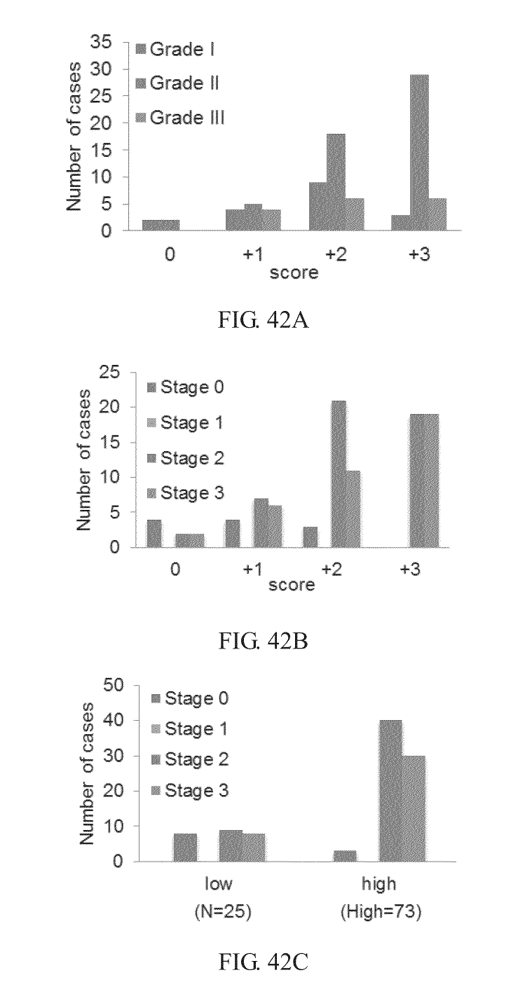

FIGS. 42A-42B are schematic diagrams illustrating the C1GALT1 staining intensity scores against the breast cancer histological grades and the tumor stages in accordance with an embodiment of the present invention. C1GALT1 staining intensity is scored from 0 to +3 and plotted against histological grade, as shown in FIG. 42A, and tumor stage as shown in FIG. 42B. FIG. 42C is a schematic diagram illustrating the C1GALT1 expression levels against the tumor stages in accordance with an embodiment of the present invention. The low expression group comprises scoring from 0 to +1 and the high expression group comprises scoring from +2 to +3. Spearman Rank Correlation reveals that C1GALT1 expression is correlated with histological grade and tumor stage, and Chi-square shows higher C1GALT1 expression levels (+2 and +3; 71.4%, n=70) correlates with advanced tumor stage as shown in Table 2. C1GALT1 up-regulation correlates with histological grade, advanced tumor stage in breast cancer.

TABLE 2
C1GALT1 expression level correlates with clinicopathological
characteristics in breast cancer:

| | C1GALT1 expression | | | | |
|---|---|---|---|---|---|
| Factor | 0 | +1 | +2 | +3 | p value |
| Grade I | 2 | 4 | 9 | 3 | **p < 0.01 |
| Grade II | 2 | 6 | 18 | 29 | Spearman |
| Grade III | 0 | 4 | 6 | 6 | Rank |
| | | | | | Correlation |
| Stage 0 | 4 | 4 | 3 | 0 | **p < 0.01 |
| Stage 1 | 0 | 0 | 0 | 0 | Spearman |
| Stage 2 | 2 | 7 | 21 | 19 | Rank |
| Stage 3 | 2 | 6 | 11 | 19 | Correlation |
| | Low (N = 25) | High (N = 73) | | | |
| Stage 0 | 8 | 3 | | | **p < 0.01 |
| Stage 1 | 0 | 0 | | | Chi-square |
| Stage 2 | 9 | 40 | | | |
| Stage 3 | 8 | 30 | | | |

*p < 0.05;
**p < 0.01

### C1GALT1 is Differentially Expressed in Multiple Breast Cancer Cell Lines:

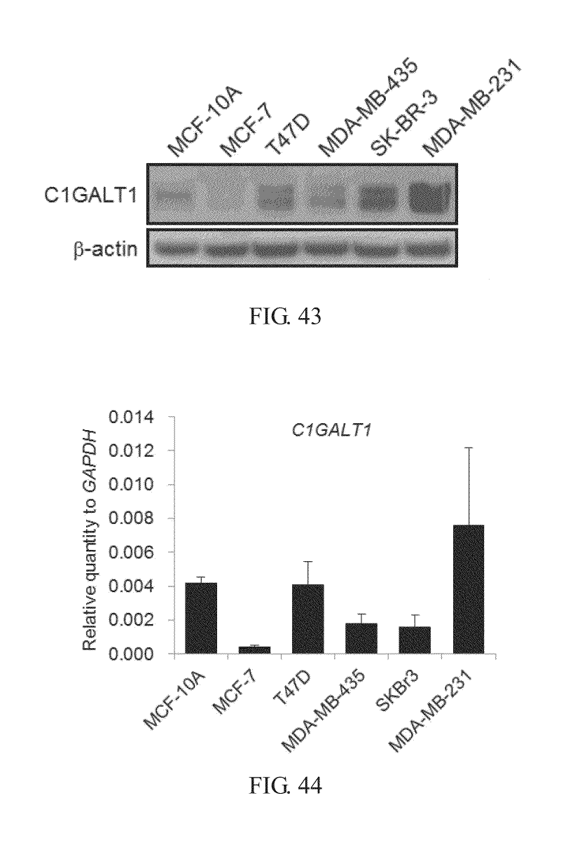

FIG. 43 is an autoradiogram illustrating C1GALT1 expression in six breast cancer cell lines analyzed by Western blotting in accordance with an embodiment of the present invention. FIG. 44 is an autoradiogram illustrating C1GALT1 expression in six breast cancer cell lines analyzed by quantitative real-time RT-PCR in accordance with an embodiment of the present invention. Multiple breast cancer cell lines MCF-10A, MCF-7, T47D, MDA-MB-435, SK-BR-3 and MDA-MB-231 were used to test for C1GALT1 mRNA and protein expression levels by Q-PCR and Western blot. C1GALT1 mRNA and protein is differentially expressed in different breast cancer cell lines, as shown in FIGS. 43 and 44. C1GALT1 mRNA and protein expression is higher in MDA-MB-231 and T47D cells and lower in MCF-7 and MCF-10A, as shown in FIGS. 43 and 44.

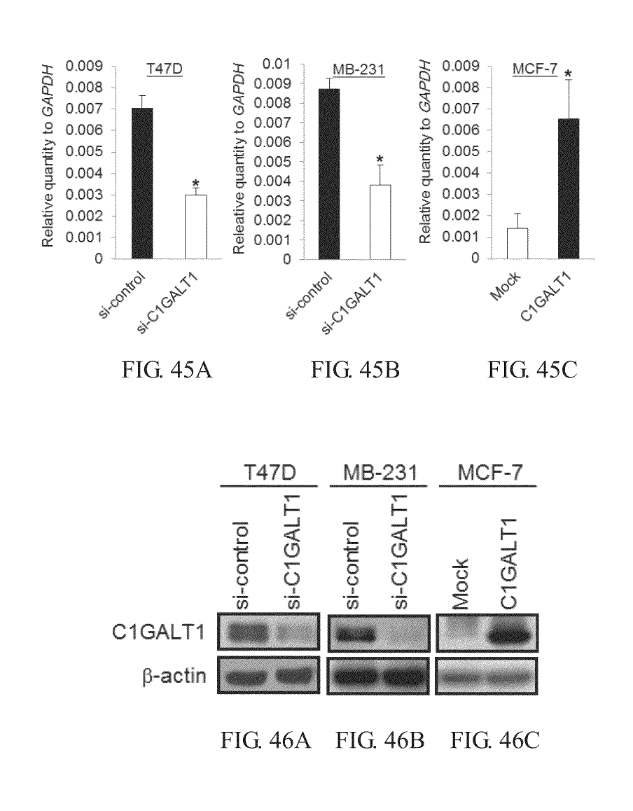

FIGS. 45A-45B are schematic diagrams illustrating C1GALT1 expression analyzed by quantitative real-time RT-PCR in breast cancer cell lines of a control group (si-control) and a C1GALT1 knockdown group (si-C1GALT1) in accordance with an embodiment of the present invention. FIG. 45C is a schematic diagram illustrating C1GALT1 expression analyzed by quantitative real-time RT-PCR in breast cancer cell lines of a control group (Mock) and a C1GALT1 overexpression group (C1GALT1) in accordance with an embodiment of the present invention. FIGS. 46A-46B are schematic diagrams illustrating C1GALT1 expression analyzed by Western blotting in breast cancer cell lines of a control group (si-control) and a C1GALT1 knockdown group (si-C1GALT1) in accordance with an embodiment of the present invention. FIG. 46C is a schematic diagram illustrating C1GALT1 expression analyzed by Western blotting in breast cancer cell lines of a control group (Mock) and a C1GALT1 overexpression group (C1GALT1) in accordance with an embodiment of the present invention. Knockdown of C1GALT1 mRNA and protein by C1GALT1 specific siRNA and non-target siRNA (Invitrogen) in MDA-MB-231 and T47D cells with Lipofectamine® RNAiMAX (Invitrogen) were confirmed by Q-PCR and Western blot, as shown in FIGS. 45A-45B and FIGS. 46A-46B Overexpression of C1GALT1 in MCF-7 was achieved by transfection of vector and C1GALT1/pcDNA3.1 plasmid with Lipofectamine® 2000 (Invitrogen), confirmed by Q-PCR, as shown in FIG. 45C and Western blot, as shown in FIG. 46C.

### C1GALT1 Expression Regulates Cancer Malignant Behaviors:

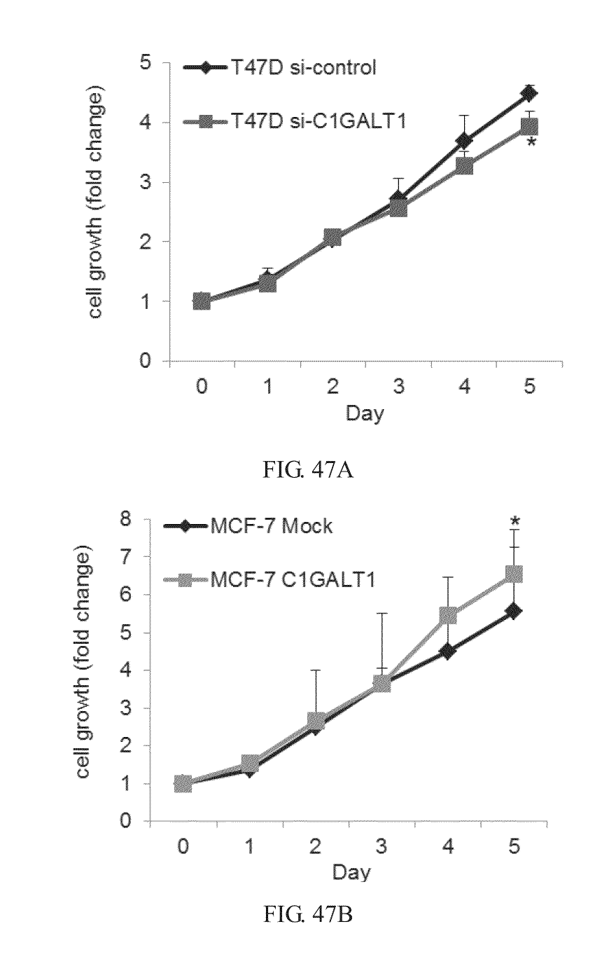

FIGS. 47A-47B are graphs illustrating the effects of C1GALT1 on cell viability of breast cancer cells by MTT assay in accordance with an embodiment of the present invention. Control or C1GALT1 knocked down T47D cells (2×10³) were seeded into 96-well plate and incubated with 10% FBS in DMEM medium from day 0-5. The results were analyzed by spectrophotometer and normalized to day 0. MTT assays revealed that C1GALT1 knockdown (si-C1GALT1) decreased cell viability in T47D cells compared with control, while C1GALT1 overexpression (C1GALT1) enhanced cell viability in MCF-7 cells compared with mock, as shown in FIGS. 47A-47B.

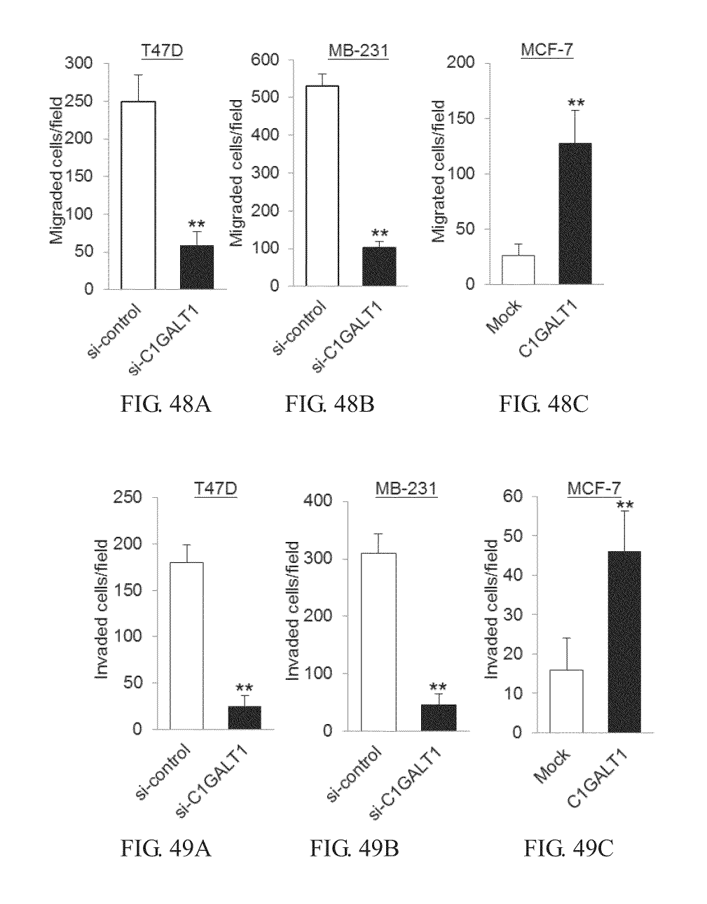

FIGS. 48A-48C are schematic diagrams illustrating the effects of C1GALT1 on migration of breast cancer cells in accordance with an embodiment of the present invention. Control or C1GALT1 knocked down T47D and MB-231 cells and mock or C1GALT1 overexpressed MCF-7 cells (5×10⁴) in serum free DMEM medium were seeded into the upper chamber of transwell, and 10% FBS in DMEM was added to the lower chamber as a chemoattractant for migration assay. FIGS. 49A-49C are schematic diagrams illustrating the effects of C1GALT1 on invasion of breast cancer cells in accordance with an embodiment of the present invention. Matrigel™ (BD Biosciences) transwell was used in invasion assay. The number of migrated and invaded cells was counted from three fields under microscopy. Data are represented as mean±SD from three independent experiments. *p<0.05; **p<0.01. C1GALT1 knockdown (si-C1GALT1) suppressed T47D and MB-231 cell migration and invasion. C1GALT1 overexpression (C1GALT1) promoted MCF-7 cell migration and invasion, as shown in FIGS. 48A-48C and FIGS. 49A-49C. These results indicated that C1GALT1 modulates cancer malignant phenotypes in cell viability, migration and invasion. C1GALT1 expression regulates cancer stem cell-like marker and stemness phenotypes in breast cancer:

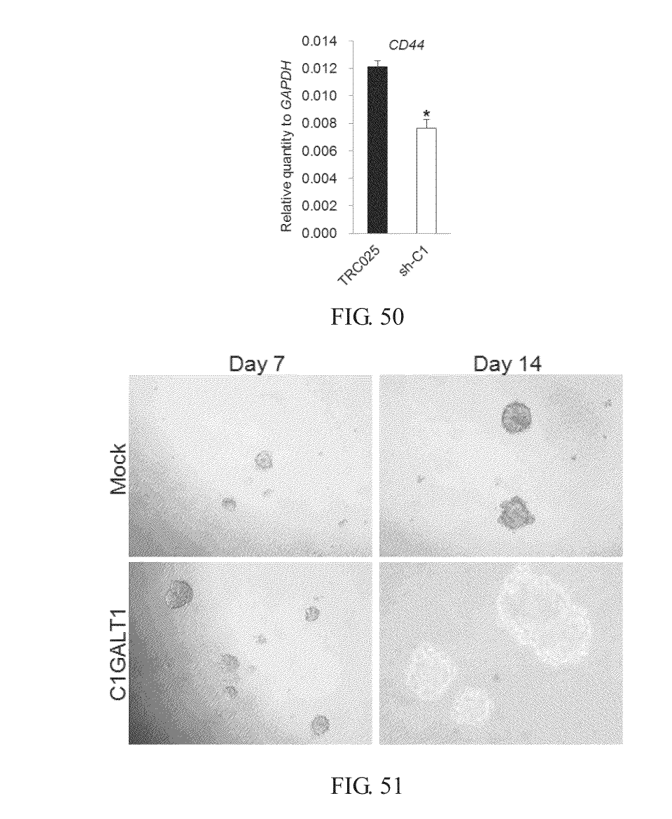

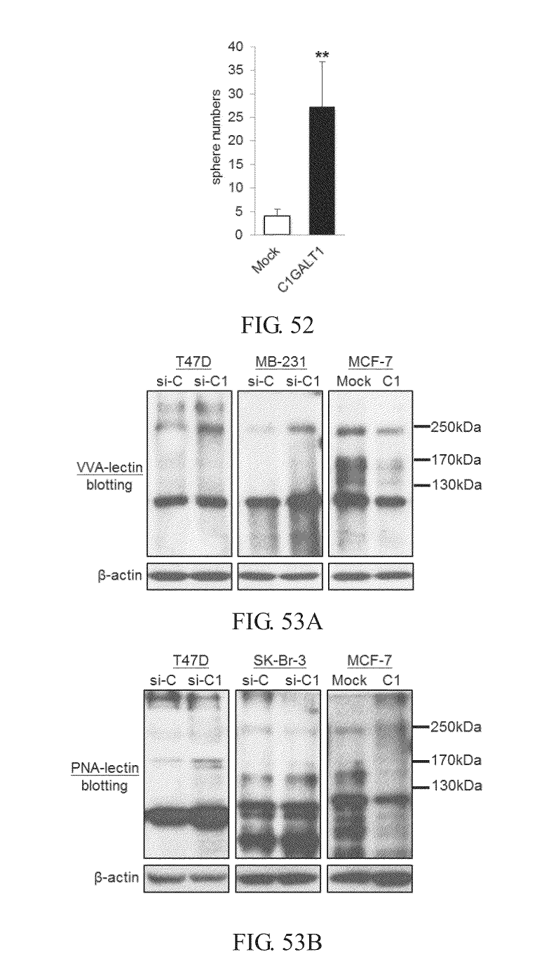

FIG. 50 is a schematic diagram illustrating the effects of C1GALT1 on stem-cell markers in breast cancer cells in accordance with an embodiment of the present invention. Knockdown of C1GALT1 in T47D cells by shRNA confirmed by Q-PCR normalized to GAPDH. Cancer stem cells posses self-renewal, oncogenesis and highly metastatic properties in breast cancer. Several cancer stem cell-like related markers have been identified, for example, CD44, CD24, Oct-4, Naong and SOX-2. In breast cancer, CD44+/CD24− is a classical marker of molecular switch from cancer cells to cancer stem cells and up-regulation of CD44+/CD24− is involved in cancer tumorigenesis and metastasis. The data showed that knockdown of C1GALT1 (sh-C1) suppressed cancer stem cell marker CD44 mRNA expression in T47D cells. FIG. 51 is an image illustrating the effects of C1GALT1 on sphere formation of breast cancer cells in accordance with an embodiment of the present invention. FIG. 52 is a schematic diagram illustrating the effects of C1GALT1 on sphere formation of breast cancer cells in accordance with an embodiment of the present invention. C1GALT1 overexpression in MCF-7 cells 1×10³ seeded into 24-well ultra-low attachment plate with serum free DMEM containing 20 ng/ml EGF, 10 ng/ml FGF and 1×B27 supplement medium. The images of sphere formation were taken by a Nikon camera at day 7 and 14, and the number of spheres (diameter >100 μm) was counted. Data are presented as mean±SD from three independent experiments. *p<0.05; **p<0.01. Overexpression of C1GALT1 (C1GALT1) enhanced sphere formation in MCF-7 cells, as shown in FIGS. 50-52 This data suggest that C1GALT1 could regulate breast cancer stemness properties.

### C1GALT1 Regulates O-Glycosylation in Breast Cancer Cell Lines:

FIGS. 53A-53B are autoradiograms illustrating the effects of C1GALT1 on O-glycosylation of glycoproteins in breast cancer cell lines in accordance with an embodiment of the present invention. The effects of C1GALT1 glycosylation in breast cancer were analyzed by Western blot. Total cell lysates collected from C1GALT1 knocked down T47D and MB-231 cells, and C1GALT1 overexpressed MCF-7 cells were subjected to immunoblotting. Biotinylated Vicia Villosa agglutinin (VVA) and peanut agglutinin (PNA) lectin were used to detect and pull down Tn and T antigen. VVA is specific for α- or β-GalNAc (Tn antigen), and PNA is specific for galactosyl β-1,3-GalNAc-Gal (T antigen). Streptavidin was used as secondary antibody. C1GALT1 knockdown (si-C1) increased Tn antigen in T47D and MB-231 cells while C1GALT1 overexpression (C1) decreased Tn antigen expression in MCF-7 cells, as shown in FIG. 53A. Similarly, knockdown of C1GALT1 decreased T antigen expression (si-C1) in T47D and SK-BR-3 and overexpression of C1GALT1 (C1) increased T antigen expression in MCF-7, as shown in FIG. 53B.

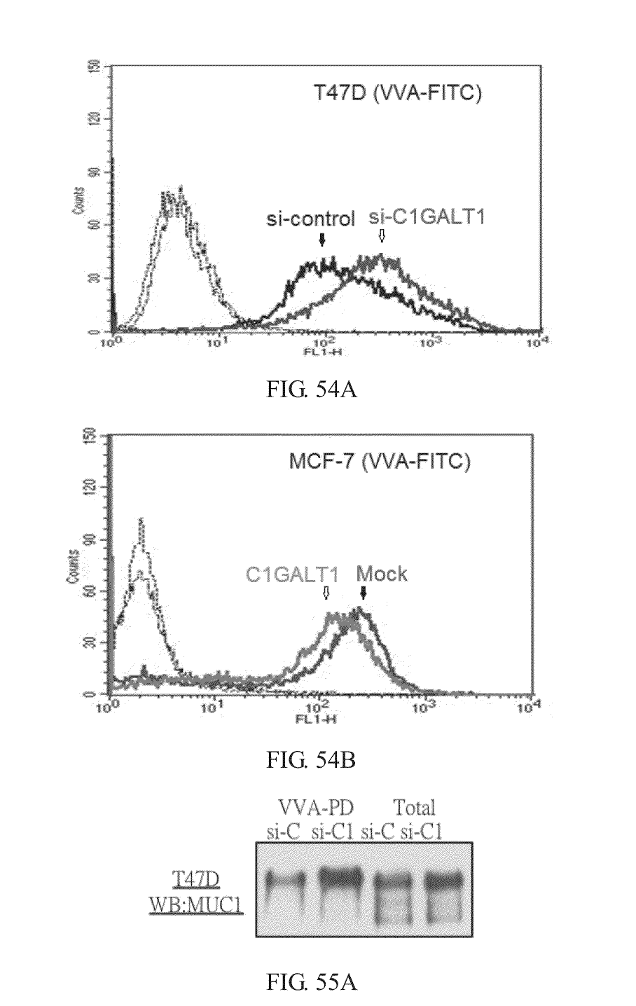

FIGS. 54A-54B are graphs illustrating surface O-glycans of breast cancer cell surfaces analyzed by flow cytometry in accordance with an embodiment of the present invention. C1GALT1 knockdown and overexpressed cells (1×10⁵) pre-treated with neuraminidase for 30 min and immunofluorescently labeled VVA and PNA lectin incubated with transfectants for 30 min on ice and PBS washed twice. The results were analyzed by BD FACS Calibur. Flow cytometry data show that C1GALT1 knockdown in T47D cells (si-C1GALT1) increased cell surface Tn antigen expression, as shown in FIG. 54A, while C1GALT1 overexpression in MCF-7 (C1GALT1) decreased cell surface T antigen, as shown in FIG. 54B. Surprisingly, C1GALT1 knocked down T47D and MB-231 cells (si-C1GALT1) show increased VVA binding above 130 kDa but decreased PNA binding in T47D and SK-BR-3 cells above 130 kDa. Similarly, C1GALT1 overexpression in MCF-7 cells (C1GALT1) decreased VVA binding and increased PNA binding above 130 kDa. C1GALT1 glycosylates MUC1 oncoprotein in breast cancer cell lines:

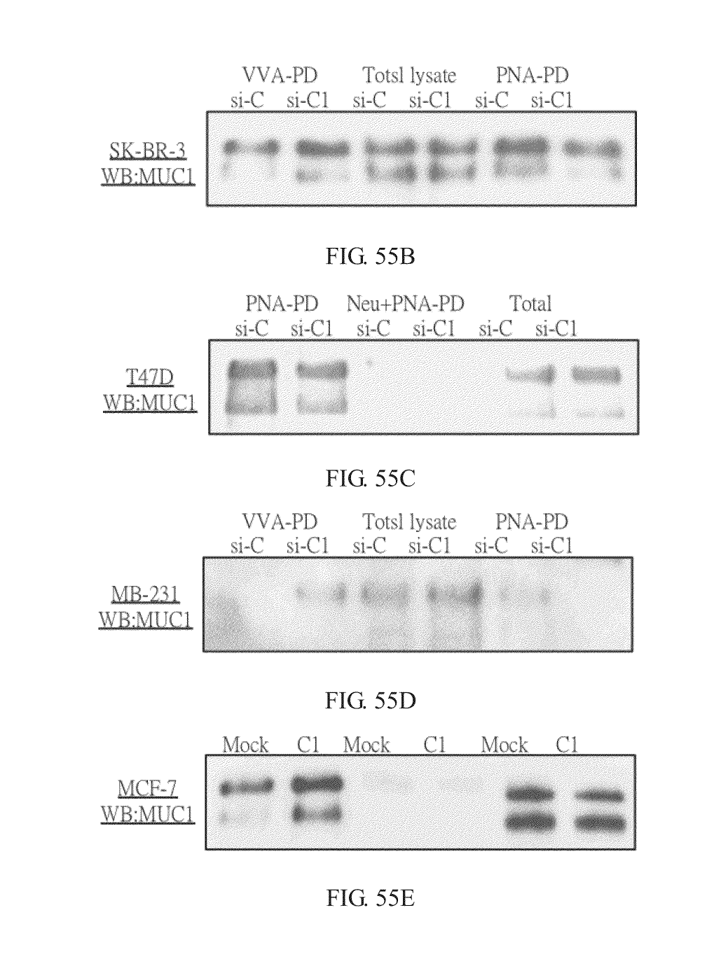

C1GALT1 glycosylates glycoproteins from molecular weight 130 kDa and above, as shown in FIGS. 53A-53B and FIGS. 54A-54B. Next, the possible candidates, MUC1 (above 250 kDa), a critical molecule in breast cancer glycosylated by C1GALT1 is tested. FIGS. 55A-55E are autoradiograms illustrating the effects of C1GALT1 on O-glycosylation on MUCI in breast cancer cells in accordance with an embodiment of the present invention. C1GALT1 expressions in T47D, SK-BR-3 and MB-231 cells were knocked down, and C1GALT1 is overexpressed in MCF-7 cells. Total cell lysates (300 μg) of T47D, SK-BR-3, MB-231 and MCF-7 cells were incubated with VVA- and PNA-lectin beads to immunoprecipitate Tn and T antigen, and monoclonal anti-MUC1 antibody was used to detect MUC1 by Western blot. Results showed that knockdown C1GALT1 (si-C1) enhanced VVA-lectin binding and decreased in PNA-lectin binding of MUC1. Overexpression of C1GALT1 (C1) decreased VVA-lectin binding and increased PNA-lectin binding of MUC1, as shown in FIGS. 55A-55E. Thus, knockdown of C1GALT1 (si-C1) increased Tn antigen expression on MUC1 oncoprotein and decreased T antigen on MUC1 oncoprotein in T47D, SK-BR-3 and MB-231 transfectants. Overexpression of C1GALT1 (C1) enhanced T antigen expression on MUC1 in MCF-7 transfectants. This data indicated that C1GALT1 modified the O-glycans on MUC1 oncoprotein in breast cancer.

### C1GALT1 Promotes Breast Cancer Cell Tumorigenesis In Vivo:

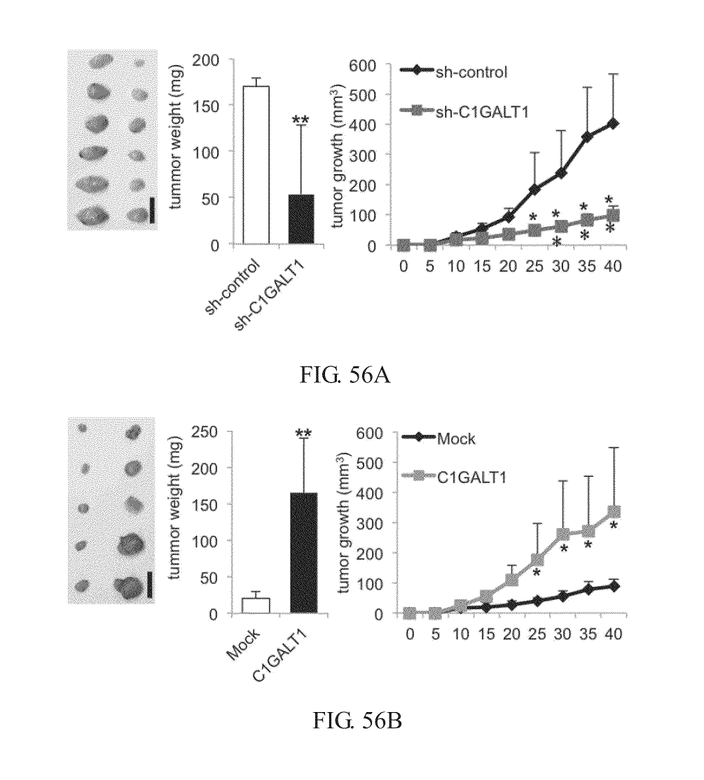

FIGS. 56A-56B includes an image, a schematic diagram, and a graph illustrating the effects of C1GALT1 on tumor growth of breast cancer cells in accordance with an embodiment of the present invention. Stable knockdown or overexpression of C1GALT1 in T47D and MCF-7 cells was achieved by control (sh-control) or C1GALT1 (shC1GALT1) shRNA and vector (Mock) or C1GALT1/pcDNA3.1 (C1GALT1) plasmids. Cells (5×10⁶) were prepared and mixed with Matrigel 1:1 for mice xenograft. Tumor growth was measured every 5 days and animals were scarified after 40 days. Data are presented as mean±SD from three independent experiments. *p<0.05; **p<0.01. Stable knockdown of C1GALT1 with shRNA (shC1GALT1) suppressed cell growth while overexpression of C1GALT1 (C1GALT1) increased cell growth in breast cancer cell lines analyzed by MTT assay, as shown in FIGS. 56A-56B. Xenograft animal models showed that knockdown of C1GALT1 (shC1GALT1) suppressed tumor growth and tumor weight in T47D shC1GALT1 transfectant compared with TRC025 transfectant, and overexpression of C1GALT1 (C1GALT1) promoted tumor growth and tumor weight in MCF-7 C1GALT1 transfectant compare with Mock transfectant. This result indicated that C1GALT1 promotes tumorigenesis in breast cancer.

### Itraconazole Impairs C1GALT1 Function Leading to the Accumulation of Tn Antigen Expression in Breast Cancer Cells:

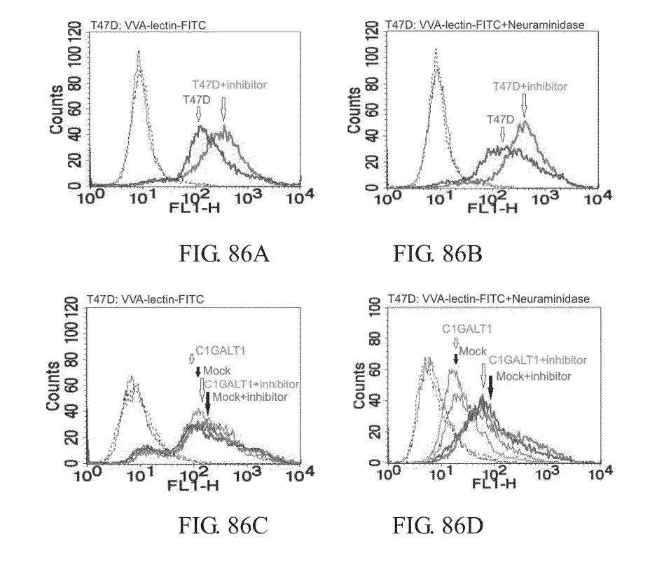

FIGS. 86A-86D are graphs illustrating that itraconazole impairs C1GALT1 function leading to the accumulation of Tn antigen expression in breast cancer cells in accordance with an embodiment of the present invention. To confirm itraconazole binding to the catalytic domain of C1GALT1, the inventors conducted in vitro studies and revealed whether itraconazole impairs C1GALT1 function leading to Tn antigen accumulation. As shown in FIG. 86A, T47D parental cells are treated with (indicated by a long white arrow) or without (indicated by a short white arrow) itraconazole 10 ug/ml. After itraconazole treatment, cells are detached with 5 mM EDTA. Afterwards, FITC conjugated Vicia villosa agglutinin (VVA), lectin specifically recognizing Tn antigen, is applied for analysis with a fluoresencce-activated cell sorting (FACS) system. A right-shift of the VVA peak indicates the accumulation of Tn antigens. As shown in FIG. 86B, T47D parental cells are treated with (indicated by a long white arrow) or without (indicated by a short white arrow) itraconazole 10 ug/ml. After itraconazole treatment, cells are detached with 5 mM EDTA and treated with or without 10 mU/ml neuraminidase to unmask the effects of sialylated O-glycans. After treatment with neuraminidase, FITC conjugated Vicia villosa agglutinin (VVA), lectin specifically recognizing Tn antigen, is applied for analysis with a fluoresencce-activated cell sorting (FACS) system. Our results show that itraconazole treatments lead to a right shift of the VVA peak regardless of the presence of sialylated O-glycans in the parental cells of T47D cell lines. To confirm whether the peak shift is a result of impaired C1GALT1 function, mock and C1GALT1 overexpressed T47D cells are analyzed. FIG. 86C shows mock (indicated by black arrows) and C1GALT1 (indicated by white arrows) overexpressed T47D cells with (indicated by long arrows) or without (indicated by short arrows) itraconazole 10 ug/ml treatment. Overexpression of C1GALT1 results in a left-shift of the VVA peak in itraconazole-untreated transfectants, indicating intact C1GALT1 function in depleting Tn antigens as substrates for T antigen synthesis. Treatment of itracozole results in a major right-shift of the VVA peaks in both mock and C1GALT1 overexpressed transfectants, indicating impaired C1GALT1 function leading to accumulation of Tn antigens. As shown in FIG. 86D, similar results are seen in mock and C1GALT1 overexpressed transfectants with sialylated O-glycans unmasked by neuraminidase. Overexpression of C1GALT1 in the cell line shows peak shifts to the left regardless of the presence of sialylated O-glycans compared with mock transfectants, indicating increased T antigen formation by depleting the available Tn antigen substrate. Treatment with itraconazole resulted in major peak shifts to the right in C1GALT1 overexpressed cells, denoting the accumulation of Tn antigen substrates and signifying impaired C1GALT1 function.

### Itraconazole Suppresses Breast Cancer Cell Growth:

FIG. 88B is a schematic diagram illustrating that itraconazole suppresses breast cancer cell growth in accordance with an embodiment of the present invention. To determine whether impairing C1GALT1 function affects breast cancer cell malignant phenotypes, the effects of itraconazole on breast cancer cell growth are analyzed by MTT cell growth assay in T47D cell lines. Cells (3×10³) are seeded into 96-well microtitre plates and treated with DMSO (control) or itraconazole 10 g/ml for 5 days at 37° C. under 5% CO₂ atmosphere. Results from two independent experiments are presented as mean±SD and analyzed with student's t-test, *p<0.05 and **p<0.01. FIG. 88B shows that breast cancer cell growth is significantly suppressed by itraconazole compared with the control (DMSO).

### Head and Neck Squamous Cell Carcinoma

Head and neck squamous cell carcinoma (HNSCC) is the 10^{th} most frequently diagnosed cancers in males worldwide. Over 60% of new cases are diagnosed in developing countries and more than 50% of these cases die of cancer annually. Males are affected more significantly than females with a ratio of 2:1 to 4:1. The risk factors most closely associated with HNSCC are tobacco smoking, alcohol consumption, human papillomavirus (HPV), and Ebstein-Barr virus (HBV) infection

### C1GALT1 Expression is Up-Regulated in HNSCC:

FIG. 57 is a schematic diagram of illustrating mRNA expression of C1GALT1 in head and neck squamous cell carcinoma (HNSCC). A refers to the fold change of C1GALT1 expression in tongue carcinoma from patients with stage N1 disease compared with the healthy tongue controls (Fold change: 1.36). B refers to the fold change of C1GALT1 expression in the tongue carcinoma from patients with stage N1 disease compared with stage NO (Fold change: 1.32). C refers to the fold change of C1GALT1 expression in the nasopharyngeal carcinoma T1N1 compared with the nasopharyngeal carcinoma T1N0 (Fold change: 2.15).

### C1GALT1 Regulates HNSCC Protein O-Glycosylation:

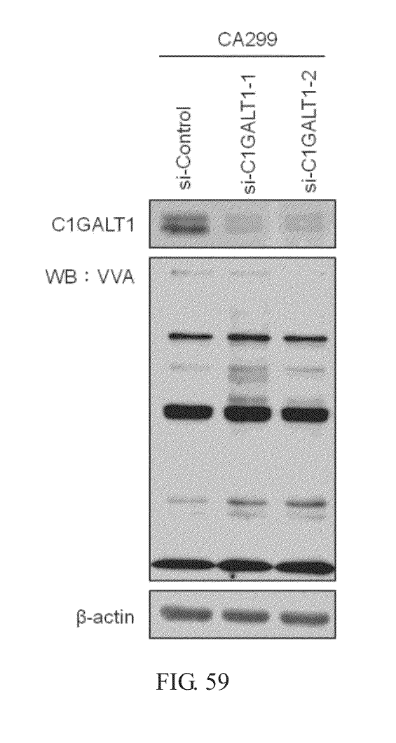

FIG. 58 is an autoradiogram illustrating C1GALT1 expression in three head and neck squamous cell carcinoma (HNSCC) cell lines analyzed by Western blotting in accordance with an embodiment of the present invention. FIG. 59 is an autoradiogram illustrating C1GALT1 expression and the effects of C1GALT1 on O-glycosylation of glycoproteins in head and neck squamous cell carcinoma (HNSCC) of a control group and C1GALT1 knockdown groups in accordance with an embodiment of the present invention. C1GALT1 knockdown with non-targeting (si-Control) or C1GALT1 specific siRNA-1 (si-C1GALT1-1) or C1GALT1 specific siRNA-2 (si-C1GALT1-2) in CA299 was confirmed by Western blot. Total cell lysates were collected and immunobloted with anti-VVA antibody. Western blot of total cell lysates from control or C1GALT1 knockdown CA299 cells with non-targeting or C1GALT1 specific siRNAs immunobloted with anti-VVA antibody indicates an increased Tn antigen expression on total glycoproteins in CA299 cells.

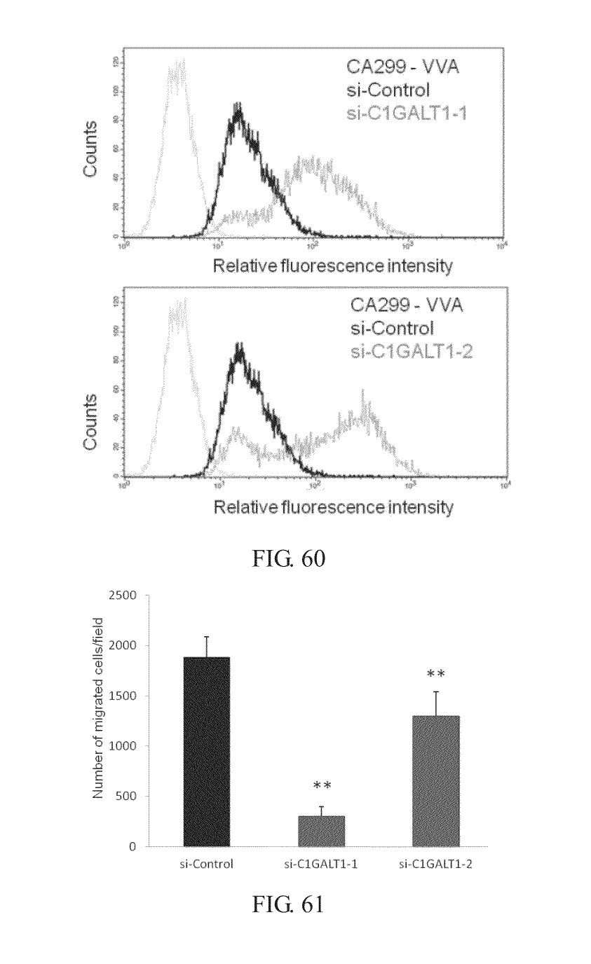

FIG. 60 is a graph illustrating surface O-glycans of HNSCC cell surfaces analyzed by flow cytometry in accordance with an embodiment of the present invention. Knockdown of C1GALT1 (si-C1GALT1-1, si-C1GALT1-2) in CA299 cells also resulted in increased Tn antigen expression on cell surface glycoproteins confirmed by flow cytometry.

### C1GALT1 Knockdown Suppresses CA299 Cell Migration.

FIG. 61 is a schematic diagram illustrating the effect of C1GALT1 on migration of HNSCC in accordance with an embodiment of the present invention. The effects of C1GALT1 knockdown on HNSCC malignant phenotype was investigated using transwell migration assays. CA299 cells were transfected with non-targeting (si-Control) or C1GALT1 specific (si-C1GALT1-1 and si-C1GALT1-2) siRNA. Cells (5×10⁴) in serum free DMEM were loaded into the upper chamber and serum free DMEM (SF) with FBS 10% were loaded to the lower chamber of the transwell migration systems. After 24 h the number of migrated cells from 5 random fields was counted under the microscope. Results obtained were analyzed by student's t-test and graphed by mean±SD, **p<0.05. The number of migrated cells was significantly less in C1GALT1 knockdown CA299 cells compared with control, as shown in FIG. 61.

### Lung Cancer

Lung cancer, also known as pulmonary carcinoma, is the leading cause of cancer mortality worldwide. Base on GLOBOCAN 2012 estimates, lung cancer incidence accounts for 13% of 14.1 million cancer diagnoses and 19% of 8.2 million cancer-caused deaths globally.

### C1GALT1 Expression is Up-Regulated in Lung Cancer:

FIG. 62 is a schematic diagram illustrating the mRNA expression of C1GALT1 in lung adenocarcinoma and normal lung tissues. Data analyzed from a public database (246 samples, Oncomine) shows a 2.569 fold increase of C1GALT1 mRNA expression levels in lung adenocarcinoma (B, n=226) compared with normal lungs (A, n=20), *p<0.01.

FIG. 63 is a schematic diagram illustrating fold changes of mRNA expression of C1GALT1 in 8 different biosets. Publically available resources (NextBio Research) analyzing 8 different biosets depict increased C1GALT1 mRNA expression levels in various fold changes with the highest expression level shown in lung adenocarcinoma tumors of stage 1 patients vs. adjacent normal tissue (fold change 3.1), *p<0.01. A refers to the fold change of C1GALT1 expression in human lung adenocarcinoma compared with normal lung tissues (Fold change: 1.41). B refers to the fold change of C1GALT1 expression in human squamous cell lung carcinoma compared with normal lung tissues (fold change: 1.28). C refers to the fold change of C1GALT1 expression in lung adenocarcinomas compared with adjacent normal controls (fold change: 2.37). D refers to the fold change of C1GALT1 expression in lung adenocarcinoma tumor of stage 1 compared with adjacent normal tissues (fold change: 3.1). E refers to the fold change of C1GALT1 expression in lung adenocarcinoma primary tumor compared with normal lung tissues (fold change 2.54). F refers to the fold change of C1GALT1 expression in lung cancer of stage 3 from Taiwanese female non-smoker patients compared with adjacent normal lung tissues (fold change 2.86). G refers to the fold change of C1GALT1 expression in lung cancer of stage 1 Taiwanese female non-smoker patients compared with adjacent normal lung tissues (fold change 1.85). H refers to the fold change of C1GALT1 expression in lung cancer of stage 2 Taiwanese female non-smoker patients compared with adjacent normal lung tissues (fold change 1.6). C1GALT1 expression regulates lung cancer cell malignant behavior:

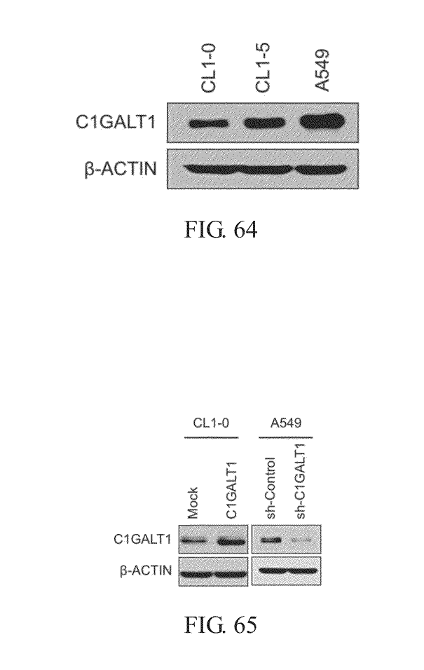

FIG. 64 is an autoradiogram illustrating C1GALT1 expression in three lung cancer cell lines analyzed by Western blotting in accordance with an embodiment of the present invention. C1GALT1 is differentially expressed in lung cancer cells lines, CL1-0, CL1-5, and A549. FIG. 65 is an autoradiogram illustrating the overexpression and knockdown of C1GALT1 in lung cancer cell lines confirmed by Western blotting in accordance with an embodiment of the present invention. Mock and C1GALT1 overexpression in CL1-0 cells and control (sh-Control) and C1GALT1 knockdown (sh-C1GALT1) in A549 cells are confirmed by Western blot. CL1-0 cells with comparatively lower C1GALT1 expression levels are stably transfected with pcDNA3.1A/vector or pcDNA3.1A/C1GALT1 plasmids to overexpress C1GALT1, and cells are selected and maintained with 400 ug/ml G418. A549 cells with comparatively higher C1GALT1 expression levels are stably transfected with control (sh-Ctrl) or C1GALT1 short-hairpin RNA (sh-C1GALT1) to stably knockdown C1GALT1 expression. Cells are selected and maintained with 2 ug/ml puromycin.

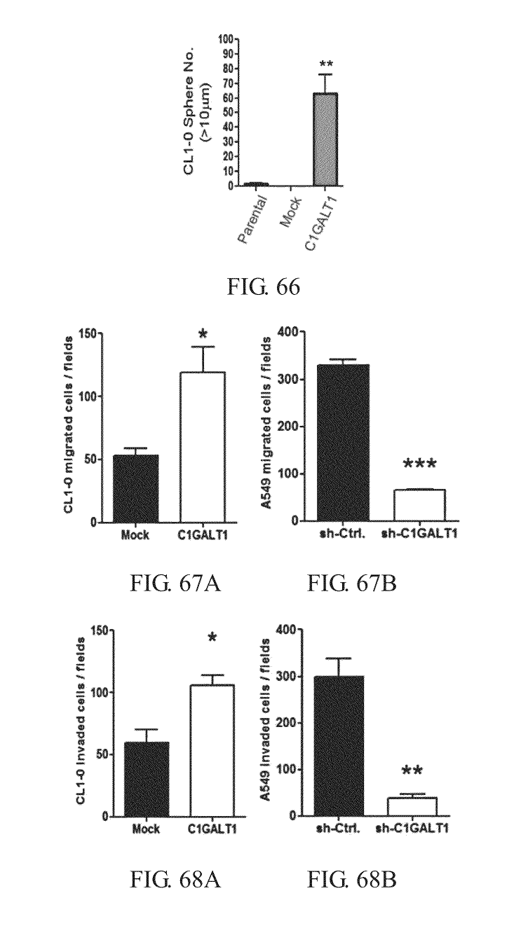

FIG. 66 is a schematic diagram illustrating C1GALT1 overexpression enhancing sphere formation in lung cancer cells in accordance with an embodiment of the present invention. FIGS. 67A-67B are schematic diagrams illustrating the effects of C1GALT1 on the migration of lung cancer cells in accordance with an embodiment of the present invention. FIGS. 68A-68B are schematic diagrams illustrating the effects of C1GALT1 on the invasion of lung cancer cells in accordance with an embodiment of the present invention. Cells (1×10⁴) are seeded into 24-well ultra-low attachment plate with serum free DMEM containing 20 ng/ml EGF, 10 ng/ml FGF, and 1×B27 supplement medium. The number of spheres formed (diameter >10 μm) is counted after 14 days under a microscope from 5 different fields. Results are presented as mean±SD from three independent experiments. Cells (2×10⁴-3×10⁴) in serum free DMEM are loaded into the upper chamber, and DMEM (SF) containing 10% FBS are loaded to the lower chamber of transwell migration or BioCoat Matrigel invasion chamber systems (Beckton Dickinson). Cells are serum starved overnight prior to seeding, and after 24 hours of migration and invasion, cells are fixed and stained with 0.5% (w/v) crystal violet (Sigma). The number of migrated or invaded cells from 5 random fields is counted under the microscope. Results obtained are analysed by student's t-test and graphed as percentage (%) of migrated or invaded cells relative to the control. C1GALT1 overexpression enhanced malignant phenotype behaviors in CL1-0. Overexpression of C1GALT1 enhanced sphere formation in CL1-0, as shown in FIG. 66. Overexpression of C1GALT1 enhanced CL1-0 cell migration, as shown in FIG. 67A, and invasion, as shown in FIG. 68A. **p<0.01. Knockdown of C1GALT1 suppressed A549 cell migration, as shown in FIG. 67B, and invasion, as shown in FIG. 68B. **p<0.01, and ***p<0.001.

### Itraconazole Impairs C1GALT1 Function Leading to Accumulation of Tn Antigen Expression in Lung Cancer Cells:

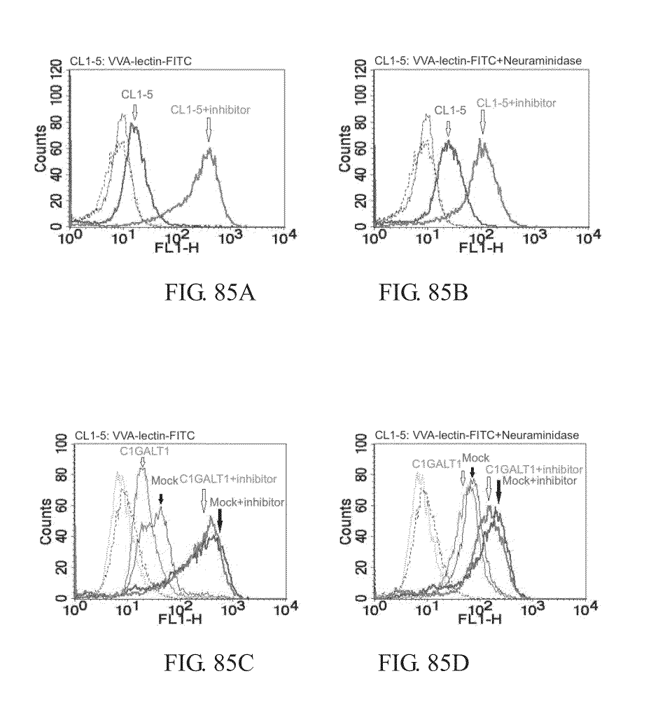

FIGS. 85A-85D are graphs illustrating that itraconazole impairs C1GALT1 function leading to the accumulation of Tn antigen expression in lung cancer cells in accordance with an embodiment of the present invention. To confirm Itraconazole binding to the catalytic domain of C1GALT1, the inventors conducted in vitro studies and revealed whether itraconazole impairs C1GALT1 function leading to Tn antigen accumulation. FIG. 85A shows CL1-5 parental cells treated with (indicated by a long white arrow) or without (indicated by a short white arrow) itraconazole 10 ug/ml. After itraconazole treatment, cells are detached with 5 mM EDTA. Afterwards, FITC conjugated Vicia villosa agglutinin (VVA), lectin specifically recognizing Tn antigen, is applied for analysis with a fluoresencce-activated cell sorting (FACS) system. A right-shift of the VVA peak indicates accumulation of Tn antigens. FIG. 85B shows CL1-5 parental cells treated with (indicated by a long white arrow) or without (indicated by a short white arrow) itraconazole 10 ug/ml. After itraconazole treatment, cells are detached with 5 mM EDTA and treated with or without 10 mU/ml neuraminidase to unmask the effects of sialylated O-glycans. After treatment with neuraminidase, FITC conjugated Vicia villosa agglutinin (VVA), lectin specifically recognizing Tn antigen, is applied for analysis with a fluoresencce-activated cell sorting (FACS) system. Our results show itraconazole treatment leads to a right shift of the VVA peak regardless of the presence of sialylated O-glycans in the parental cells of CL1-5 cell lines. To confirm whether the peak shift is a result of impaired C1GALT1 function, mock and C1GALT1 overexpressed CL1-5 cells are analyzed. FIG. 85C shows mock (indicated by black arrows) and C1GALT1 (indicated by white arrows) overexpressed CL1-5 cells with (indicated by long arrows) or without (indicated by short arrows) itraconazole 10 ug/ml treatment. Overexpression of C1GALT1 results in a left-shift of the VVA peak in itraconazole untreated transfectants, indicating intact C1GALT1 function in depleting Tn antigens as substrates for T antigen synthesis. Treatment of itracozole results in a major right-shift of the VVA peaks in both mock and C1GALT1 overexpressed transfectants, indicating impaired C1GALT1 function leading to the accumulation of Tn antigens. As shown in FIG. 85D, similar results are seen in mock and C1GALT1 overexpressed transfectants with sialylated O-glycans unmasked by neuraminidase. Overexpression of C1GALT1 in the cell line shows peak shifts to the left regardless of the presence of sialylated O-glycans compared with mock transfectants, indicating increased T antigen formation by depleting the available Tn antigen substrate. Treatment with itraconazole resulted in major peak shifts to the right in C1GALT1 overexpressed cells, denoting the accumulation of Tn antigen substrates and signifying impaired C1GALT1 function.

### Itraconazole Suppresses Lung Cancer Cell Growth:

FIG. 88A is a schematic diagram illustrating that itraconazole suppresses lung cancer cell growth in accordance with an embodiment of the present invention. To determine whether impairing C1GALT1 function affects lung cancer cell malignant phenotypes, the effects of itraconazole on lung cancer cell growth are analyzed by MTT cell growth assay in CL1-5 cell lines. Cells (3×10³) are seeded into 96-well microtitre plates and treated with DMSO (control) or itraconazole 10 g/ml for 5 days at 37° C. under 5% CO₂ atmosphere. Results from two independent experiments are presented as mean±SD and analyzed with student's t-test, *p<0.05 and **p<0.01. FIG. 88A shows that lung cancer cell growth is significantly suppressed by itraconazole compared with the control (DMSO).

### Ovarian Cancer

Ovarian cancer is seventh most common cancer diagnosis in women with global estimates of 239,000 new cases diagnosed each year. Ovarian cancer is the most lethal gynecological cancer, responsible for 140,000 deaths annually with approximately a 45% five year survival rate compared with 89% in breast cancer.

### Overexpression of C1GALT1 in Ovarian Cancer:

FIG. 69 is a schematic diagram illustrating the mRNA expression of C1GALT1 in various types ovarian cancers and normal ovarian tissues. Resources analyzed from a public database (Oncomine) show a significant increase in C1GALT1 expression in various types of ovarian cancers with the highest expression level displayed by ovarian mucinous adenocarcinoma (fold change 1.397), *p<0.001. A refers to the ovary. B refers to ovarian clear cell adenocarcinoma. C refers to ovarian endometrioid adenocarcinoma. D refers to ovarian mucinous adenocarcinoma. E refers to ovarian serous adenocarcinoma.

### Knockdown of C1GALT1 Suppresses Ovarian Cancer Cell Malignant Phenotype:

FIG. 70 is an autoradiogram illustrating C1GALT1 expression in three ovarian cancer cell lines analyzed by Western blotting in accordance with an embodiment of the present invention. Western blot displaying C1GALT1 is differentially expressed in the ovarian cancer cell lines, OVCAR3, SKOV3, and ES-2. FIG. 71 is an autoradiogram illustrating knockdown of C1GALT1 in an ovarian cancer cell line confirmed by Western blotting in accordance with an embodiment of the present invention. Western blot confirms knockdown of C1GALT1 with non-targeting (si-C) and C1GALT1 (si-C1) siRNA (20 nM) in ES-2 cells.

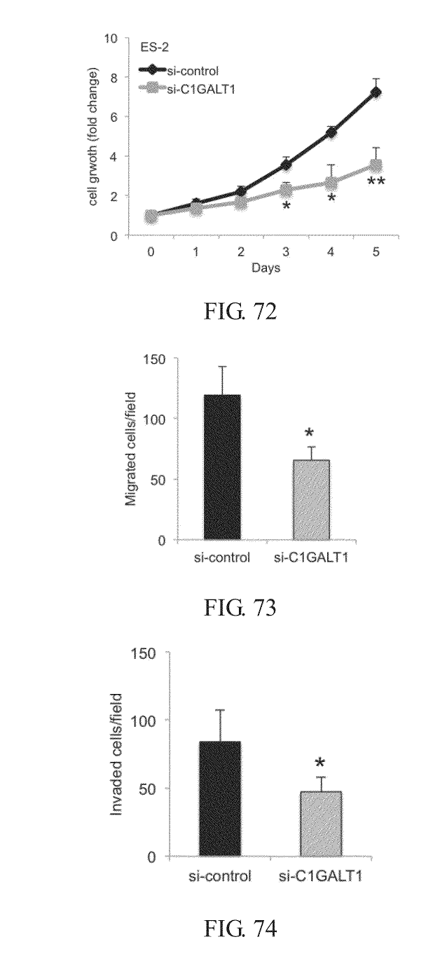

FIG. 72 is a schematic diagram illustrating the effects of C1GALT1 on the cell growth of ovarian cancer cells in accordance with an embodiment of the present invention. FIG. 73 is a schematic diagram illustrating the effects of C1GALT1 on the migration of ovarian cancer cells in accordance with an embodiment of the present invention. FIG. 74 is a schematic diagram illustrating the effects of C1GALT1 on the invasion of ovarian cancer cells in accordance with an embodiment of the present invention. Cells (3×10³) are seeded for MTT cell growth assay for 5 days. Results obtained are presented as mean cell growth in fold changes±SD from three independent experiments, *p<0.05 and **p<0.01 analysed by student's t-test. Cells (2×10⁴) in serum free DMEM are loaded into the upper chamber and DMEM with 10% FBS are loaded to the lower chamber of transwell migration or BioCoat Matrigel invasion chamber systems (Beckton Dickinson). Cells are serum starved overnight prior to seeding and after 24 hours of migration and invasion cells are fixed and stained with 0.5% (w/v) crystal violet (Sigma). The number of migrated or invaded cells from 5 random fields is counted under a microscope. Results obtained are analysed by student's t-test, *p<0.05. Knockdown of C1GALT1 suppressed ES-2 cell growth, as shown in FIG. 72, migration as shown in FIG. 73, and invasion as shown in FIG. 74.

### Endometrial Cancer

In comparison to ovarian cancer, endometrial cancer is the sixth leading cancer diagnosis in women with a global estimates of 319,605 new cases diagnosed in 2012 and an estimated 76,155 deaths occurring annually. Endometrial cancer is the most common gynecological cancer in developed countries and the second most common in developing countries.

### C1GALT1 Expression is Increased in Endometrial Cancer:

FIG. 75 is a schematic diagram illustrating mRNA fold changes of expression of C1GALT1 in fold changes in 3 different biosets. Selected biosets analyzed for C1GALT1 expression from NextBio Research indicate a number of gynecological cancers, including endometrial cancers, exhibit increased C1GALT1 expression level compared with normal tissues, *p<0.01. A. A refers to the fold change of C1GALT1 expression in endometrial cancer compared with non-cancerous atrophic tissue (fold change: 1.92). B refers to the fold change of C1GALT1 expression in HPV-positive cervical cancer compared with normal cervical tissue (fold change: 2.1). C refers to the fold change of C1GALT1 expression in heterologous carcinosarcoma compared with sarcoma (fold change: 1.63).

### Knockdown of C1GALT1 Suppresses Endometrial Cancer Cell Malignant Phenotype:

FIG. 76 is an autoradiogram illustrating C1GALT1 expression in two endometrial cancer cell lines analyzed by Western blotting in accordance with an embodiment of the present invention. C1GALT1 is differentially expressed in endometrial cell lines, HEC-1A and RL95-2. FIG. 77 is an autoradiogram illustrating knockdown of C1GALT1 in an endometrial cancer cell line confirmed by Western blotting in accordance with an embodiment of the present invention. Knockdown of non-targarting (si-Control) and C1GALT1 (si-C1GALT1) in RL95-2 cells with siRNA (20 nM) is confirmed by Western blot.

FIG. 78 is a schematic diagram illustrating the effects of C1GALT1 on the migration of endometrial cancer cells in accordance with an embodiment of the present invention. FIG. 79 is a schematic diagram illustrating the effects of C1GALT1 on the invasion of endometrial cancer cells in accordance with an embodiment of the present invention. Cells (2×10⁴) in serum free DMEM are loaded into the upper chamber, and DMEM with 10% FBS are loaded to the lower chamber of transwell migration or BioCoat Matrigel invasion chamber systems (Beckton Dickinson). Cells are serum starved overnight prior to seeding, and after 24 hours of migration and invasion cells are fixed and stained with 0.5% (w/v) crystal violet (Sigma). The number of migrated or invaded cells from 5 random fields is counted under a microscope. Results obtained are analysed by student's t-test and graphed as percentage (%) of migrated or invaded cell relative to the control. *P<0.05. Knockdown of C1GALT1 suppressed RL95-2 cell migration as shown in FIG. 78, and invasion as shown in FIG. 79.

### Cholangiocarcinoma

Cholangiocarcinomas (CCA) arise primarily from the epithelial lining of bile ducts (intrahepatic and extrahepatic bile duct) are relatively rare accounting for less than 1% of all cancers worldwide and 3% of all gastrointestinal cancer. High incidence rates are found in association with hepatolithiasis, hepatitis B and hepatitis C infection, and liver fluke (Opisthorchis viverrini or Clonorchis sinensis) infection with a reported incidence rate of 2.8/100 000 cases in Taiwan between 1998 to 2007. Cholangiocarcinoma is a predominantly lethal cancer, together with primary liver cancer, ranked the second most lethal cancers in Taiwan with a mortality rate of 24.95% in 2011.

Cholangiocarcinoma (CCA) is a Highly Lethal Cancer being the Fifth Most Common GALT1 Expression is Increased in CCA:

FIG. 80A is a bile duct tissue image illustrating different intensities of C1GALT1 staining in accordance with an embodiment of the present invention. Representative images of immunohistochemical staining of C1GALT1 with anti-C1GALT1 polyclonal antibody (Santa Cruz) of tissue microarray (Biomax BC03119 and Superbiochips CSA3) comprising normal bile ducts (normal) and CCA (tumor). C1GALT1 staining intensity in normal bile duct (N, n=6) and CCA (T, n=18) are scored from 0 to +3 and plotted against percentage of patient distribution (right panel). Scores 0 and +1 are considered low expression, and scores +2 and +3 are considered high expression.

FIG. 80B is a schematic diagram illustrating percentages of cases with different intensities levels of C1GALT1 immunohistochemistry staining in cholangiocarcinoma (CCA) and bile duct tissues in accordance with an embodiment of the present invention. Over 80% of CCA tissues exhibit high C1GALT1 expression levels, while all normal bile ducts show low C1GALT1 expression levels.

### C1GALT1 Knockdown Suppresses CCA Cell Migration and Invasion:

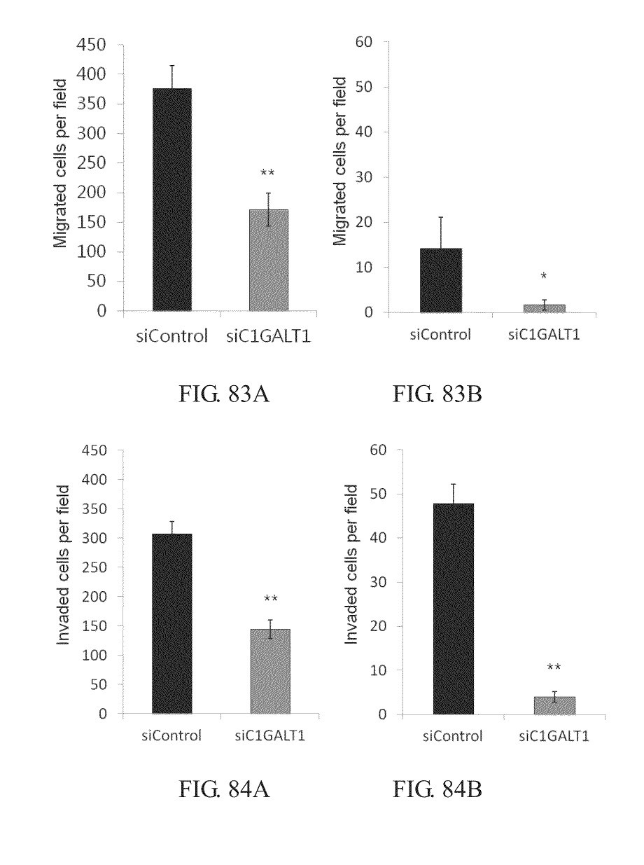

FIG. 81 is an autoradiogram illustrating C1GALT1 expression in three CCA cell lines analyzed by Western blotting in accordance with an embodiment of the present invention. Western blot of C1GALT1 expression is in three CCA cell lines, namely, HUCCT1, SNU1079, and Huh28 cells. FIG. 82 is an autoradiogram illustrating knockdown of C1GALT1 in two CCA cell lines confirmed by Western blotting in accordance with an embodiment of the present invention. Knockdown of C1GALT1 using 20 nM non-targeting (siControl) and C1GALT1 specific (siC1GALT1) siRNA in HUCCT1 and SNU1079 cells is confirmed by Western blot. FIGS. 83A-83B are schematic diagrams illustrating the effects of C1GALT1 on the migration of CCA cells in accordance with an embodiment of the present invention. FIGS. 84A-84B are schematic diagrams illustrating the effects of C1GALT1 on the invasion of CCA cells in accordance with an embodiment of the present invention. Cells (5×10⁴) of the control and C1GALT1 knocked down HUCCT1 and SNU1079 cells are seeded for transwell migration and Matrigel™ invasion assays. Using FBS 10% as a chemoattractant, the number of migrated and invaded cells per field under microscope are counted. Knockdown of C1GALT1 significantly suppressed HUCCT1 cell migration, as shown in FIG. 83A, and invasion, as shown in FIG. 84A. Similarly C1GALT1 knockdown in SNU1079 cells also exhibit suppressed cell migration, as shown in FIG. 83B and invasion, as shown in FIG. 84B. *P<0.05 and **p<0.01.

Therefore, the present invention provides a method for treating a cell proliferative disorder in a subject, comprising a step of: administering a C1GALT1 inhibition substance to the subject for inhibiting C1GALT1 expression or activity of the subject, so as to treat the cell proliferative disorder in the subject.

In an embodiment of the present invention, the C1GALT1 inhibition substance further inhibits phosphorylation or dimerization of RTKs (receptor tyrosine kinases).

In an embodiment of the present invention, the C1GALT1 inhibition substance further alters glycosylation of RTKs (receptor tyrosine kinases).

Furthermore, the RTKs are selected from a group consisting of MET (also known as hepatocyte growth factor receptor, HGFR), and FGFRs (fibroblast growth factor receptor).

In an embodiment of the present invention, the C1GALT1 inhibition substance further alters glycosylation of MUC1 (mucin 1).

In an embodiment of the present invention, the C1GALT1 inhibition substance comprises an antisense nucleotide sequence complementary to all or a part of C1GALT1 mRNA.

Furthermore, the antisense nucleotide sequence comprises UUAGUAUACGUUCAGGUAAGGUAGG (SEQ ID NO: 1) or UUAUGUUGGCUAGAAUCUGCAUUGA(SEQ ID NO: 2).

Furthermore, a concentration of the antisense nucleotide sequence administered to the subject is ranged from 0.05 nM to 1000 nM.

In an embodiment of the present invention, the cell proliferative disorder is selected from the group consisting of hepatocellular carcinoma, colorectal cancer, breast cancer, head and neck squamous cell carcinoma, lung cancer, ovarian cancer, endometrial cancer, and cholangiocarcinoma.

In an embodiment of the present invention, the cell proliferative disorder is selected from the group consisting of cell migration, cell invasion, and tumor metastasis.

In another preferable embodiment of the present invention, the C1GALT1 inhibition substance is a small molecule substance, and the molecular weight thereof is less than 900 Daltons.

In an embodiment of the present invention, the C1GALT1 inhibition substance binds to a catalytic domain of C1GALT1.

In an embodiment of the present invention, the C1GALT1 inhibition substance leads to Tn antigen accumulation in cancer cells.

In an embodiment of the present invention, the C1GALT1 inhibition substance decreases T antigen formation in cancer cells. In an embodiment of the present invention, the C1GALT1 inhibition substance is itraconazole.

In an embodiment of the present invention, the small molecular substance administered to the subject is ranged from 1 ug/ml to 100 ug/ml.

In an embodiment of the present invention, before or after administering the C1GALT1 inhibition substance to the subject, the method for treating a cell proliferative disorder in a subject further comprises a step of: administering an anti-tumor drug to the subject for producing a synergetic effect.

The present invention also provides a method for identifying a potential compound for therapeutically treating a cell proliferative disorder, comprising steps of:

administering a to-be-tested compound to cells having the cell proliferative disorder;
examining the activity and expression of C1GALT1 in the cells; and
determining whether the to-be-tested compound is the potential compound for therapeutically treating the cell proliferative disorder, wherein when the activity or expression of C1GALT1 in the cells after being treated with the to-be-tested compound is lower than a predetermined percentage of the activity and expression of the cells before being treated with the to-be-tested compound, the to-be-tested compound is determined to be the potential compound for treating cell proliferative disorder.

In summary, the technical feature of the present invention is administering a C1GALT1 inhibition substance to the subject for inhibiting C1GALT1 expression or activity of the subject, so as to treat the cell proliferative disorder in the subject.

The present invention has been described with embodiments thereof and it is understood that various modifications, without departing from the spirit of the present invention, are in accordance with the embodiments of the present invention. Hence, the embodiments described are intended to cover the modifications within the scope and the spirit of the present invention, rather than to limit the present invention.

SEQUENCE LISTING

| | |
|---|---|
| <160> | 12 |
| <170> | PatentIn version 3.5 |
| <210> | 1 |
| <211> | 25 |
| <212> | RNA |
| <213> | Artificial Sequence |
| <220> | |
| <223> | siRNA |
| <400> | 1 |
| uuaguauacg uucagguaag guagg | 25 |
| <210> | 2 |
| <211> | 25 |
| <212> | RNA |
| <213> | Artificial Sequence |
| <220> | |
| <223> | siRNA |
| <400> | 2 |
| uuauguuggc uagaaucugc auuga | 25 |
| <210> | 3 |
| <211> | 25 |
| <212> | RNA |
| <213> | Artificial Sequence |
| <220> | |
| <223> | siRNA |
| <400> | 3 |
| uaaauguacu gcgcguggag aggaa | 25 |
| <210> | 4 |
| <211> | 1092 |
| <212> | DNA |
| <213> | Homo sapiens |
| <400> | 4 |

| | |
|---|---|
| atggcctcta aatcctggct gaatttttta accttcctct | 60 |
| gtggatcagc aataggattt | |
| cttttatgtt ctcagctatt tagtattttg ttgggagaaa | 120 |
| aggttgacac ccagcctaat | |
| gttcttcgta atgatcctca tgcaaggcat tcagatgata | 180 |
| atggacagaa tcatctagaa | |
| ggacaaatga acttcaatgc agattctagc caacataaag | 240 |
| atgagaacac agacattgct | |
| gaaaacctct atcagaaagt tagaattctt tgctgggtta | 300 |
| tgaccggccc tcaaaaccta | |
| gagaaaaagg ccaaacacgt caaagctact tgggcccagc | 360 |
| gttgtaacaa agtgttgttt | |
| atgagttcag aagaaaataa agacttccct gctgtgggac | 420 |
| tgaaaaccaa agaaggcaga | |
| gatcaactat actggaaaac aattaaagct tttcagtatg | 480 |
| ttcatgaaca ttatttagaa | |
| gatgctgatt ggtttttgaa agcagatgat gacacgtatg | 540 |
| tcatactaga caatttgagg | |
| tggcttcttt caaaatacga ccctgaagaa cccatttact | 600 |
| ttgggagaag atttaagcct | |
| tatgtaaagc agggctacat gagtggagga gcaggatatg | 660 |
| tactaagcaa agaagccttg | |
| aaaagatttg ttgatgcatt taaaacagac aagtgtacac | 720 |
| atagttcctc cattgaagac | |
| ttagcactgg ggagatgcat ggaaattatg aatgtagaag | 780 |
| caggagattc cagagatacc | |
| attggaaaag aaacttttca tccctttgtg ccagaacacc | 840 |
| atttaattaa aggttatcta | |
| cctagaacgt tttggtactg gaattacaac tattatcctc | 900 |
| ctgtagaggg tcctggttgc | |
| tgctctgatc ttgcagtttc ttttcactat gttgattcta | 960 |
| caaccatgta tgagttagaa | |
| tacctcgttt atcatcttcg tccatatggt tatttataca | 1020 |
| gatatcaacc taccttacct | |
| gaacgtatac taaaggaaat tagtcaagca aacaaaaatg | 1080 |
| aagatacaaa agtgaagtta | |
| ggaaatcctt ga | 1092 |

| | |
|---|---|
| <210> | 5 |
| <211> | 20 |
| <212> | DNA |
| <213> | Artificial Sequence |
| <220> | |
| <223> | DNA primer |
| <400> | 5 |
| tgggagaaaa ggttgacacc | 20 |
| <210> | 6 |
| <211> | 20 |
| <212> | DNA |
| <213> | Artificial Sequence |
| <220> | |
| <223> | DNA primer |
| <400> | 6 |
| ctttgacgtg tttggccttt | 20 |
| <210> | 7 |
| <211> | 20 |
| <212> | DNA |
| <213> | Artificial Sequence |
| <220> | |
| <223> | DNA primer |
| <400> | 7 |
| acagtcagcc gcatcttctt | 20 |
| <210> | 8 |
| <211> | 20 |
| <212> | DNA |
| <213> | Artificial Sequence |
| <220> | |
| <223> | DNA primer |
| <400> | 8 |
| gacaagcttc ccgttctcag | 20 |
| <210> | 9 |
| <211> | 21 |
| <212> | DNA |
| <213> | Artificial Sequence |
| <220> | |
| <223> | DNA primer |
| <400> | 9 |
| ggcctcagca cctacctacc c | 21 |
| <210> | 10 |
| <211> | 21 |
| <212> | DNA |
| <213> | Artificial Sequence |
| <220> | |
| <223> | DNA primer |
| <400> | 10 |
| tccaaggcag cctccaagtc a | 21 |
| <210> | 11 |
| <211> | 20 |
| <212> | DNA |
| <213> | Artificial Sequence |
| <220> | |
| <223> | DNA primer |
| <400> | 11 |
| gcagatcagc cacatcgccc | 20 |
| <210> | 12 |
| <211> | 20 |
| <212> | DNA |
| <213> | Artificial Sequence |
| <220> | |
| <223> | DNA primer |
| <400> | 12 |
| gcccagagtg gtgacggaga | 20 |
| <210> | 13 |
| <211> | 21 |
| <212> | DNA |
| <213> | Artificial Sequence |
| <220> | |
| <223> | DNA sequence contained in |
| | shRNA plasmid |
| <400> | 13 |
| cccagcctaa tgttcttcat a | 21 |

What is claimed is:

1. A method for treating a cell proliferative disorder in a subject, comprising a step of:
administering a C1GALT1 inhibition substance to the subject for inhibiting C1GALT1 expression or activity in the subject, so as to treat the cell proliferative disorder in the subject.

2. The method as claimed in claim 1, wherein the C1GALT1 inhibition substance further inhibits phosphorylation or dimerization of receptor tyrosine kinases (RTKs).

3. The method as claimed in claim 2, wherein the RTKs are selected from a group consisting of mesenchymal epithelial transition factor (MET) and fibroblast growth factor receptors (FGFRs).

4. The method as claimed in claim 1, wherein the C1GALT1 inhibition substance further alters glycosylation of RTKs.

5. The method as claimed in claim 4, wherein the RTKs are mesenchymal epithelial transition factor (MET) or fibroblast growth factor receptors (FGFRs).

6. The method as claimed in claim 1, wherein the C1GALT1 inhibition substance further alters glycosylation of mucin 1 (MUC1).

7. The method as claimed in claim 1, wherein the C1GALT1 inhibition substance comprises an antisense nucleotide sequence complementary to all or a part of C1GALT1 mRNA.

8. The method as claimed in claim 2, wherein the antisense nucleotide sequence comprises UUAGUAUACGUUCAGGUAAGGUAGG (SEQ ID NO: 1) or UUAUGUUGGCUAGAAUCUGCAUUGA (SEQ ID NO: 2).

9. The method as claimed in claim 2, wherein a concentration of the antisense nucleotide sequence administered to the subject is ranged from 0.05 nM to 1000 nM.

10. The method as claimed in claim 1, wherein the cell proliferative disorder is selected from the group consisting of hepatocellular carcinoma, colorectal cancer, breast cancer, head and neck squamous cell carcinoma, lung cancer, ovarian cancer, endometrial cancer, and cholangiocarcinoma.

11. The method as claimed in claim 1, wherein the cell proliferative disorder is selected from the group consisting of cell migration, cell invasion, and tumor metastasis.

12. The method as claimed in claim 1, wherein the C1GALT1 inhibition substance is a small molecular substance, and the molecular weight thereof is less than 900 Daltons.

13. The method as claimed in claim 1, wherein the C1GALT1 inhibition substance binds to a catalytic domain of C1GALT1.

14. The method as claimed in claim 1, wherein the C1GALT1 inhibition substance leads to Tn antigen accumulation in cancer cells.

15. The method as claimed in claim 1, wherein the C1GALT1 inhibition substance decreases T antigen formation in cancer cells.

16. The method as claimed in claim 1, wherein the C1GALT1 inhibition substance is itraconazole.

17. The method as claimed in claim 12, wherein the small molecular substance administered to the subject is ranged from 1 ug/ml to 100 ug/ml.

18. A method for identifying a potential compound for therapeutically treating a cell proliferative disorder, comprising steps of:
administering a to-be-tested compound to cells having the cell proliferative disorder;
examining the activity and expression of C1GALT1 in the cells; and
determining whether the to-be-tested compound is the potential compound for therapeutically treating the cell proliferative disorder, wherein when the activity or expression of C1GALT1 in the cells after being treated with the to-be-tested compound is lower than a predetermined percentage of the activity and expression of the cells before being treated with the to-be-tested compound, the to-be-tested compound is determined to be the potential compound for treating the cell proliferative disorder.


SEQUENCE LISTING (13 sequences)

SEQ ID NO: 1 - RNA, 25 nt, Artificial Sequence
  siRNA
uuaguauacguucagguaagguagg

SEQ ID NO: 2 - RNA, 25 nt, Artificial Sequence
  siRNA
uuauguuggcuagaaucugcauuga

SEQ ID NO: 3 - RNA, 25 nt, Artificial Sequence
  siRNA
uaaauguacugcgcguggagaggaa

SEQ ID NO: 4 - DNA, 1092 bp, Homo sapiens
atggcctctaaatcctggctgaattttttaaccttcctctgtggatcagcaataggatttcttttatgttctcagctatttagtattttgttgggagaaaaggttgacacccagcctaatgttcttcgtaatgatcctcatgcaaggcattcagatgataatggacagaatcatctagaaggacaaatgaacttcaatgcagattctagccaacataaagatgagaacacagacattgctgaaaacctctatcagaaagttagaattctttgctgggttatgaccggccctcaaaacctagagaaaaaggccaaacacgtcaaagctacttgggcccagcgttgtaacaaagtgttgtttatgagttcagaagaaaataaagacttccctgctgtgggactgaaaaccaaagaaggcagagatcaactatactggaaaacaattaaagcttttcagtatgttcatgaacattatttagaagatgctgattggtttttgaaagcagatgatgacacgtatgtcatactagacaatttgaggtggcttctttcaaaatacgaccctgaagaacccatttactttgggagaagatttaagccttatgtaaagcagggctacatgagtggaggagcaggatatgtactaagcaaagaagccttgaaaagatttgttgatgcatttaaaacagacaagtgtacacatagttcctccattgaagacttagcactggggagatgcatggaaattatgaatgtagaagcaggagattccagagataccattggaaaagaaacttttcatccctttgtgccagaacaccatttaattaaaggttatctacctagaacgttttggtactggaattacaactattatcctcctgtagagggtcctggttgctgctctgatcttgcagtttcttttcactatgttgattctacaaccatgtatgagttagaatacctcgtttatcatcttcgtccatatggttatttatacagatatcaacctaccttacctgaacgtatactaaaggaaattagtcaagcaaacaaaaatgaagatacaaaagtgaagttaggaaatccttga

SEQ ID NO: 5 - DNA, 20 bp, Artificial Sequence
  DNA primer
tgggagaaaaggttgacacc

SEQ ID NO: 6 - DNA, 20 bp, Artificial Sequence
  DNA primer
ctttgacgtgtttggccttt

SEQ ID NO: 7 - DNA, 20 bp, Artificial Sequence
  DNA primer
acagtcagccgcatcttctt

SEQ ID NO: 8 - DNA, 20 bp, Artificial Sequence
  DNA primer
gacaagcttcccgttctcag

SEQ ID NO: 9 - DNA, 21 bp, Artificial Sequence
  DNA primer
ggcctcagcacctacctaccc

SEQ ID NO: 10 - DNA, 21 bp, Artificial Sequence
  DNA primer
tccaaggcagcctccaagtca

SEQ ID NO: 11 - DNA, 20 bp, Artificial Sequence
  DNA primer
gcagatcagccacatcgccc

SEQ ID NO: 12 - DNA, 20 bp, Artificial Sequence
  DNA primer
gcccagagtggtgacggaga

SEQ ID NO: 13 - DNA, 21 bp, Artificial Sequence
  DNA sequence contained in shRNA plasmid
cccagcctaatgttcttcata